(19) United States
(12) Patent Application Publication
(10) Pub. No.: US 2025/0302711 A1
(43) Pub. Date: Oct. 2, 2025

(54) METHOD FOR CLEANSING HUMAN KERATIN MATERIALS

(71) Applicant: L'OREAL, Paris (FR)

(72) Inventors: Laure DESFORGES, Chevilly Larue (FR); Dominique BORDEAUX, Chevilly Larue (FR); Veronique Bumier-Yalaoui, Chevilly Larue (FR)

(21) Appl. No.: 18/864,208

(22) PCT Filed: May 17, 2023
(86) PCT No.: PCT/EP2023/063309

§ 371 Date: Nov. 8, 2024

(30) Foreign Application Priority Data
May 18, 2022 (FR) FR2204748

(51) Int. Cl. A61K 8/36; A61Q 1/14; A61Q 5/02

(52) U.S. Cl. CPC A61K 8/361; A61Q 1/14; A61Q 5/02; A61K 2800/82

(57) ABSTRACT

Method for cleansing human keratin materials, including subjecting a surface of said keratin materials to a cosmetic composition containing at least one surfactant, and to acoustic waves emitted by at least one ultrasonic transducer excited by a pulsed or non-pulsed electrical signal, having at least one frequency component of between 20 kHz and 100 kHz, with a duty cycle Ton/Toff, when the signal is pulsed, of between 20 and 100%, with an acoustic intensity Isata on said surface of at least 0.1 W/cm2 and with a pulse duration Ton, when the signal is pulsed, of between 0.01 and 1 s, so as to generate, within the cosmetic composition, bubbles in the vicinity of the surface of said materials to be cleansed and to cause them to burst.

### TECHNICAL FIELD

The present invention relates to the cleaning of human keratin materials, and more particularly, but not exclusively, to the treatment of the skin, the scalp and/or the hair, in particular hair that has been dyed.

### PRIOR ART

Application JP 2007-311756 describes an ultrasonic cleaning device for cleaning a silicon wafer, a mask substrate, etc. The device comprises an ultrasonic transducer mounted in a nozzle that is passed through by a cleaning liquid, and a means for introducing a gas in order to generate bubbles in the cleaning liquid.

Application US 2012/0227761 describes a device for cleaning a surface, comprising, on the one hand, a chamber that is fed with a liquid and communicates with an outlet duct opening out on the surface to be cleaned, an ultrasonic transducer transmitting acoustic energy to the liquid contained in the chamber and the outlet duct, and, on the other hand, a bubble generator for generating bubbles of a gas in the outlet duct. The bubble generator may be an electrochemical generator, the liquid containing for example a salt, such as potassium chloride, in order to make the liquid electrically conductive. A surfactant may be added in order to prevent the bubbles coalescing during their journey in the outlet duct to the surface to be treated, and ensure that they have the required size when they reach the surface to be treated.

The application of the device to cleaning the skin, in particular under the nails of a surgeon, and also the hands, is envisaged.

That application mentions the possible addition of surfactant in order to influence the size of the bubbles that are produced. No cosmetic application for treating the facial skin or the hair is disclosed.

Application ES 2 708 149 discloses a device for cleaning a surface comprising a sonotrode generating ultrasound close to a cleaning solution present on the surface, cavitation making it possible to remove dirt from the surface.

U.S. Pat. No. 8,486,199 describes a device for treating a surface of a semiconductor wafer comprising a resonator for supplying ultrasonic energy to a treatment fluid containing gas bubbles.

Document EP 1 645 342 describes a method for cleaning items of equipment, consisting in subjecting a liquid containing bubbles to an acoustic field.

Patent KR101152920 describes a device for cleaning the skin, comprising a vacuum pump and intended to be used close to a liquid containing microbubbles on the skin.

Applications US 2017 0080257, KR 2020 0102956, US 2009/318853, JP 2016 214424 and WO 2020/029429 disclose methods for the cosmetic treatment of the skin, in particular for cleaning it, using an ultrasonic device that generates microbubbles in a cosmetic composition that is applied to the skin to be cleaned.

Similar devices are disclosed in applications US 2011/21328, US 2010/010420 and EP 3 634 643, CN206995178 and EP 2 470 310.

Application EP 3 542 740 discloses a medical device for cleaning wounds, comprising a removable cleaning nozzle and an ultrasonic transducer.

Some devices involve a significant amount of water and new compositions each time they are used.

The environmentally friendly design of products promoting the sustainable use of resources has become an essential key factor for minimizing the impact of products on the environment. Producers have become accountable and are incited, by changes in consumer habits, to design their formulas and packaging in an environmentally friendly manner, while still taking care to optimize industrial processes and to manage production waste. A virtuous circle is thus constructed. Likewise, helping consumers to be less wasteful is contributing to this global movement of everyone becoming accountable.

There is therefore a need for products with a reduced environmental footprint, able to address the increasingly real expectations of consumers.

Moreover, when it is desired to apply a dyeing treatment to hair that has already been dyed, it may be desirable to first bleach said hair; such a bleaching treatment however has to be carried out, ideally, without damaging the hair fiber.

Now, bleaching is carried out at present using compounds having a chemical action on the hair, such as oxidizers, which may have an impact on the environment, and it would be desirable to avoid using these or to reduce the amounts thereof. In addition, these compounds may have a relatively aggressive action on the hair fiber.

Indeed, treatments involving dyeing or bleaching the hair are generally carried out using an oxidizing agent, such as hydrogen peroxide, a potassium salt, sodium salt, ammonium salt, perborate salt or percarbonate salt, persulfate or percarbamide, in an alkaline solution. The oxidizing agents used in these hair treatments generally cause breakage of the disulfide bonds linking the keratin chains. Repeated treatments with oxidizing agents therefore often result in the hair being made fragile and brittle, and making it lose its shine.

Conditioning agents, such as silicones, cationic surfactants or cationic polymers, have been proposed for application to the hair following rinsing of the composition containing the oxidizing agents.

These conditioning agents make it possible to attenuate damage caused to the hair by creating a protective film, thereby improving the touch of the hair, but do not prevent premature breakage of the hair able to be caused by successive oxidizing treatments. Moreover, these conditioning agents have a significant impact on the environment.

Numerous solutions have been proposed to attempt to address this problem.

U.S. Pat. No. 5,100,436 thus discloses hair dye compositions comprising metal-chelating agent complexes making it possible to reduce the time for which the hair is exposed to oxidizing compositions, thus reducing the damage caused by the oxidizing agent.

U.S. Pat. No. 6,013,250 discloses a composition for treating the hair against chemical damage and photo-damage. U.S. Pat. No. 4,138,478 discloses the use of a specific compound to reduce damage caused to the hair during bleaching thereof or dyeing thereof. U.S. Pat. Nos. 3,202,579 and 3,542,918 disclose other protective compounds. U.S. Pat. No. 5,635,167 discloses a method for eliminating exogenous metal ions attached to the hair, comprising a step of putting the hair in contact with a mixture of chelating agents. WO97/24106 discloses hair dye compositions comprising a whitening additive for reducing damage caused to the hair, with a low pH.

All of these solutions are based on the use of specific chemical compounds, and do not address the need to improve the environmental footprint of treatments for dyeing and/or bleaching the hair or cleaning the hair in a completely satisfactory manner. Moreover, they do not completely prevent damage caused to the hair by the action of bleaching agents such as oxidizers.

### DISCLOSURE OF THE INVENTION

The invention aims to propose a method for cleaning human keratin materials, in particular the facial skin, the scalp or the hair, that makes it possible to effectively clean said materials, and is compatible with a responsible sustainable development approach by virtue of a reduced carbon footprint.

The invention also aims to propose a device and a method for treating the human hair that make it possible to reduce, as far as possible, the use of chemical products for bleaching the hair, to limit the consumption of water and/or active agents and to dispense with substances harmful to the environment, to reduce the risks linked to repeatedly exposing the hair and the scalp to aggressive substances and treatments, and to facilitate treatments for bleaching and dyeing the hair.

### SUMMARY OF THE INVENTION

One subject of the invention, according to a first of its aspects, is thus a method for treating human keratin materials, comprising the step of subjecting a surface of said keratin materials to a cosmetic composition containing at least one surfactant, and to acoustic waves emitted by at least one ultrasonic transducer excited by a pulsed or non-pulsed electrical signal, having at least one frequency component of between 20 kHz and 100 kHz, with a duty cycle Ton/Toff, when the signal is pulsed, of between preferably 20 and 100%, with a pulse duration Ton, when the signal is pulsed, of between preferably 0.01 and 1 s, and with an acoustic intensity Isata on said surface of preferably at least 0.1 W/cm².

It is thereby possible, within the cosmetic composition, to generate bubbles in the vicinity of the surface of said materials to be cleaned and to cause said bubbles to burst.

This method is preferably cosmetic, for non-therapeutic purposes.

A “cosmetic composition” denotes a composition containing at least one cosmetic active agent, as defined in the 76/768/EEC Cosmetics Directive. It may in particular be an active agent contributing to the cleaning and/or the bleaching of the hair, as described below. According to the invention, mineral water or tap water does not constitute a cosmetic composition.

“Human keratin materials” denotes external keratin materials such as the skin and the appendages, in particular the hair and the nails, and internal keratin materials, such as the gums or other mucous membranes. The treatment may in particular be carried out on the surface layers of the skin. The area of skin that is treated according to the invention may be the facial skin, the skin on the torso, the back, the arms, the legs, the hands and/or the feet, the scalp. The method according to the invention is very particularly suitable for removing make-up and/or cleaning the facial skin, in particular the forehead, the cheeks, the chin, the neck, the nose, the scalp.

The method according to the invention may also be suitable for caring for the skin or the hair, or for cleaning and/or preparing the skin, the hair or the scalp, in particular for preparing the hair for a dyeing or bleaching treatment.

“At least one frequency component” is understood to mean frequency in its conventional sense for a periodic signal the spectrum of which contains a single line, for example a sinusoidal line, and, for a periodic signal the spectrum of which contains multiple lines, the frequency of at least one line, in particular the one with the highest amplitude.

The acoustic intensity ISATA may be defined as the average acoustic power over the surface area subjected to the acoustic waves, divided by said surface area:

$${I}_{SATA} = {P}_{moy} / S ,$$

the average acoustic power being obtained from the activation voltage of the transducer, and from the duty cycle when the electrical signal is pulsed.

The acoustic intensity ISATA is preferably between 0.1 and 10 W/cm², better still between 1 and 5 W/cm², even better still between 2.5 and 5 W/cm², even better still between 3 and 4 W/cm².

The method according to the invention, by virtue of the abovementioned parameter ranges for the signal, and in particular a minimum acoustic intensity ISATA, makes it possible to eliminate or to effectively treat both exogenous impurities and endogenous impurities or defects.

Among undesirable soiling possibly present on keratin materials (on the surface and/or anchored more deeply in the pores of the skin), it is possible to distinguish between exogenous impurities such as make-up, environmental pollution, dust, microorganisms, etc., and endogenous impurities or defects such as excess sebum, sweat, dead cells, dead skin, dandruff, blackheads, small scars and/or acne, pigmented spots, etc.

The acoustic waves obtained using the abovementioned parameter ranges for the signal make it possible to bring about complete cavitation of bubbles within the composition in order to generate a mechanical effect on the keratin materials to be cleaned.

“Complete cavitation” is understood here to mean both the generation and the bursting of the bubbles at the origin of a mechanical shock on the nearby surfaces. The cosmetic composition may or may not have bubbles already present inside it before the acoustic waves are applied.

The bubbles that are generated and/or present may also contribute to the release of chemical species that contribute to the cleaning of the keratin materials, such as free radicals.

### Surfactant

The composition preferably has a total concentration of surfactant(s) of between 0.01% and 20% by weight relative to the total mass of the composition, better still between 0.01% and 5%, even better still 0.1% and 1.5%; the composition may thus also have a cleaning action in the absence of the bursting of the bubbles by the acoustic waves.

The surfactants may contribute to the formation of the bubbles and/or to the stabilization thereof, being chosen for example from among foaming surfactants such as anionic polyoxyalkylenated alkyl(amido) ether carboxylic acid surfactants, anionic surfactants other than the abovementioned polyoxyalkylenated alkyl(amido) ether carboxylic acids, non-ionic surfactants, amphoteric and zwitterionic surfactants and mixtures thereof and/or are chosen from the compounds conventionally present within make-up removal compositions such as alkyl polysaccharides, fatty alcohol polyethylene glycols, oils and mixtures thereof. Any surfactant able to generate micelles in the medium may be used.

### Acoustic Waves

The acoustic waves may be generated by a single transducer or, as a variant, by at least two transducers.

When the acoustic waves are emitted by multiple transducers, these are for example all directed at the area to be treated, having for example longitudinal axes convergent thereon. It is possible to use, where applicable, two transducers arranged facing one another in order to treat a strand of hair inserted between them. This may make it possible to simultaneously treat two opposing sides of the strand. It is also possible to have transducers arranged side by side to treat a larger area, with the areas treated by each of them overlapping or not overlapping.

Each transducer comprises one or more electroactive elements for converting an electric current into mechanical vibrations, these one or more electroactive elements being for example based on piezoelectric materials, as described below.

These one or more electroactive elements may be coupled to a sonotrode that may be set into mechanical resonance by the one or more electroactive elements and serves to define an emission surface for emitting acoustic waves toward the surface or the target volume.

The acoustic waves may be generated continuously as soon as the treatment device is put into operation or, as a variant, be generated only when certain operating conditions are met, such as for example the presence of the composition in contact with the sonotrode and/or the presence of the device in contact with the area to be treated and/or if the device detects that the area comprises soiling.

As a variant, the acoustic waves may be generated only when the amount of composition in contact with the surface to be cleaned is sufficient, or else when certain conditions for generating cavitation are detected, such as contact between the emission surface of the sonotrode and the surface to be treated, a minimum composition height, a composition temperature or transducer temperature within a given range, or the like.

The acoustic waves may be generated by a pulsed or continuous electrical signal, preferably a pulsed electrical signal.

They may be generated by a sinusoidal electrical signal or by a signal with a more complex waveform, for example with frequency modulation or with amplitude modulation. The acoustic waves are preferably emitted at a single frequency, thereby making it possible to focus them more precisely within a given area but, as a variant, may be emitted at multiple different frequencies.

Said frequency is preferably between 30 and 100 kHz, in particular between 30 and 45 kHz. Such a frequency allows both satisfactory generation and bursting of the bubbles in the composition, and bursting of just any bubbles already present in the medium in the composition. The acoustic waves are for example generated by a transducer with a nominal frequency of around 34 kHz.

The nominal frequency may vary around 34 kHz, for example 34 kHz+/−5 kHz.

The electrical signal exciting the one or more transducers may be pulsed with a pulse duration of preferably between 0.01 s and 0.1 s, better still between 0.02 and 0.08 s.

The duty cycle, when the signal is pulsed, is preferably between 20% and 100%, better still between 50% and 70%, for example roughly equal to 50%+/−10%, 58.3%+/−10% or 66.7%+/−10%.

Some variations with respect to a nominal value may result where applicable from the temperature rise of the transducer, through for example the excitation of the ceramics forming a piezoelectric electroactive element. These variations may also stem from the temperature rise of the liquid in which the cavitation is generated.

The method may comprise a step of detecting the presence of the cosmetic composition in contact with the sonotrode, and making the operation of the transducer conditional on this detection.

This thus avoids emitting acoustic waves in the absence of composition or when the device is not in a situation of treating keratin materials.

Preferably, the acoustic waves are emitted without the emission surface of the sonotrode coming into contact with the area to be treated. The distance between the transducer and the surface to be treated is for example between 1 and 30 mm, more particularly between 3 and 5 mm, thereby making it possible to limit the acoustic energy emitted into the keratin materials, and to promote the cavitation of the bubbles in the composition.

“The distance between the transducer and the surface to be treated” should be understood as being the distance between the emission surface of the sonotrode and the surface to be treated.

It is possible to channel a composition continuously in contact with the sonotrode, with continuous operation of the one or more transducers, or as a variant pulsed operation of the one or more transducers. The composition may also be supplied in a pulsed manner. There is advantageously a discontinuous supply to an area subjected to the acoustic waves, with for example a supply period to the area while the acoustic waves are not being generated, and then a period in which the composition present in the area is subjected to the acoustic waves without being replenished during this exposure. This may make it possible to avoid bubbles present in the composition, before they enter said area, being repelled by the acoustic waves present in said area.

It is thus possible to slave the supply of composition to the treatment area to the operation of the one or more transducers for the production of the acoustic waves or vice versa, and have pulsed operation of the one or more transducers and/or of the supply of composition. In other words, it is possible to supply composition with a time delay with respect to the period of emission of the acoustic waves into the area where the composition is supplied.

The propagation of the acoustic waves is facilitated by the presence of liquid in the composition. The gas/liquid weight ratio within the composition may thus benefit from not being excessively high, and the composition may advantageously contain a thickener, as described below, or any other compound for increasing the temporal stability of the bubbles.

Preferably, an activation voltage is delivered to the transducer in order to obtain a peak acoustic intensity I greater than or equal to 5 W/cm². The peak intensity I is preferably between 5 and 10 W/cm², better still between 6 and 7.5 W/cm².

The surface to be treated is preferably subjected to the acoustic waves for a duration of between 0.2 s and 10 s, better still between 2 and 5 s, for example 4 s+/−0.4 s. Such an “effective” application duration of the acoustic waves allows effective cleaning while still limiting any sensation of discomfort for the user. The duration may correspond to the time measured between the start of excitation of the transducer for treating a given surface and the end of excitation thereof in the treatment of said surface. This time may be applied in one go or in multiple additional goes.

The surface to be treated is for example subjected to the acoustic waves for a duration equal to 4 s+/−0.4 s, the peak acoustic intensity is equal to 6.2+/−0.6 W/cm², and:
the composition has a total concentration of surfactant(s) of between 0.8 and 1.5%, the pulse duration Ton is between 0.020 and 0.080 s and the duty cycle is between 50 and 67%, or
the composition has a total concentration of surfactant(s) equal to 0.5%+/−0.1%, and
   the pulse duration Ton is between 0.025 and 0.080 s and the duty cycle is between 50 and 67%, or
   the pulse duration Ton is between 0.020 and 0.080 s and the duty cycle is between 52 and 65%.

As a variant, the surface is for example subjected to the acoustic waves for a duration equal to 4 s+/−0.4 s, the peak acoustic intensity is equal to 6.8 W/cm²+/−0.7 W/cm², and:
the composition has a total concentration of surfactant(s) equal to 0.5%+/−0.1%, and
   the pulse duration Ton is between 0.027 and 0.080 s and the duty cycle is between 50 and 67%, or
   the pulse duration Ton is between 0.020 and 0.080 s and the duty cycle is between 52 and 67%,
or
the composition has a total concentration of surfactant(s) equal to 0.8%+/−0.1%, and
   the pulse duration Ton is between 0.030 and 0.080 s and the duty cycle is between 50 and 67%, or
   the pulse duration Ton is between 0.020 and 0.080 s and the duty cycle is between 52 and 67%,
or
the composition has a total concentration of surfactant(s) equal to 1.0%+/−0.1%, and
   the pulse duration Ton is between 0.035 and 0.080 s and the duty cycle is between 50 and 67%, or
   the pulse duration Ton is between 0.020 and 0.080 s and the duty cycle is between 52.5 and 67%,
or
the composition has a total concentration of surfactant(s) equal to 1.2%+/−0.1%, and
   the pulse duration Ton is between 0.035 and 0.080 s and the duty cycle is between 51.5 and 67%, or
   the pulse duration Ton is between 0.020 and 0.080 s and the duty cycle is between 54 and 67%,
or
the composition has a total concentration of surfactant(s) equal to 1.5%+/−0.1%, and
   the pulse duration Ton is between 0.038 and 0.080 s and the duty cycle is between 54 and 67%, or
   the pulse duration Ton is between 0.020 and 0.080 s and the duty cycle is between 57.5 and 67%.

As a variant, the surface to be treated is for example subjected to the acoustic waves for a duration equal to 4 s+/−0.4 s, the peak acoustic intensity is equal to 7.2 W/cm²+/−0.7 W/cm², and:
the composition has a total concentration of surfactant(s) equal to 0.5%+/−0.1%, the pulse duration Ton is between 0.020 and 0.080 s and the duty cycle is between 50 and 67%,
or
the composition has a total concentration of surfactant(s) equal to 0.8%+/−0.1%, and
   the pulse duration Ton is between 0.030 and 0.070 s and the duty cycle is between 51 and 67%, or
   the pulse duration Ton is between 0.020 and 0.080 s and the duty cycle is between 52.5 and 67%,
or
the composition has a total concentration of surfactant(s) equal to 1.0%+/−0.1%, and
   the pulse duration Ton is between 0.030 and 0.070 s and the duty cycle is between 52.5 and 67%, or
   the pulse duration Ton is between 0.020 and 0.080 s and the duty cycle is between 55 and 67%,
or
the composition has a total concentration of surfactant(s) equal to 1.2%+/−0.1%, and
   the pulse duration Ton is between 0.025 and 0.070 s and the duty cycle is between 56 and 67%, or
   the pulse duration Ton is between 0.020 and 0.080 s and the duty cycle is between 59 and 67%,
or
the composition has a total concentration of surfactant(s) equal to 1.5%+/−0.1%, the pulse duration Ton is between 0.028 and 0.045 s and the duty cycle is between 59 and 63%.

As a variant, the surface to be treated is for example subjected to the acoustic waves for a duration equal to 3 s+/−0.3 s, the peak acoustic intensity is equal to 6.2 W/cm²+/−0.6 W/cm², and:
the composition has a total concentration of surfactant(s) equal to 0.5%+/−0.1%, and
   the pulse duration Ton is between 0.070 and 0.080 s and the duty cycle is between 54 and 60.5%, or
   the pulse duration Ton is between 0.055 and 0.078 s and the duty cycle is between 56 and 62%,
or
the composition has a total concentration of surfactant(s) equal to 0.8%+/−0.1%, and
   the pulse duration Ton is between 0.040 and 0.080 s and the duty cycle is between 51 and 67%, or
   the pulse duration Ton is between 0.020 and 0.080 s and the duty cycle is between 56 and 67%,
or
the composition has a total concentration of surfactant(s) equal to 1.0%+/−0.1%, and
   the pulse duration Ton is between 0.035 and 0.080 s and the duty cycle is between 51.5 and 67%, or
   the pulse duration Ton is between 0.020 and 0.080 s and the duty cycle is between 54 and 67%,
or
the composition has a total concentration of surfactant(s) equal to 1.2%+/−0.1%, and
   the pulse duration Ton is between 0.040 and 0.080 s and the duty cycle is between 51.5 and 67%, or
   the pulse duration Ton is between 0.020 and 0.080 s and the duty cycle is between 54 and 67%,
or
the composition has a total concentration of surfactant(s) equal to 1.5%+/−0.1%, and
   the pulse duration Ton is between 0.035 and 0.080 s and the duty cycle is between 55 and 67%, or
   the pulse duration Ton is between 0.020 and 0.080 s and the duty cycle is between 57 and 67%.

As a variant, the surface to be treated is for example subjected to the acoustic waves for a duration equal to 3 s+/−0.3 s, the peak acoustic intensity is equal to 6.8 W/cm²+/−0.7 W/cm², and:
the composition has a total concentration of surfactant(s) equal to 0.5%+/−0.1%, and
   the pulse duration Ton is between 0.020 and 0.065 s and the duty cycle is between 56 and 67%, or
   the pulse duration Ton is between 0.035 and 0.075 s and the duty cycle is between 53.5 and 64%,
or
the composition has a total concentration of surfactant(s) equal to 0.8%+/−0.1%, and
   the pulse duration Ton is between 0.030 and 0.072 s and the duty cycle is between 53 and 67%, or
   the pulse duration Ton is between 0.020 and 0.080 s and the duty cycle is between 55 and 64.5%,
or
the composition has a total concentration of surfactant(s) equal to 1.0%+/−0.1%, and
   the pulse duration Ton is between 0.032 and 0.070 s and the duty cycle is between 54 and 67%, or
   the pulse duration Ton is between 0.020 and 0.080 s and the duty cycle is between 56 and 64.5%,
or
the composition has a total concentration of surfactant(s) equal to 1.2%+/−0.1%, and
   the pulse duration Ton is between 0.032 and 0.070 s and the duty cycle is between 55.5 and 67%, or
   the pulse duration Ton is between 0.020 and 0.080 s and the duty cycle is between 58 and 64%,
or
the composition has a total concentration of surfactant(s) equal to 1.5%+/−0.1%, and
   the pulse duration Ton is between 0.025 and 0.063 s and the duty cycle is between 61.5 and 67%, or
   the pulse duration Ton is between 0.030 and 0.058 s and the duty cycle is between 60 and 67%.

As a variant, the surface to be treated is for example subjected to the acoustic waves for a duration equal to 3 s+/−0.3 s, the peak acoustic intensity is equal to 7.2 W/cm²+/−0.7 W/cm², and:
the composition has a total concentration of surfactant(s) equal to 0.5%+/−0.1%, and
   the pulse duration Ton is between 0.020 and 0.080 s and the duty cycle is between 57 and 65%, or
   the pulse duration Ton is between 0.030 and 0.065 s and the duty cycle is between 55 and 67%,
or
the composition has a total concentration of surfactant(s) equal to 0.8%+/−0.1%, and
   the pulse duration Ton is between 0.020 and 0.080 s and the duty cycle is between 58 and 67%, or
   the pulse duration Ton is between 0.020 and 0.068 s and the duty cycle is between 55 and 67%,
or
the composition has a total concentration of surfactant(s) equal to 1.0%+/−0.1%, and
   the pulse duration Ton is between 0.020 and 0.0725 s and the duty cycle is between 58.5 and 67%, or
   the pulse duration Ton is between 0.020 and 0.0575 s and the duty cycle is between 56 and 67%,
or
the composition has a total concentration of surfactant(s) equal to 1.2%+/−0.1%, and
   the pulse duration Ton is between 0.020 and 0.060 s and the duty cycle is between 61 and 67%, or
   the pulse duration Ton is between 0.020 and 0.046 s and the duty cycle is between 58.5 and 67%.

### Gas Bubbles

Bubbles may be generated during pressure drops in the composition and the lowering of the vapor pressure that generates the release of gases. Preferably, the bubbles are generated by the acoustic waves but, as a variant, they are additionally generated by a specific generator.

The gas bubbles may be bubbles of air, CO₂, oxygen, hydrogen, nitrogen, among other possibilities, and also a mixture of these gases.

All of the bubbles may be bubbles of the same gas or, as a variant, the composition may contain bubbles of a first gas and bubbles of a second gas, different from the first.

The gas may stem from decomposition of the composition, for example through fermentation, or be extracted therefrom, or as a variant introduced into the composition. The diameter of the bubbles may range from 50 nm to 700 μm, better still from 500 nm to 50 μm. The size here denotes the average size D₅₀ by number at half the population.

The non-dimensional factor is equal to the ratio d/Rₘₐₓ, where d is the distance from the geometric center of the bubble to the surface to be cleaned when the expansion of the bubble is at a maximum, and Rₘₐₓ is the maximum expansion diameter of the bubble, is preferably less than 3.5, better still less than 1.1, so as to have maximum effectiveness, as described in the publication “Mechanisms of single bubble cleaning”, F. Reuter, Ultrasonics Sonochemistry 29 (2016) 550-562.

The density of the medium formed by the cosmetic composition with the gas bubbles, exposed to the acoustic waves, may be between 0.1 and 1 g/cm³, better still between 0.5 g and 1 g/cm³ (at 20° C. and at atmospheric pressure).

A small bubble size may facilitate penetration thereof into reliefs and/or follicles in the skin, such as hair follicles, cracks, wrinkles, scars, crevices or folds, and thus exert an effective cleaning action within them. It may thus be beneficial for the bubbles to have sizes less than or equal to 300 microns, better still 200 microns, for example a size of 100 microns or less. The bubbles may then penetrate into the follicles before being activated by the acoustic waves.

### Additional Generation of Bubbles

Preferably, the bubbles are generated only by the acoustic waves in the cavitation phenomenon, obtained in particular through a minimum level of Isata and through the other abovementioned parameters, as described above.

As a variant, additional bubbles may be generated within the composition. These additional bubbles may be generated by any appropriate means, for example by a mechanical, physical, chemical or electrochemical means. The bubbles may in particular be generated by a depression in the liquid that makes it possible to lower the vapor pressure and to generate the formation of gas in the form of bubbles.

The additional bubbles may be generated prior to, at the same time as or cyclically with respect to the emission of the acoustic waves.

The following techniques, among others, constitute exemplary techniques able to be implemented to generate the bubbles in the invention:
decompression of liquids using nozzles for example, the decompression possibly being preceded by pressurization,
creation of a turbulent flow, in particular using rotating blades, a turbine, an ejector, a Venturi, a flow of swirling gas or liquid, a static or rotating porous body, in particular a rotating disk, a generator as described in the publication Performance of a new micro-bubble generator with a spherical body in a flowing water tube by M. Sadatomi Experimental Thermal and Fluid Science 29 (2005) 615-623, a Venturi tube and a vortex, a Venturi as implemented by MEC Co. (Iona Shower), or shear forces,
creation of a laminar flow through small openings, with focusing or co-focusing of the flow, a porous membrane, using a porous-membrane fluidic oscillator as described for example in the article Fluidic oscillator-mediated microbubble generation to provide cost effective mass transfer and mixing efficiency to the wastewater treatment plants Environ Res. 2015 February; 137:32-9, an emulsification microchannel as described in the article Monodispersed microbubble formation using microchannel technique; AIChE Journal, 50 (2004), pp. 3227-3233, these techniques for creating bubbles through a laminar flow through small openings being preferred to the previous ones due to the stability of the bubbles that are produced.
supplying
light energy, for example by irradiating nanoporous gold-coated microfluidic channels, as described in Progress in Biomedical Optics and Imaging Proceedings of SPIE Volume 9705, 2016 Article number 97050D, Photothermal generation of microbubbles on plasmonic nanostructures inside microfluidic channels (Conference paper), by laser irradiation, for example as described in An Experimental study on microbubble generation by laser induced breakdown in water; The review of Laser Engineering (Suppl.) (2008), pp. 1273-1275 (2), by laser irradiation on carbon nanotubes, as described in the article Producing single microbubbles with control size using microfiber Advance in Bioscience and Biotechnology, 2 (2011), pp. 385-390, by a tapered optical fiber, by a plasmonic absorber in the near infrared, as described in the publication Size-controllable micro-bubble generation using a nanoimprinted plasmonic nanopillar array absorber in the near-infrared region; Applied Physics Letters. 108. 2016,
acoustic energy, for example by ultrasound as described in the article Ultrasonics Sonochemistry Volume 29, Mar. 1, 2016, pp. 604-611, Influence of sonication conditions on the efficiency of ultrasonic cleaning with flowing micrometer-sized air bubbles, sonic activation of microbubbles, ultrasound in the presence of nucleation sites, as described in application WO2016/055883,
electrical energy, for example by an electrodynamic coaxial atomization method as described in the publication Preparation of microbubble suspensions by co-axial electrohydrodynamic atomization, Medical Engineering and Physics, 29 (2007), pp749-754, by electrolysis, by electro-flotation, by an electrolytic microgenerator as described in the publication Micro-fabricated electrolytic micro-bubblers, International Journal of Multiphase Flow, 31 (2005), pp. 706-722, by electrostatic spraying as described in the publication Microbubble generation for environmental and industrial separations, Separation and Purification Technology, 11 (1997), pp. 221-232, by electrically heating carbon nanotubes as described in the publication Microbubble generation with micro-watt power using carbon nanotubes heating elements; Proceedings of the 7th IEEE International Conference on Nanotechnology, Aug. 2-5, 2007, Hong Kong (2007) or by microplasma, as described in the article Journal of Physics D; Applied Physics Volume 47 Issue 35, Sep. 3, 2014, Article number 355203; Microbubble generation by microplasma in water.

The additional bubbles may be generated continuously, as soon as the device is put into operation. As a variant, the bubbles are generated intermittently, for example only when the composition is distributed, or periodically at a predefined frequency so as to give them time to disperse. A certain quantity of bubbles may be present in the composition before the device is activated. As a variant, the bubbles may be generated beforehand or without the device being put into operation.

The production intensity of the bubbles may be constant or variable, and where applicable adjustable by the user or automatically by the device depending on the desired result or on at least one operating parameter.

The bubbles may be generated by any one of the techniques mentioned above, in particular by injecting a pressurized gas into the composition, by way of a pump for example or a compressed gas tank, by electrolysis of the cosmetic composition, by stirring of the composition, suctioning a gas into the composition, or vaporizing a liquefied gas mixed into the composition or dissolved into the composition. The bubbles may result from the reaction of two liquids or of a liquid and at least one solid in the form for example of powder, granules, tablets, or any other form.

The flow of liquid containing the bubbles, if the bubbles are generated upstream, may range from 0.01 mL/s to 10 mL/s.

### Abrasion

The method may comprise abrasion of the keratin materials using abrasive particles and/or by part of the device in contact with the keratin materials.

This abrasion may be carried out by a device other than the one that emits the acoustic waves, by the device itself, or by a specific part mounted on the device, such as a grating, blade, scraper or any other appropriate accessory.

The part for carrying out the abrasion is for example installed in order to carry out the abrasion, and then removed at the time of the treatment performed by the bubbles and acoustic waves, for example being interchangeable with a part of the device used for the treatment performed by the acoustic waves and bubbles.

The part for carrying out the abrasion may also be present during the treatment with the bubbles and acoustic waves, as will be described below. This part may then have the form of a grating, among other possibilities.

The abrasion may be performed prior to the cosmetic composition and the bubbles being exposed to the acoustic waves, or as a variant at the same time. When the abrasion is beforehand, it may be performed by any means, in particular by mechanical or chemical action.

The abrasion of the keratin materials may be caused not only by the shockwave phenomenon that follows the exposure of the bubbles to the acoustic waves, but also at least partially by the action of abrasive particles making contact with the surface to be treated, these abrasive particles being for example present in the composition or on a surface of the device coming into contact with the external keratin materials.

The abrasive particles present in the cosmetic composition may be insoluble in the medium of the composition or, as a variant, soluble therein, and preferably then generate a gas when they dissolve, which gas will then serve to generate all or some of the bubbles subjected to the acoustic waves. The abrasive particles may be formed by a precipitation reaction linked to a chemical reaction.

The abrasive particles may be chosen from among abrasive powders of materials having a Moh's scale hardness greater than or equal to 3, for example powdered alumina, powdered silica, powdered aluminosilicates, powdered carbonates, or powdered silica-coated, alumina-coated or aluminosilicate-coated material.

They may also be powdered fruit stones, in particular apricot stones, wood cellulose, for example chopped bamboo stem, chopped coconut shell, oyster shell, seashell, sand, silicas, or a synthetic material such as polyamide, or mixed particles combining organic and inorganic compounds, and particles coated with the above compounds.

The abrasive particles may have a size of between 0.1 and 500 microns, in particular between 0.1 and 50 microns for the treatment of the hair, between 10 and 300 microns for the treatment of the scalp or the facial skin.

The solid particles used to exert an abrasive action may have a flat, spherical, elongate, polyhedral or irregular shape.

Abrasive particles may be added to the device or the liquid during the treatment.

The device may contain a chamber dedicated to the controlled storage and distribution of the abrasive particles or a compound allowing these particles to be formed through reaction or precipitation.

### Recycling

Preferably, the cosmetic composition is sent into contact with the keratin materials so as to be able to be at least partially recovered in order to be recycled.

The composition that is recovered may be filtered in order to have solid debris or its particle phase removed therefrom before being returned to the surface to be cleaned.

Preferably, the composition is recovered by suctioning or by absorption, for example using a porous medium or a suction system, in particular using a pump, as described below.

### Application of the Cosmetic Composition

Generally speaking, the composition may in itself, due to its formulation, already contribute to the removal of impurities that it is desired to remove through the action of the bubbles subjected to the acoustic waves. The action of the bubbles subjected to the acoustic waves may speed up or improve this process. The association of the wave generated by the bursting of the bubbles in combination with the action of the combination may thus, by synergy, have an effect greater than that of just the waves or than that of just the composition.

The cosmetic composition may be applied first of all, with bubbles already present within it or not, and then exposed, second of all, after it has been applied to the keratin materials, to the acoustic waves in order to generate new bubbles, and bring about shockwaves following the bursting of all of the bubbles.

For example, the user first applies a cosmetic composition, for example in the form of foam, to the area to be cleaned, for example by spraying it onto said area, and then brings a treatment device into contact with the composition, in order to subject it to the acoustic waves.

The cosmetic composition may also be applied in another way, and in particular continuously, that is to say that a flow of the composition is established in contact with the keratin materials to be treated, this flow being for example in a closed or open circuit.

When the flow takes place in a closed circuit, the composition is recycled at least partially. An additional amount of composition may be introduced continuously or intermittently into the circuit in order to mitigate losses.

At any time, the flow may be stopped to promote prolonged static contact of the composition on the surface to be cleaned. The flow may take place without the presence of bubbles, allowing initial contact with the keratin material to prepare it for cleaning, for example.

In an open circuit, the composition is not recycled to implement the method, and is for example siphoned off to a collection container or directly with wastewater.

The flow of the composition in the device takes place for example at a flow rate of between 0.01 mL per second and 15 mL per second.

The bubbles may be formed while the cosmetic composition is already in contact with the keratin materials, under the action of the acoustic waves and/or using a specific bubble generator of the device, operating for example by electrolysis.

### Selection of the Treated Areas

The method may be implemented on all or part of the facial skin, the scalp or the body in order to clean it.

The treated area may in particular be an area of keratin materials covered with a make-up product such as a foundation, a lipstick, a blusher, a mascara, an eyeliner, a powder, an emulsion, an oil or a sunscreen product, among other things, and the treatment may be aimed at removing this product.

The method may be implemented by moving a handpiece along the area to be treated, so as to treat the entire area covered with the make-up product or other substance that it is desired to remove.

The method may also be implemented to treat an area of skin not covered with a make-up product, in order to perform deep cleaning thereof, and remove or treat exogenous or endogenous impurities or defects, for example dead skin cells, traces of sebum or sweat, dandruff, bacteria, traces of pollution, blackheads, pigmented spots, small scars or acne scars. The method may be implemented to treat the scalp. The method may also be implemented on the nails to remove a nail varnish.

The method may comprise a step of detecting areas to be treated, certain parameters such as acoustic intensity being able to be adjusted automatically according to the requirements in terms of cleaning or treatment for the detected areas.

The device may comprise an optical detector for detecting the presence of a compound to be removed from the surface of the skin, for example a foundation or a sunscreen. The detector comprises for example a light emitter and a sensor for sensing the reflected signal, which may be analyzed to detect the presence of the compound to be removed, and possibly determine the amount present. For example, the emission power of the acoustic waves is adjusted automatically according to the intensity absorbed at a predefined wavelength.

The detection may also be performed in the liquid coming from the treated area, in particular if said liquid is recycled; a high cloudiness of this liquid may be representative of the presence of a large number of particles removed from the surface. The intensity of the acoustic waves may thus be adjusted automatically according to the transparency of the liquid, the intensity being for example automatically increased when the liquid is cloudy, as a large number of particles are removed by the treatment, and reduced when it becomes clear again, a sign that the treatment is no longer removing much.

The method may also be implemented to clean the hair, in particular with a view to at least partially removing a dye applied previously.

The method may also be implemented on the hair in order to remove excess sebum or other substances such as pathogenic or non-pathogenic biological agents and dandruff.

### Additional Methods

The method according to the invention may be preceded or followed by a cosmetic method such as make-up or massage.

For example, the skin, the hair or the eyelashes are made up, and then the make-up is removed after a certain duration (for example less than 24 h) by way of the cleaning method according to the invention.

In another example, the skin is cleaned by implementing the method according to the invention, and then care is carried out on the cleaned area (for example just after or less than two hours after), for example a massage and/or applying a care composition.

### Treatment Kit

Another subject of the invention is a treatment kit for implementing the method according to the invention as defined above.

This kit comprises:
the cosmetic composition in which the bubbles are generated,
a device for exposing the bubbles to acoustic waves in the vicinity of the surface to be treated.

The composition may or may not be packaged with the device in the same packaging.

The composition may result from dilution of a concentrated composition. This dilution may be carried out with a liquid or solid composition, for example in the form of tablets, bars, shavings, powder, preferably a liquid composition, containing all or some of the constituents of the concentrated composition (in different proportions).

Where applicable, the composition is contained in a container designed to be mounted on the device, this container constituting for example a cartridge designed to be fixed fully or partly in a corresponding recess of the device, or be connected to the device by an appropriate link, such as a pipe.

### Other Compounds

The composition may generally comprise any compound conventionally found in the formulation of cleaning and/or care compositions for human keratin materials, compatible with the generation of sufficiently stable bubbles before the acoustic waves are applied.

### Device for Treating Keratin Materials

Another subject of the invention, according to another of its aspects, is a device for treating keratin materials in contact with a cosmetic composition containing at least one surfactant, in particular for implementing the method according to the first aspect of the invention described above, the device comprising at least one ultrasonic transducer designed to emit acoustic waves into the vicinity of said materials to be treated, the transducer being powered by a current generator delivering a pulsed or non-pulsed electrical signal to the transducer.

The device may in particular comprise at least one ultrasonic transducer designed to emit acoustic waves into a fluid present in the vicinity of said materials to be cleaned, the transducer being powered by a current generator delivering a pulsed or non-pulsed electrical signal thereto, this electrical signal preferably having:
at least one frequency component of between 20 kHz and 100 kHz,
a duty cycle Ton/Toff, when the signal is pulsed, of between 20 and 100%, and/or
a pulse duration Ton, when the signal is pulsed, of between 0.01 and 1 s,
the acoustic intensity Isata on the surface of said keratin materials preferably being at least equal to 0.1 W/cm².

The features of the method described above are applicable to the device in combination or independently of one another.

### Device for Treating the Hair

Another subject of the invention, according to another of its aspects, is a device for treating the human hair, in particular with a view to cleaning the hair, removing a previous dye, and/or bleaching said hair, this device being intended to treat the hair while said hair is in contact with a fluid, in particular an aqueous fluid, in particular a cosmetic composition, within which bubbles of a gas are present and/or generated, this device comprising:
at least one transducer having an emission surface for emitting acoustic waves into the vicinity of the hair to be treated, in order to generate bursting of said bubbles,
at least one guiding and/or combing member designed to guide the hair in its movement close to and/or in contact with said emission surface.

The acoustic waves emitted by the emission surface of the transducer make it possible in particular to bring about, by cavitation, the formation and the bursting of bubbles within the fluid, and thus to generate a mechanical effect on the hair that promotes the detachment of exogenous or endogenous elements, and in particular leads to said hair being bleached.

The cosmetic composition may contain very small quantities of surfactants, for example less than 5% surfactants. This small amount of surfactants may prove particularly favorable for the action of the acoustic waves on the fluid and the bubbles present therein.

Among the undesirable impurities that may be present on the hair, it is possible to distinguish between exogenous impurities such as environmental pollution, dust, microorganisms, etc., and endogenous impurities or defects such as excess sebum, dead cells, etc.

The cosmetic composition may or may not have bubbles already present inside it before the acoustic waves are applied.

The bubbles that are generated and/or present may also contribute to the release of chemical species that contribute to the cleaning and/or to the bleaching of the hair, such as free radicals.

The device according to the invention is very particularly suitable for bleaching the hair, in particular hair that has already been subjected to a dyeing treatment, and having a color that is not its natural color.

The device may make it possible to bleach a strand of hair only on the outside, so as to give the hair a swept effect.

The device may also make it possible to bleach specific areas of the hair, in particular specific areas of a strand of hair, through intermittent emission of ultrasound.

The device, and in particular at least part of the guiding and/or combing member, may heat and/or diffuse light, this making it possible for example to activate certain compounds present in the fluid, in particular in the cosmetic composition.

Device with Pump and Tanks

The device according to the invention preferably comprises a system for distributing and/or recovering the composition, comprising at least one composition tank and at least one pump, which is in particular electrically driven, designed to channel the composition in a fluidic circuit between the tank and the keratin materials to be treated.

Another subject of the invention is thus a device for treating keratin materials in contact with a fluid, in particular a cosmetic composition, the device comprising:
a handpiece comprising a treatment head to be placed in contact with the keratin materials to be treated,
carried by the handpiece, at least one ultrasonic transducer designed to emit acoustic waves into the vicinity of said materials to be treated and generate bubbles in the fluid, the bubbles preferably being generated exclusively by the transducer, without an additional bubble generator, and
a system for distributing and/or recovering the fluid, comprising at least one fluid tank and at least one pump designed to channel the fluid in a fluidic circuit between the one or more tanks and the keratin materials to be treated.

Preferably, the device is intended for non-therapeutic use, in particular for the topical application of a cosmetic composition.

The device according to the invention makes it possible to effectively treat both exogenous impurities and endogenous impurities or defects, while still meeting certain environmental demands and applicable safety standards.

The system for distributing and/or recovering the fluid makes it possible to save on water and chemical compounds.

Preferably, the handpiece comprises a handle, which may be arranged non-coaxially with the treatment head. The handle may at least partially contain the system for distributing and/or recovering the fluid. As a variant, the device may comprise a base station connected to the handpiece by a cord, the pump and/or the one or more tanks being arranged in the base station.

### Pump

The pump may have any type of drive, in particular a manual or electric drive, preferably an electric drive.

The pump may be of various types. The pump is for example a volumetric or centrifugal pump, in particular a peristaltic pump, membrane pump, piston pump or a combination of several types.

The pump may be powered by a DC or AC current. Preferably, the supply voltage for the pump is less than 50 V, being for example powered with a voltage ranging from 5 to 12 V.

The pump is preferably compatible with liquids of varied nature, in particular aqueous, oily and mixed liquids, liquids comprising particles or fillers in suspension, or even polymers.

When the pump is driven by an electric motor, the motor is for example a brushed or brushless DC motor, in particular with permanent magnets. These magnets may be based on ferrites or rare earths.

The motor has for example, when used to drive the pump, a rotational speed of between 1 and 10 000 rpm.

Such a speed may be fixed, or variable, the motor being for example driven by a variable speed drive, for example with pulse width modulation, with (passive or active) resistance acting on the current or the supply voltage, or by a variable frequency drive.

The motor may be single-phase or three-phase.

The mechanism of the pump may be driven directly by the motor or using a reducer.

When the drive is manual, the device may comprise a handle that is actuated by the user to operate the pump.

Preferably, the pump is configured to provide a fluid flow rate between 0.01 mL and 15 mL per second.

The pump may or may not be removable. The pump may generate a negative and/or positive pressure on the fluid in order to allow the fluid to flow and to put it into contact with the skin. It is also possible to use the pump for the internal cleaning of all or part of the device.

The pump may contain a filter in one or more parts. The pump may be mechanically or electrically isolated in the device, for example in order to be removed from the device for cleaning.

### Tanks

The device may comprise one or more fluid tanks, this fluid preferably being the abovementioned cosmetic composition.

The one or more tanks may be arranged in the handpiece, in particular in its handle, or at least partially outside the handpiece, for example in a base station connected to the handpiece.

As a variant, the one or more tanks may be arranged so as to project from the handpiece.

The device may comprise multiple separate tanks, for example a tank for fluid to be distributed over the area to be treated and a tank for receiving used fluid that has been at least partially recovered following the treatment.

The one or more tanks may or may not be removable. The one or more tanks are for example single-use tanks, and may be replaced when there is no more fluid, or the recovered fluid needs to be drained.

It is also possible to provide tanks of varied capacity depending on the treatment to be performed on the keratin materials, the device being able to be configured to allow the user to install the tank of their choice, chosen from among multiple different respective capacities.

The one or more tanks may be cleaned, filled or emptied, for example manually from the outside by being removed from the device beforehand, or else directly when they are still present in the device, for example using the pump and the fluidic circuit.

In this case, the device comprises for example a selector that makes it possible to change the flow of fluid from a first configuration, in which the flow takes place between the tanks and the treatment area, and a second configuration used to purge and/or to fill the tank.

The one or more tanks may comprise multiple compartments, in particular multiple compartments integrated into one and the same body and separated by a wall, or multiple communicating compartments, for example connected by a tube. The one or more tanks may comprise a compartment containing the composition to be distributed over the areas to be treated, and a compartment collecting the used composition, with a view for example to recycling it. The two compartments may communicate, via a filter for example.

The one or more tanks may also comprise multiple compartments in which different compositions may be stored. These compositions may be mixed when they are used and may in some cases be solid and/or in the form of rapidly soluble powders.

For example, one tank may contain a first compartment containing a solid composition, for example in the form of powder or crystals, and a second compartment containing a fluid composition, the two compartments being able to communicate during use so that the fluid composition solubilizes the solid composition.

The first compartment may also contain a mixture of a suspended solid composition and a liquid in which the solid composition is insoluble, the fluid composition in the second compartment solubilizing the mixture during use of the device.

The one or more tanks may comprise one or more filters for filtering impurities in the collected fluid before redistributing it over the area to be treated. The tank may also comprise a vent.

The one or more tanks may comprise a device for heating or cooling the fluid, in particular an electrical resistor.

The one or more tanks are preferably compatible with one or more liquids of varied nature, in particular various types of miscible or immiscible composition and of cleaning liquids.

The one or more tanks are preferably at least partially transparent, thereby allowing the user to visually observe their fill level and/or the level of cleanliness of the fluids that they contain.

The one or more tanks may be deformable or made of rigid material, in particular of glass or of plastic. A tank may comprise a removable part, and thus have an adjustable volume according to what is desired. The one or more tanks may be fitted with valves allowing them to be decoupled from the housing receiving them without loss of fluid.

The one or more tanks may be in the form of cartridges that are generally cylindrical or of another shape, or flexible pockets.

The capacity of the one or more tanks is for example between 1 ml and 200 ml, better still between 1 ml and 50 ml.

### Fluidic Circuit

The fluidic circuit preferably comprises a plurality of ducts, which are connected appropriately so as to produce the desired fluidic flow, for example with end-to-end or parallel connections, connected together by appropriate connections.

The fluidic circuit connects the various parts of the device involved in the flow of the fluid together, such as the treatment head, the one or more pumps or the one or more fluid tanks, along with the one or more filters or other purification or disinfection or even additional bubble generation systems.

The fluidic circuit may comprise one or more modifiable taps or valves, for example one or more manual or electrically controlled valves.

The pump may contain a filter in one or more parts.

The fluidic circuit may comprise a water intake for filling it with water and/or for cleaning it from an external network.

The fluidic circuit may also be able to be modulated with removable sections, for example sections that are added when cleaning the circuit. It is possible for example to add a section comprising a cleaning powder that solubilizes at least partially when a liquid passes through the circuit. The section that is added may thus be used only for cleaning, and removed afterwards, or be left in place until the next cleaning operation, then being replaced with a new one.

The one or more tanks and the fluidic circuit allowing the fluid to flow may belong to one and the same assembly forming a refill, capable of being manipulated in one piece in order to install it on the device and remove it therefrom; refilling with product is thereby made easier. Such a refill may where applicable have a mechanical interface with the pump or other drive mechanism, making it possible, when actuated, to establish a flow of product from the cartridge to the treatment area.

The assembly forming a refill may be arranged so as to be fastened, for example by a snap fit or the like, in the handpiece, for example in one and the same housing as the one containing another component of the device, for example an electrical generator.

### Treatment and Purification Unit for the Fluid

The device according to the invention may comprise a system for at least partially recycling the fluid, comprising a duct opening out close to the area to be treated and communicating with a suction system, in particular a suctioning pump.

The suction system is preferably designed to send the recovered fluid to a treatment and purification unit, the fluid being for example returned, at the output of the treatment and purification unit, to a tank in order to be distributed over the area to be treated again.

The treatment and purification unit may be arranged inside the handpiece, in particular in its handle, or be at least partially transferred to the outside, for example to a base station connected to the handpiece by a cord.

The treatment and purification unit preferably comprises one or more removable parts. It may be washable and fully or partly reusable.

The treatment and purification unit may comprise a filtration system capable of at least partially filtering any fluid flowing in the device, in particular the cosmetic composition used for the treatment, or any additional liquid used to clean the device.

Preferably, the filtration system comprises at least one filter, in particular a filter having a pore size of less than or equal to 50 microns.

The filter may be a nonwoven, with or without folds. The filter may comprise one or more elements chosen from among a fabric, a porous material, active carbon particles, sand, silica, a porous polymer, a natural or synthetic foam.

The filter may be a filter with a high capture capability, in particular a nanofilter.

The filtration system may also comprise a prefilter, which may be positioned downstream or upstream of the suction system.

The filtration system may comprise a sensor for sensing the efficiency of the one or more filters, configured to indicate to the user whether a filter should be changed or cleaned, for example by changing color when the filtration system is saturated. Such a sensor may also be a pressure sensor able to detect clogging of the filter.

Where applicable, the fluidic circuit of the device is designed to channel the fluid in counter-current during a cleaning phase in order to unclog the filter.

The treatment and purification unit may comprise multiple elements distributed in various parts of the device; for example, this unit may comprise a filtration system present in at least one duct of the fluidic circuit, and a disinfection system or a system aimed at carrying out another treatment, arranged in a compartment of a tank.

The treatment and purification unit may comprise a centrifugal separator designed, using the centrifugal force imparted on the fluid by making it swirl, to extract impurities from used fluid that has been in contact with the keratin materials to a collection area.

The treatment and purification unit may comprise a disinfection unit that destroys any living organisms present in the recycled fluid, such as bacteria, viruses, spores or other pathogenic or non-pathogenic biological agents.

The disinfection unit may to this end comprise any appropriate biocidal means, preferably at least one UV lamp and/or an oxidizer or ozone generator, in particular a UVC LED lamp.

### Ultrasonic Transducer

The device comprises an ultrasonic transducer.

As described above, the transducer comprises one or more electroactive elements for converting an electric current into mechanical vibrations, these one or more electroactive elements advantageously being able to be coupled to a sonotrode that is set in mechanical resonance and defines an emission surface for emitting acoustic waves toward the surface or the target volume.

These acoustic waves may bring about rapid pressure changes that may lead to the cavitation of bubbles; the acoustic waves may, as described above, lead to the formation and bursting of bubbles, or to the bursting of bubbles already present in the medium.

The longitudinal axis of the transducer may designate that of the sonotrode. The latter may be rotationally symmetrical about its longitudinal axis, or of another shape. This may be an axis of symmetry of the transducer, this being for example rotationally symmetrical about said axis or of another shape.

The longitudinal axis may also correspond to the direction in which the one or more electroactive elements have the greatest movement amplitude when excited.

The sonotrode may be made of metal, preferably of titanium, thereby improving the effects of the cavitation of the bubbles. The sonotrode may also be at least partially made of aluminum, stainless steel, or of ceramic. The sonotrode may comprise multiple metals or alloys.

The transducer may comprise at least one piezoelectric material, in particular lead zirconate titanate (PZT).

Preferably, the sonotrode is made of a corrosion-resistant material that allows it to be at least partially submerged in the composition.

The emission surface for emitting acoustic waves of the sonotrode may be covered with a protective layer for protecting the sonotrode from any degradation due to the cavitation of the bubbles in contact therewith, and/or from corrosion.

The invention is not limited to a particular shape or to a given size for the sonotrode. However, preference is given to a size compatible with the portable nature of the handpiece, in order to allow it to be manipulated by the user using one hand.

The sonotrode may have an emission surface in contact with the composition that ranges for example from the surface area of a circle with a diameter of 5 mm to a circle with a diameter of 100 mm, better still from the surface area of a circle with a diameter of 5 mm to that of a circle of 50 mm, better still from the surface area of a circle with a diameter of 5 mm to that of a circle of 40 mm.

The sonotrode may be given any shape tailored to that of the surface to be treated, for example round, oval, or polygonal, in particular square or triangular.

The emission surface of the transducer may be removable with respect to the transducer, for example so as to be able to be replaced by the user, in particular in order to offer various solutions for guiding and/or combing the hair and/or for adapting to the type of relief of the guiding and/or combing member.

Preferably, the entire transducer or the sonotrode are removable, that is to say that they are able to be detached easily by the user from the device, preferably without involving any tools. This facilitates cleaning thereof and makes it possible, if desired, to interchange multiple transducers or sonotrodes depending on the desired use and energy parameters, for example by choosing the type of transducer or sonotrode best suited to generating acoustic waves at a given frequency.

Interchanging the transducers makes it possible to vary the acoustic intensity as required.

The device may thus be used for a wide choice of applications, depending on the transducer and/or the sonotrode chosen. The choice of the transducer and/or of the sonotrode may depend on various parameters, for example the nature of the desired treatment, the type of keratin materials to be treated, or even the frequency of use of the device (daily, weekly, etc.).

The device may also comprise an adjustable-opening mechanical element, such as a diaphragm, arranged in front of the sonotrode so as to limit the emission surface for emitting ultrasonic waves. The diaphragm may be adjustable by the user in order to adjust the power of the treatment and/or the size of the treatment area.

The transducer and/or the sonotrode may comprise fastening means for fastening to the rest of the device by snap fitting, by screwing, by bayonet, by friction, magnetically, by clamping, in particular using at least one clamping screw, a hose clamp, a magnet, or by any other appropriate means.

The transducer may move longitudinally and/or transversely, in particular in order to move toward or away from the hair to be treated. It is able to rotate about its longitudinal axis and/or vibrate.

The sonotrode may have any shape at the emission surface for emitting acoustic waves.

The device may comprise drive means for driving the transducer or the sonotrode, configured to generate an oscillating, vibrating and/or rotational movement thereof, independently of the acoustic waves that are generated.

The transducer may be powered with DC or AC, in particular independently from the other elements of the device, such as the pump.

The transducer or the associated sonotrode may comprise a duct opening out preferably on the emission surface, connected to the fluidic circuit such that they are flowed through by the fluid flowing in the device. Such a flow makes it possible to cool the transducer.

The device may comprise multiple transducers, in particular two transducers. This makes it possible for example to increase the effectiveness of the treatment by the device.

### Reliefs on the Emission Surface

The emission surface for emitting acoustic waves toward the liquid medium may comprise reliefs where the bubbles may arise at different levels along the longitudinal axis of the transducer. In particular, these reliefs may offer nucleation sites at their peak and at their base.

Another subject of the invention, according to another of its aspects, is thus a device for treating keratin materials in contact with a fluid, in particular a cosmetic composition, the device comprising:
At least one ultrasonic transducer having an emission surface for emitting acoustic waves into said fluid in order to generate bubbles therein having a mechanical action, when they burst, on said keratin materials, the emission surface having reliefs where the bubbles may arise at different levels along the longitudinal axis of the transducer.

The reliefs may allow more homogeneous production of bubbles. In comparison, a smooth emission surface tends to generate nucleation sites that are distributed randomly over the surface, and non-optimum bubble production. These reliefs may also promote the bursting of the bubbles, since the different levels along the longitudinal axis make it possible to generate relatively large cavitation areas, not limited to a single plane.

The reliefs may also make it possible to generate a turbulent flow in the fluid, which may contribute to the production of bubbles, in particular when the fluid contains a surfactant.

The reliefs may be formed in various ways.

The reliefs may be produced using any method that makes it possible to create a predefined shape by removal of material, for example by machining, chemical attack, electroerosion, laser etching, molding or additive synthesis, or by inlaying foreign bodies.

The reliefs may be monolithic with the sonotrode, for example being produced by machining said sonotrode.

The reliefs may also be formed on at least one added piece that is fastened to the sonotrode.

This added piece receives at least some of the vibrations of the sonotrode and transmits them to the liquid medium; it defines at least part of the emission surface for emitting acoustic waves into the fluid.

The added piece may be made of the same material as the sonotrode, or of a different material, in particular a harder material. The added piece that comprises the reliefs is preferably made of a rigid material in order to transmit the acoustic waves without excessive attenuation. It may be made of metal, of an alloy or of a ceramic.

Using an added piece to define the emission surface for emitting acoustic waves into the liquid medium may facilitate replacement thereof in the event of wear of the emission surface, the bursting of the bubbles being liable to lead to wear thereof.

The added piece may be fastened to the sonotrode by any means, in particular held thereon by a removable fastening. For example, the added piece is screwed to the sonotrode. When the fastening does not have to be removable, the added piece may be welded or otherwise fastened to the sonotrode.

The sonotrode may be made of a material harder than aluminum, for example titanium or stainless steel.

The reliefs may thus be made of titanium with the sonotrode, for example by machining the end face thereof.

The added piece may be made of a material harder than aluminum, for example titanium or stainless steel. In this case, the sonotrode may be made of titanium or of a material less hard than titanium, for example aluminum, since it is not exposed directly to cavitation.

The reliefs preferably have a predefined shape but, as a variant, they may be created randomly, and have for example at least one parameter that is random, for example their size or their location. All or some of the reliefs may thus have a random distribution over the emission surface and/or a random size.

The reliefs may be arranged in a regular arrangement, in other words a structured shape, for example in a regular array in two dimensions (the third one being the longitudinal axis of the sonotrode).

The reliefs may be identical; for example, the emission surface comprises one and the same elementary pattern that repeats in two mutually perpendicular directions.

This pattern may have a prismatic shape, for example a pyramidal shape or the like, for example a hemispherical, cylindrical, hemicylindrical, ogival, etc. shape.

The reliefs may be in the form of ribs or ridges, where applicable, which may or may not be parallel with one another, rectilinear or circular, or in a grid shape, or have a random distribution. The ridges may be formed by scratching the surface for example. The reliefs may be formed by subjecting the surface to shot-blasting or sanding, which may lead to a random distribution and/or to random shapes of reliefs.

The emission surface may have reliefs having a height, measured parallel to the longitudinal axis of the sonotrode, of between 0.001 mm and 50 mm, better still 0.01 mm and 30 mm, even better still between 0.1 mm and 1 mm.

In a front-on view, that is to say when the emission surface is observed in the longitudinal axis of the sonotrode, the largest dimension of the reliefs may be between 0.001 mm and 100 mm, better still between 0.1 mm and 1 mm.

The free end of the reliefs may be rounded or flat in order to avoid injuring the skin in the event of contact therewith.

The emission surface for emitting acoustic waves may thus comprise at least one relief having a pyramidal, frustoconical, cylindrical, hemispherical or prismatic irregular shape. The emission surface may comprise a regular array of four-faced pyramidal reliefs.

The emission surface for emitting acoustic waves may comprise at least one relief having at least two facets oriented differently toward the area to be treated.

The reliefs may where applicable have another additional function, for example of guiding and/or combing hair in the treatment area.

When the reliefs are formed by the presence of successive patterns in one or two directions, the size of the patterns and the distance between consecutive patterns are for example chosen such that the pattern density is between 1 and 10⁶ patterns/cm².

When the patterns have peaks, all of the peaks belong for example to one and the same plane or to one and the same spherical, cylindrical, parabolic or ellipsoidal surface.

In addition to the reliefs of the emission surface that contribute to improving the production of bubbles, the emission surface may have reliefs that pursue another aim, for example exerting an action of retaining fluid or of cleaning the area to be treated. These are for example reliefs made of a flexible material, for example bristles, in particular flocking bristles.

The emission surface of the transducer may have one or more reliefs, such as teeth, and/or natural and/or synthetic bristles, and/or etchings, and/or micro-projections, and/or metal spikes, so as for example to comb and/or to contribute to guiding the hair, upstream or downstream of the treatment area, or even in said treatment area.

The emission surface may have the same type of reliefs as the guiding and/or combing member or, as a variant, different reliefs.

The emission surface may also have one or more reliefs when the guiding and/or combing member does not have any. As a variant, the guiding and/or combing member may have one or more reliefs when the emission surface does not have any.

### Additional Vibrations

Another subject of the invention is a device for treating keratin materials in contact with a fluid, in particular a cosmetic composition, the device comprising:
At least one ultrasonic transducer having an emission surface for emitting acoustic waves into said fluid in order to generate bubbles therein having a mechanical action, when they burst, on said keratin materials,
a vibrator for subjecting at least part of the emission surface to additional vibrations of a frequency lower than that of the acoustic waves.

It is possible to subject at least part of the emission surface to additional vibrations of a frequency lower than that of the acoustic waves, for example vibrations of a frequency less than or equal to 1500 Hz, even better still less than or equal to 150 Hz, or even 50 Hz.

The whole device may be subjected to said additional vibrations. As a variant, only part is subjected to them, for example only the transducer, the sonotrode or part thereof, or the treatment head.

The device may to this end comprise a vibrator, for example a vibrator comprising a motor driving an imbalance in rotation.

The emission surface is thus subjected by the vibrator for example to additional transverse and/or longitudinal and/or angular, preferably longitudinal, vibrations. It is possible to adjust the direction of the vibrations for example by acting on the orientation of the axis of rotation of the imbalance relative to the longitudinal axis of the transducer.

These additional vibrations may promote greater homogeneity of the cavitation and may improve the wetting of the emission surface for emitting acoustic waves by the fluid.

The additional vibrations may be emitted at the same time as the emission of the acoustic waves or as a variant alternately; the device may be designed, where applicable, to make it possible to activate or not activate the generation of these vibrations, for example by starting or not starting the motor driving the imbalance, or even to adjust its rotational speed.

It may be particularly advantageous to subject the emission surface to these additional vibrations in the presence of reliefs as defined above on the emission surface, the association of the reliefs and the vibrations making it possible to more easily obtain additional turbulence in the fluid in contact with the materials to be treated, thereby improving, as described above, the production and bursting of bubbles within the fluid.

### Treatment Head

The device may comprise a treatment head in contact with the keratin materials to be treated, the head preferably being designed to distribute the fluid over the area to be treated via at least one fluid outlet, and/or at least partially recover the used fluid from the area to be treated via at least one fluid return inlet.

The device for treating the hair may comprise a treatment head in which the fluid, in particular the cosmetic composition, flows, the transducer and the guiding and/or combing member extending at least partially within this treatment head.

The transducer preferably extends at least partially within the treatment head. It may in particular be housed fully therein.

The outlet and/or the inlet are for example connected to the abovementioned fluidic circuit. In particular, the fluidic circuit may be arranged with respect to the head so as to distribute and/or partially recover the fluid upstream and/or downstream of the emission surface of the sonotrode, for example via at least one orifice on the emission surface.

The treatment head may comprise a heating element such as an electrical resistor for heating the composition for example, and/or a light source arranged so as to illuminate the treatment area during use and/or activate certain constituents of the fluid in which the cavitation occurs.

The treatment head may comprise at least one filter, which is used for example to filter the composition that is recycled, this filter being arranged for example on the return path of the fluid.

The treatment head may comprise a porous material and/or a material able to release or diffuse a fluid, in particular an open-cell foam, preferably carried by a removable support, in particular in the form of a frame.

The treatment head may comprise distribution orifices such as slots, which are closed in the rest state, which open under the pressure of the upstream composition, and have an elasticity that allows an increase in volume during filling and distribution of the fluid that is extended after the end of the filling action.

The treatment head may comprise a chamber for storing a quantity of fluid sufficient to allow cleaning of the keratin materials and allowing bubbles to be put into contact with the surface to be cleaned.

The treatment head may comprise multiple independent or intercommunicating compartments.

The treatment head may comprise parts made of a flexible and/or deformable material, in particular an elastically deformable material, in particular for the parts in contact with the keratin materials to be treated.

The treatment head may be of various shapes, depending on the desired use for the device.

The treatment head may comprise one or more removable parts, able to be detached easily by the user from the device, preferably without involving any tools.

This facilitates cleaning thereof between uses.

The one or more removable parts of the treatment head may be fastened to the rest of the device via any appropriate fastening means, in particular a ball joint, spring, or a piston. The treatment head is preferably mobile; in some examples, it is able to tilt or be rotated over more than 180°, 270° or 360° about at least one axis.

The transducer is preferably arranged with respect to the treatment head so as to keep the emission surface for emitting acoustic waves of the sonotrode at a distance from the surface of the keratin materials to be treated.

Where applicable, the transducer is able to move relative to a fixed part of the device, for example driven in a rotational or to-and-fro movement, as described elsewhere.

The cavitation thus occurs in the front part of the head, in a space formed between the emission surface of the sonotrode and the surface of the keratin materials to be treated.

The fluid may thus be made to flow in this space, which may be able to be modulated.

This flow may take place continuously, as mentioned above, or intermittently. It is also possible to fill said space with the fluid while the handpiece is in place, and then generate the acoustic waves within the fluid filling this space.

Guiding and/or Combing Member

In the case of a hair treatment, the device according to the invention may comprise at least one guiding and/or combing member designed to guide the hair in its movement close to and/or in contact with the emission surface of the transducer.

The guiding and/or combing member makes it possible to ensure that the hair is located at an appropriate distance from the emission surface for emitting acoustic waves during the treatment, and thus to maintain the effectiveness of the treatment.

The guiding and/or combing member may keep the hair at a predefined distance from the emission surface, for example having the role of the spacer.

The guiding and/or combing member may keep the hair in the vicinity of the emission surface, by stopping the hair from moving away in a direction parallel to the longitudinal axis of the transducer or in a direction both perpendicular to the longitudinal axis of the transducer and the longitudinal direction of the hair; the guiding and combing member does not stop the hair moving through in front of the emission surface for emitting acoustic waves.

The guiding and/or combing member might not stop the hair coming into contact with the emission surface for emitting acoustic waves in the absence of a spacer placed in front thereof.

The guiding and/or combing member also allows a good division of a strand of hair and makes it possible to obtain, in the treatment area, a homogeneous thickness of the hair, thereby allowing homogeneous treatment of the strand.

In some examples, the hair is combed by said guiding and/or combing member, thereby potentially making it possible to spread the hair in front of the emission surface in order to facilitate the action of the bubbles.

The guiding and/or combing member may be designed to laterally guide the hair, for example by having stops between which the hair is engaged.

The guiding and/or combing member may comprise at least one surface for laterally guiding the hair, making it possible to hold the hair during treatment thereof.

At least one lateral guiding surface, better still two lateral guiding surfaces, of the guiding and/or combing member may be perpendicular to the emission surface for emitting acoustic waves.

The guiding and/or combing member may be made at least partially of a metal material and/or a polymer, for example a rigid plastic.

The guiding and/or combing member may have, on its surface, a treatment that makes it possible to reflect the acoustic wave, thereby making it possible to amplify the treatment of the hair.

The guiding and/or combing member may be fastened removably or non-removably to the device.

At least part of the guiding and/or combing member may vibrate and/or rotate and/or move longitudinally and/or transversely.

The guiding and/or combing member may be in the form of a single-piece element or of multiple elements joined to one another or to the device in order to carry out the one or more desired guiding and/or combing functions.

The guiding and/or combing member may comprise at least one guiding portion intended to come into contact with the hair and at least one support portion that serves to keep the guiding portion in the desired configuration during the treatment.

Guiding Portion for the Guiding and/or Combing Member

The guiding portion may be fixed or mobile with respect to the rest of the device, and may in particular be mobile with respect to the transducer, for example in order to bring it closer to or away from the emission surface for emitting acoustic waves. It is thus possible to control the distance between the emission surface and the surface of the hair to be treated relatively precisely, thereby making it possible to improve the action of the bubbles in the vicinity of the surface of the hair, while still avoiding, if desired, direct contact between the emission surface and the hair.

When it is mobile, the guiding portion may be rotational or axially mobile, for example rotate about its own longitudinal axis and/or move along its own longitudinal axis and/or move along an axis perpendicular to its own longitudinal axis.

The guiding portion may have a guiding surface extending at least partially facing the emission surface and/or on either side thereof, in particular defining a treatment space with the emission surface.

The distance between the emission surface and this guiding surface may be between 0.1 mm and 50 mm, in particular between 0.1 mm and 30 mm, where applicable adjustable by the user, in particular in order to adapt the device to the thickness of hair to be treated.

The guiding surface may have a width greater than or equal to that of the emission surface, for example have a width of between 1 mm and 180 mm, the emission surface having for example a width of between 1 and 150 mm. “Width” here designates the dimension in a direction perpendicular to the direction of travel of the hair, and perpendicular to the longitudinal axis of the transducer.

The guiding portion may be hollow so as to define a cavity for guiding the hair to be treated, the cavity defining a treatment space. This cavity may be open at two opposing ends and the hair may extend between these two ends. The guiding portion may be moved along a strand of hair to be treated, the hair moving in the cavity between its ends.

The cavity may be laterally open between its ends in order to allow the hair to be inserted. As a variant, the hair is inserted via one of the ends before leaving via the other end.

The cavity may be formed by a guiding portion of generally tubular shape, in particular cylindrical shape, in particular a tubular shape with axial slots.

The inner surface of the cavity may have reliefs that contribute to combing and/or holding the hair. These reliefs may be in the form of ridges for example, or have another shape, as described further below.

The guiding portion may be at least partially permeable to the acoustic waves and the transducer placed outside the guiding portion, such that the acoustic waves emitted by the emission surface of the transducer reach the fluid, in particular the cosmetic composition, present in the cavity with the hair, generate bubbles and/or cause them to burst.

The guiding portion may also form all or part of the transducer, in particular all or part of a sonotrode thereof, while still defining the abovementioned cavity, the emission surface in this case being formed by at least part of the surface of the cavity.

The cavity may have a constant or variable inside diameter. “Inside diameter” designates that of the largest circle inscribed within the cross section of the cavity.

The guiding portion may be located between at least two transducers, for example two diametrically opposing transducers.

For example, the guiding portion defines a cavity for guiding hair that is located between two transducers; this cavity is for example open at its axial ends between one another so as to define a slot for inserting the hair.

The guiding portion may be in contact with the two transducers, forming for example all or part of the sonotrode thereof, and/or be permeable to the acoustic waves emitted thereby.

The guiding portion may comprise at least two parts that are mobile with respect to one another, for example in the form of jaws, between a spaced configuration for inserting hair between them, and a close configuration for keeping the hair in the treatment space.

The guiding portion comprises for example a proximal part, closer to the transducer, and a distal part that is further away.

The proximal part may be permeable to the acoustic waves and may overlap at least partially, in a front-on view, with the emission surface. “Front-on view” designates the view in a direction generally perpendicular to the emission surface.

The proximal part of the guiding portion, in order to be permeable to the acoustic waves, may comprise holes, in particular be produced in the form of a grating, or else be made of a specific material permeable to the acoustic waves.

The distance between the proximal and distal parts may vary between 0 mm in the close configuration and 100 mm in the spaced configuration, in particular between 0 mm in the close configuration and 50 mm in the spaced configuration.

The proximal part may be fixed relative to the transducer or mobile with respect thereto.

The distal part may be fixed relative to the transducer or mobile with respect thereto, provided that at least one of the parts is also mobile with respect to the other.

At least one of the distal and proximal parts may be urged to move to a rest position by at least one elastic return means, such as a spring.

In particular, the proximal and distal parts may be returned, by one or more elastic return means, to the close configuration or, as a variant, to the spaced configuration, and are preferably also configured to allow the user to exert a force opposite to the return action, in order to move the proximal and distal parts in the opposite direction when necessary, for example when inserting the hair between said parts, when removing the hair, and/or during the treatment.

The guiding member may comprise a single guiding portion or multiple guiding portions.

Support Portion for the Guiding and/or Combing Member

The guiding and/or combing member may comprise at least one support portion that holds it during use of the device.

The support portion may comprise a mechanism allowing a translational movement of the guiding and/or combing member along the longitudinal axis of the transducer, in particular a mechanism including one or more springs for returning the support portion to a rest position. This rest position corresponds for example to pressing of the hair against the guiding and/or combing member and/or against the emission surface during the treatment.

The support portion may be mobile relative to the transducer in order to clear a space facing the emission surface, and allow for example the hair to be inserted into the treatment area.

The portion may be mobile in translation or rotation or in any kinematics combining at least one translation and one rotation.

For example, the support portion may pivot relative to the transducer about an axis of rotation perpendicular to the longitudinal axis of the transducer.

If the guiding and/or combing member comprises a two-part support portion, these two parts are able to pivot simultaneously in order to switch the guiding and/or combing member on the side of the transducer.

Reliefs on the Guiding and/or Combing Member

The guiding and/or combing member may comprise at least one guiding and/or combing relief on its surface.

The guiding and/or combing relief may extend continuously or discontinuously over the guiding and/or combing member. The guiding and/or combing relief may be absent from some parts of the guiding and/or combing member.

The guiding and/or combing member may comprise, on its surface, at least one guiding and/or combing macro-relief able to engage in the hair, in particular at least one tooth, and/or a natural or synthetic bristle.

The guiding and/or combing member may comprise, on its surface, in particular on the surface of at least one tooth, at least one micro-relief, in particular an etching and/or a micro-projection, resulting in particular from a sanding treatment on the guiding and/or combing member.

These guiding and/or combing reliefs make it possible to guide and/or to comb and/or to hold and/or to tension the hair during treatment.

At least one guiding and/or combing relief, in particular at least one tooth and/or at least one bristle, may extend in a space located facing the emission surface.

The guiding and/or combing member may comprise at least one guiding and/or combing relief, in particular a tooth and/or a bristle, extending outside a space located facing the emission surface. The guiding and/or combing member may in particular comprise at least one row of teeth, or bristles, extending outside a space located facing the emission surface.

The guiding and/or combing member may comprise at least one row of teeth, or bristles, between which the hair is able to engage.

The distance between the longitudinal axes of the teeth, or the bristles, of a row may be between 0.1 mm and 10 mm.

The teeth, or the bristles, of one and the same row may all be aligned. As a variant, the teeth, or the bristles, of one and the same row may be arranged in a quincunx.

At least one tooth may have a conical shape, with a circular or elliptical base. As a variant, at least one tooth may have a pyramidal shape with a square or rectangular base, or a cylindrical shape.

At least one tooth, or one bristle, in particular at least one tooth of a row of teeth, or one bristle of a row of bristles, respectively, may have a height of between 0.1 mm and 50 mm, in particular between 0.5 mm and 20 mm.

At least part of the guiding and/or combing member may be in the form of a brush such as a brush with a twisted core comprising natural and/or synthetic bristles.

At least part of the guiding and/or combing member, for example part of the guiding and/or combing member in the form of a brush such as a brush with a twisted core, may rotate about its longitudinal axis, underneath the emission surface as the hair passes between the guiding and/or combing member and the emission surface.

The guiding and/or combing member may have a single type of guiding and/or combing reliefs or, as a variant, multiple different types of guiding and/or combing reliefs.

Flow of the Fluid in the Guiding and/or Combing Member

At least part of the guiding and/or combing member may be hollow in order to allow the fluid, in particular the cosmetic composition, to flow inside.

At least part of the guiding and/or combing member may be passed through by a duct that may make it possible to supply the fluid, in particular the cosmetic composition, to the treatment space, for example.

In its hollow part, the guiding and/or combing member may receive an anhydrous composition, such as a composition of a carbonate, in particular of sodium or of calcium, and of an acid, citric acid for example, able to solubilize as the fluid, in particular the cosmetic composition, flows, and able to release bubbles of gas.

The guiding and/or combing member may comprise at least one orifice allowing the fluid, in particular the cosmetic composition, flowing inside to exit, this orifice opening out for example in the treatment space. The orifice opens out for example in the direction of the emission surface; for example, it is located facing the emission surface.

Such an orifice may make it possible to release the fluid, in particular the cosmetic composition, directly in contact with the hair, thereby possibly contributing to the effectiveness of the treatment.

The guiding and/or combing member may comprise between 1 and 100 000 orifices or more, allowing the fluid, in particular the cosmetic composition, flowing inside to exit. These orifices may be formed by machining for example or be created by the intrinsic structure of the material that is used.

The guiding and/or combing member may thus be made at least partly of a porous material, having a very large number of pores allowing the fluid, in particular the cosmetic composition, flowing inside to exit.

The guiding and/or combing member may comprise at least one orifice allowing the fluid, in particular the cosmetic composition, to be recovered following treatment, for example by suctioning of the fluid, in particular the cosmetic composition.

The guiding and/or combing member may be at least partially covered by a fabric or nonwoven sleeve; the sleeve may be made at least partially of a polymer so as not to damage the fibers of the hair. The sleeve may be formed so as to allow the fluid, in particular the cosmetic composition, leaving the orifices of the guiding and/or combing member to pass through, and thus allow better diffusion of the fluid, in particular the cosmetic composition.

The sleeve may be at least partially soluble, in particular fully soluble, as the fluid, in particular the cosmetic composition, exits via the orifices, and may thus contribute to the treatment of the hair by virtue of the compounds released when it dissolves. For example, the sleeve may be made at least partially of polyvinyl alcohol (PVA) fibers or any other soluble polymer able to be in the form of a fabric, a foil or even in a nonwoven form.

At least one guiding and/or combing relief may be hollow in order to allow the fluid, in particular the cosmetic composition, to flow inside.

At least one guiding and/or combing relief may comprise at least one orifice allowing the fluid, in particular the cosmetic composition, flowing inside to exit.

### Nozzle

The treatment head may comprise a nozzle, which is preferably removable, in contact with the keratin materials to be treated.

The nozzle, when it is removable, is for example chosen depending on the use, for example by selecting the type, the size, the hardness and/or the shape best suited to the morphology of the area to be cleaned.

### Grating

Another subject of the invention is a device for cleaning human keratin materials in contact with a cosmetic composition within which bubbles of a gas are present and/or generated, the device comprising:
at least one ultrasonic transducer designed to emit acoustic waves into the vicinity of said materials to be cleaned,
a grating, which is preferably non-absorbent, in particular at least partially made of a non-absorbent material, defining a contact surface with said materials to be cleaned, having at least one opening, preferably at least two openings, through which the waves emitted by the transducer are able to propagate in the direction of the materials to be cleaned.

By virtue of the grating, it is possible to control the distance between the transducer and the surface of said keratin materials more precisely, thereby making it possible to improve the action of the bubbles in the vicinity of the surface of the keratin materials, while still avoiding direct contact between the transducer and these keratin materials.

The grating is preferably non-absorbent, that is to say that it is not porous.

The grating may in particular not have the capacity to absorb water when it is dipped in water, and retain for example less than 10% water, better still less than 5% of its weight by water.

The grating may in particular be made of a solid material, therefore other than a foam, a fabric or a felt.

The contact surface of the device with the keratin materials may be defined by a non-absorbent surface of the grating.

The region of the grating defining the openings therein may be defined by a non-absorbent part of the grating.

The grating is preferably removable, that is to say that it is able to be detached easily by the user from the device, preferably without involving any tools. This facilitates cleaning thereof and makes it possible to replace it between uses, or even, if desired, to interchange multiple gratings depending on the use, for example by choosing the type of grating best suited to the morphology of the area to be cleaned.

The contact surface defined by the grating may be substantially flat, in order for example to maintain a substantially constant distance between the transducer and the keratin materials to be cleaned located facing the emission surface for emitting acoustic waves.

As a variant, the grating may define a contact surface tailored to the morphology of the area to be cleaned, for example of curved shape.

The grating may furthermore have, where applicable, an adjustable effective diameter. The device may comprise a variable-opening mechanical element such as a diaphragm, arranged upstream or downstream of the grating, so as to adjust the accessible diameter of the one or more openings. For example, the device comprises a diaphragm centered on the longitudinal axis of the transducer.

Some or all of the openings in the grating may be closed fully or partially in a controlled manner. This makes it possible, depending on the area to be treated, and in particular for the most sensitive areas such as those located in the vicinity of the eyes, to limit the surface area and the intensity of the treatment, if desired. The device may comprise a shutter able to move between a position of not blocking one or more openings in the grating and a blocking position in which the one or more openings are at least partially or even completely blocked. This shutter is for example controlled manually by the user as required.

The grating preferably has a thickness of between 0.1 and 10 mm, better still between 0.5 and 5 mm. This thus ensures a minimum distance of at least this thickness between the transducer and the keratin materials, regardless of the position of the grating with respect to the transducer.

The grating is preferably formed of a rigid or semi-rigid material, for example made of metal, or of rigid plastic. The grating is for example made of a material chosen from among a metal, in particular steel, stainless steel, an alloy, silicone, a polymer, in particular polyurethane (PU), polyethylene terephthalate (PET) or polythiophene (PT), or a combination of several of these elements.

The grating might not deform visibly to the naked eye during normal use due to its rigidity. As a variant, the grating may be deformable. It is possible for example to reduce its diameter by applying a mechanical pressure to its contour.

The grating may comprise recesses, in particular extending along its thickness. The grating comprises for example deformable channels, the diameter of which may vary according to the applied pressure, extending for example in a non-longitudinal direction. As a variant, the channels may be rigid in order to allow fluid to flow even if a pressure is applied to the grating.

The grating may also have reliefs or may or may not be flocked.

The grating may be formed with reliefs or be made of an intrinsically rough material, in order to have a non-smooth contact surface with the keratin materials. Such a surface state makes it possible to obtain a mechanical abrasive effect that may contribute to cleaning when the device is moved over the area to be cleaned during use.

The device may comprise a drive element for driving the grating, configured to generate an oscillating, vibrating and/or rotational movement of the grating on the keratin materials. For example, the device comprises a motor for driving the grating in movement, for example an alternating or non-alternating rotational, linear, eccentric movement or the like. The device may comprise a vibrator, where applicable, for vibrating the grating. The amplitude of the movement may be fixed or adjustable.

The mechanical abrasive effect may thus be obtained automatically due to the movement imposed by the device on the grating, and the impurities may be cleaned more effectively.

The grating may be produced in various ways.

The grating may comprise a peripheral frame, which defines the outer contour of the grating in a front-on view. This frame may be rigid, in particular more rigid than a central part of the grating, which is perforated.

The contour of the grating, in a front-on view, may have a circular, oval, polygonal shape or the like, and the grating preferably has a closed contour.

The one or more openings in the grating may have substantially the same size. The grating may comprise a plurality of openings arranged in a regular array, for example in rows or in columns.

As a variant, the one or more openings may be arranged concentrically.

The openings may have various shapes, in particular shapes of portions of one or more disks or rings.

The grating may have a single opening, in particular with a non-circular contour, for example in the shape of a slot, cross, regular or irregular polygon or oval. The grating preferably has at least two openings, in particular two openings having substantially the same size.

When a single opening is present, this may or may not be centered.

A grating having a fine mesh improves holding of the keratin surfaces to be treated during use of the device, thereby allowing better control of a constant distance between said materials and the emission surface for emitting acoustic waves, and thus of the cavitation phenomenon.

An opening in the grating may have an area of at least 1 mm². A smaller dimension of an opening in the grating, in a front-on view, is for example at least 0.5 mm.

The grating preferably has a ratio of total surface area of the openings with respect to the contact surface of the grating with the keratin materials greater than or equal to 0.3, better still 0.5, even better still 0.8.

Such a ratio makes it possible to avoid the keratin materials, in particular the skin, coming into contact with the transducer or deforming, while still keeping enough openings so that the ultrasonic waves reach the area to be treated or the vicinity thereof over an extent sufficient for the desired result.

Spacer with One or More Legs

Another subject of the invention, according to another of its aspects, is a device for cleaning human keratin materials (K) in contact with a cosmetic composition (C) within which bubbles of a gas are present and/or generated, the device comprising:
at least one ultrasonic transducer having an emission surface for emitting acoustic waves into the vicinity of said materials to be cleaned,
a spacer contributing to keeping the keratin materials at a predefined distance from the emission surface, this spacer advantageously comprising at least one leg extending axially over less than 360° about the axis of the transducer, in front of the emission surface.

“In front” should be understood to mean that the leg extends beyond the emission surface in the direction of the keratin materials to be treated.

The spacer may be in the form of a nozzle added to the rest of the treatment head by way of a fastening part, or be monolithic with the body thereof, for example.

The one or more legs may come to bear, by way of an end, directly against the keratin materials; this end may have a rounded head or a lug in order to be able to slide more easily on the skin, for example. As a variant, the one or more legs connect to a piece that extends for example transversely to the one or more legs, for example in the form of a ring or grating, and that comes to bear on the keratin materials.

The device may thus comprise a spacer without the abovementioned grating, a grating on its own without a spacer with one or more legs, or else a spacer having at least one leg connected to the abovementioned grating.

The grating may thus be carried by a spacer comprising a support part for the grating connected to at least one leg extending axially over less than 360° about the axis of the transducer, in front of the emission surface.

The one or more legs are preferably generally rectilinear, preferably parallel to the longitudinal axis of the transducer.

The one or more legs may extend toward the keratin materials in a direction substantially perpendicular to the emission surface for emitting ultrasonic waves.

As a variant, the one or more legs may extend so as to diverge moving away from the emission surface for emitting ultrasonic waves, thereby making it possible to improve the stability of the bearing of the device against the keratin materials.

The spacer may comprise a distal part intended to bear against the keratin materials, to which the one or more legs connect.

This distal part may extend at least partially facing the emission surface for emitting ultrasonic waves, or as a variant around same, when the device is observed along the longitudinal axis of the transducer.

This distal part has for example an annular shape, a bar shape or the shape of a sliding lug, being provided for example with at least one opening, and able to form the abovementioned grating.

The spacer is preferably laterally perforated. For example, the spacer comprises multiple legs, and the legs define perforations between them. For example, there are fewer than five support legs. The angular intervals between the legs, about the longitudinal axis of the transducer, may be greater than the angular extent of each leg, measured about said longitudinal axis.

The one or more legs may also themselves have one or more perforations.

Such perforations allow the composition to flow more easily in contact with the transducer during use of the device, and possibly allow the user to visually monitor the position of the transducer and the presence of composition in contact with the keratin materials more easily, the perforations forming windows allowing visual access to the space located in front of the transducer.

The perforations also facilitate the potential passage of a gas or of a light source, or temperature variations, caused in particular by a heat or cold production member of the device.

The fastening part of the nozzle that allows the one or more legs of the spacer to be fastened to the rest of the device, in particular to the treatment head, may be of any shape, in particular annular, and comprise fastening means for fastening by snap fitting, by screwing, by bayonet, by friction, magnetically, by clamping, in particular using at least one clamping screw or a hose clamp, or by any other appropriate means.

The position of the fastening part of the nozzle relative to the rest of the device is preferably adjustable and may be adjusted so as to adjust the distance at rest between the emission surface of the transducer and the keratin materials. The device may in particular be formed such that this position is adjustable by the user, for example by acting on an adjusting member such as a control knob or the like.

The spacer may have an adjustable height. For example, the nozzle may comprise multiple sub-parts able to be stacked according to the desired spacing.

The spacer may comprise an elastic return member, preferably at least one helical spring or leaf spring, designed to elastically urge the distal end of the spacer away from the emission surface for emitting acoustic waves and to make it possible to control the application pressure of the spacer to the keratin materials, thereby allowing greater comfort when using the device.

The one or more legs are for example formed telescopically, and the elastic return member acts so as to urge them to deploy.

The legs may also be received in a sliding manner, on the side of their proximal end, in a guide, and the elastic return member opposes their insertion into this guide.

The grating, when fastened to the spacer, may thus be mobile relative to the rest of the device, and returned by an elastic return means to an initial position, the elastic return means acting so as to apply the grating against the keratin materials to be treated.

Where applicable, the return movement of the grating or of a mobile part of the spacer, following the application of the device against the keratin materials to be treated, is limited by a stop against which the grating or the mobile part comes to bear. The return movement of the grating or of this mobile part may also be detected in order to automatically trigger the emission of the acoustic waves when the device is applied against the keratin materials.

The grating and its support part that holds it in place relative to the emission surface may or may not be formed of different materials. Where applicable, the support part and the grating are in a single piece, and for example are produced monolithically by injection molding, machining or 3D printing.

The one or more legs and/or the grating may also conduct current, in order for example to deliver an electric current into the composition and/or to the materials to be treated.

Flow of the Composition in the Spacer and/or the Grating

The device according to the invention may comprise at least one supply duct opening out close to the keratin materials to be treated and making it possible to distribute the composition in contact therewith, in particular at least one duct extending along the one or more legs, or therein, or more generally in the support part for the grating and/or in said grating.

When the spacer carries a grating at its end in contact with the keratin materials, the supply duct may extend in the grating or the support part for the grating.

Such a device makes it possible to apply the cosmetic composition continuously, that is to say that a flow of the composition is established in contact with the keratin materials to be treated, this flow taking place for example in a closed or open circuit.

When the flow is established in a closed circuit, the device may comprise at least one return duct connected to a suctioning pump, making it possible to at least partially recover the composition in the vicinity of the keratin materials to be cleaned, in particular at least one duct extending along the one or more legs, and possibly in the grating or the support part for the grating.

Method for Cleaning Keratin Materials with a Grating and/or Spacer

Another subject of the invention is a method for cleaning human keratin materials in contact with a cosmetic composition within which bubbles of a gas are present and/or generated, this method comprising the steps of:
Putting the grating or the spacer of the device in contact with said keratin materials to be cleaned,
Subjecting bubbles of the gas to acoustic waves emitted by the ultrasonic transducer into the vicinity of said materials to be cleaned, in order in particular to cause them to burst and generate a mechanical shock on the surface to be cleaned in order to remove soiling therefrom.

The acoustic waves may propagate through at least one or two openings in the grating.

The grating or the spacer may be moved during the treatment, for example intermittently, by remaining stationary for a certain duration, and then moved incrementally along the surface to be treated. The grating or the spacer may also be moved continuously over the surface to be treated.

As mentioned above, the grating or the spacer may be driven in a movement, in particular oscillating movement, that is combined with the movement imparted by the user.

The grating or the spacer, by being moved in contact with the keratin materials to be treated, may bring about abrasion thereof.

### Flexible Lip

The treatment head may comprise at least one flexible lip, which may also act as a spacer so as to maintain the abovementioned spacing between the emission surface for emitting acoustic waves of the transducer and the surface to be treated.

The flexible lip, when the treatment head is applied to the keratin materials to be treated, may contribute to confining the fluid in the treatment area.

The lip may consist of sub-lips between which the fluid may be recovered.

The flexible lip is preferably formed with a material comprising silicone, polyurethane (PU), a natural or synthetic rubber, plastic, polyethylene terephthalate (PET) or else polythiophene (PT), or a combination of several of these elements.

The flexible lip may be formed with reliefs or be made of an intrinsically rough material, in order to have a non-smooth contact surface with the keratin materials. The flexible lip may thus contribute to cleaning the keratin materials through a mechanical abrasive effect obtained when moving the device over the area to be cleaned during use.

This flexible lip may also contribute to delivering, spreading, collecting and/or recycling the fluid.

### Battery

The device preferably comprises at least one battery, in particular a removable battery. The battery may serve to power the whole device, or just part, in particular the pump.

The battery may be of various types, for example Li-ion or NiMH, preferably of a type suited to wet conditions or that is able to be at least partially submerged in a liquid.

The battery is preferably rechargeable, in particular in a wired manner or by induction, or when the device is placed on a support and/or a docking station for recharging the battery.

The battery is preferably selected so as to ensure, without having to be recharged, that the device is powered for multiple consecutive treatments, or even multiple treatments and cleaning of the device.

### Other Elements

The device may comprise an electronic circuit driving the operation of the transducer and possibly other elements of the device, such as the electric pump.

The electronic circuit preferably comprises a control unit, for example with a microcontroller, which may be carried by the handpiece and/or distributed between a potential base station and the handpiece, or present just on the base station.

The device may comprise multiple sensors other than those described above, such as a sensor for sensing contact of the treatment head on the keratin materials to be treated, or a tank filling sensor, which are able to communicate data to the control unit.

The device may also comprise a human-machine interface communicating with the control unit. The human-machine interface may comprise a display screen and/or control buttons.

The interface may offer various functions and/or programs for example for adjusting certain operating parameters of the device or for indicating certain information to the user, such as battery level, usage time, liquid level in the tank, clogging level of the various washable elements (tank, filters, etc.), information specific to the user, or even information relating to safety and/or to the usage conditions of the device.

The interface may also communicate, via a wired or wireless link, for example via Wi-Fi or Bluetooth, with a terminal such as a mobile telephone, in order for example to receive updates or to send data linked to the use of the device.

The interface may also comprise functionalities for diagnosing the keratin materials to be treated.

The device may comprise various switches, in particular an on switch and/or standby switch, a program selection switch or a switch for selecting certain parameters specific to the ultrasonic waves, or else for selecting the flow rate of the pump.

The device may also comprise one or more LED light sources able to serve to illuminate the area to be treated or else act as indicators, notifying for example the user of the need to change, fill or empty the tank, or to clean the filtration system.

As described above, the device may comprise a base station connected to the handpiece, which may be a station for recharging and/or storing the handpiece.

### Bubble Generator

Preferably, as described above, the bubbles are generated only by the acoustic waves in the cavitation phenomenon, obtained through a minimum level of the parameter Isata and through the other abovementioned parameters.

The device thus preferably does not comprise a bubble generator.

As a variant, the device may comprise a bubble generator for generating additional bubbles within the fluid.

The bubble generator may be arranged in the device so as to generate bubbles prior to, at the same time as or cyclically with respect to the emission of the acoustic waves.

The bubble generator may employ any technique suitable for generating bubbles, for example using the abovementioned mechanical, physical, chemical or electrochemical means.

The bubble generator may thus employ the techniques of decompressing liquids, creating a turbulent flow or supplying energy, as described above.

### Method for Treating the Hair

Another subject of the invention is a method for treating the human hair, in particular for cleaning and/or bleaching the hair, in contact with a fluid, in particular a cosmetic composition, within which bubbles of a gas are present and/or generated, the method comprising the step of:

subjecting the bubbles of gas to acoustic waves emitted by a transducer into the vicinity of hair to be treated, in order to cause said bubbles to burst and generate a mechanical shock on the surface of the hair to be treated, in particular in order to remove impurities and/or dyes therefrom.

The transducer may in particular belong to a treatment device according to the invention, as defined above, comprising a guiding and/or combing member for guiding and/or combing the hair.

The method may thus comprise guiding and/or combing the hair before and/or after it passes through a treatment space, and/or in the treatment space itself.

The method may comprise guiding the hair, aimed at bringing said hair into or keeping said hair in the treatment space, and/or at keeping said hair, when it passes through the treatment space, at a predefined distance from the emission surface for emitting acoustic waves, in particular preventing said hair from moving away by more than a given distance from the emission surface for emitting acoustic waves.

The method may comprise vibrating and/or rotating and/or longitudinally and/or transversely moving the guiding and/or combing member and/or the transducer.

The method according to the invention may comprise the step of the user adjusting the distance between a guiding surface of the guiding and/or combing member and the emission surface of the transducer in order to adapt the method to the desired treatment and/or to the treated hair.

The method may comprise the step of the user adjusting the internal cross section, in particular the diameter, of a cavity defined by the guiding and/or combing member in order to adapt the method to the treated hair and to the desired treatment.

The method may comprise the step of the user adjusting the distance between a proximal part, closer to the transducer, of the guiding and/or combing member and a distal part thereof that is further away. This adjustment may make it possible to adapt the method to the hair and/or to the desired treatment.

The method according to the invention may comprise the device heating and/or the device diffusing light in order for example to activate certain compounds present in the fluid, in particular in the cosmetic composition.

The method for treating the hair may comprise a step of applying the fluid, in particular the cosmetic composition, to the strand of hair.

The fluid, in particular the cosmetic composition, may be supplied to the treatment space by the guiding and/or combing member, in particular when this is hollow or when this is passed through by a duct. This application may take place continuously or discontinuously. The fluid, in particular the cosmetic composition, may be delivered to the hair via one or more orifices of the guiding and/or combing member. As a variant, the fluid, in particular the cosmetic composition, is delivered in another way, for example by another application element of the device, or applied to the hair in a way other than with the device.

Generally speaking, the fluid, in particular the cosmetic composition, may in itself, due to its formulation, already contribute to the bleaching and/or to the removal of impurities or dyes that it is desired to remove through the action of the bubbles subjected to the acoustic waves. The action of the bubbles subjected to the acoustic waves may speed up or improve this process. The association of the wave generated by the bursting of the bubbles in combination with the action of the fluid, in particular the cosmetic composition, may thus, by synergy, have an effect greater than that of just the waves or than that of just the fluid, in particular the cosmetic composition.

The bubbles are preferably generated within the fluid, in particular the cosmetic composition, by the acoustic waves, which may then cause them to burst. The method may thus comprise applying a bubble-free fluid, the bubbles forming under the action of the acoustic waves. This makes it possible to avoid using a bubble generator and simplifies the implementation of the device.

As a variant, or in addition, the fluid, in particular the cosmetic composition, is applied first of all, with bubbles already present within it or not, and then exposed, second of all, after it has been applied to the strand of hair, to the acoustic waves in order to bring about shockwaves following the bursting of the bubbles. The bubbles may be formed using a bubble generator of the device, operating for example by electrolysis.

For example, the user first applies a cosmetic composition in the form of foam to the area to be treated, for example by spraying it onto said area, in particular along the strand, and then brings a treatment device into contact with the composition, in order to subject it to the acoustic waves.

The fluid, in particular the cosmetic composition, may be applied continuously, that is to say that a flow of the fluid, in particular the cosmetic composition, is established in contact with the hair to be treated, this flow being for example in a closed or open circuit.

When the flow takes place in a closed circuit, the fluid, in particular the cosmetic composition, may be recycled at least partially. An additional amount of fluid, in particular cosmetic composition, may be introduced continuously or intermittently into the circuit in order to mitigate losses.

In an open circuit, the fluid, in particular the cosmetic composition, is not recycled to implement the method, and is for example siphoned off to a collection container or directly with wastewater.

The flow of the fluid, in particular the cosmetic composition, takes place for example at a flow rate of between 0.01 mL per second and 50 mL per second.

The method may comprise dissolving a sleeve made of fabric or a nonwoven sleeve, in particular made of polymer, covering part of the guiding and/or combing member. This dissolution may take place as the fluid exits, for example via one or more orifices located on the guiding and/or combing member.

The method according to the invention may be applied to hair that has undergone a dyeing treatment, for example what is known as permanent dyeing, what is known as semi-permanent dyeing, what is known as tone on tone dyeing, strand dyeing, what is known as swept dyeing, henna dyeing, or else through the use of a tinted shampoo.

The method according to the invention may be implemented in order to bleach the hair, without being followed by new dyeing within 24 hours. The method may also be implemented, and then followed within 24 hours by a new treatment of dyeing the hair bleached by the ultrasonic treatment according to the invention.

The method according to the invention may also be implemented to create a dyeing gradient and/or at least one pattern along the strand by varying for example the intensity of the treatment and/or the duration depending on the position along the strand.

The method may be implemented to bleach the hair only on the outside of the hair (that is to say on the side opposite the scalp), so as to give the hair a swept effect.

The method according to the invention, when used on hair to bleach the hair in conjunction with a chemical bleaching means, may make it possible to reduce the amount of active bleaching agents and/or the exposure time to these active agents.

The method may thus comprise exposing the hair both to the acoustic waves according to the invention and to at least one active bleaching agent, for example a persulfate, an alkaline or an oxidizing agent, such as hydrogen peroxide, potassium salt, sodium salt, or ammonium perborate or percarbonate salt.

The risks of irritation linked to the use of active bleaching agents may be reduced, or even eliminated.

The method may comprise selectively treating part of the hair, by generating the emission of acoustic waves only in certain areas of the hair, for example by making the device operate intermittently.

Another subject of the invention, independently or in combination with the above, is a method for treating at least one strand of hair that has undergone a permanent or semi-permanent dyeing treatment, in which said strand, in contact with a fluid, such as a cosmetic composition containing at least one surfactant, is exposed to acoustic waves generated by a transducer, of a frequency and intensity chosen so as to generate, in the composition, bubbles through cavitation and to cause them to burst, the bubbles present in the treatment area being or having been mostly generated by the transducer.

“Permanent dyeing”, or oxidization dyeing, denotes a dye that covers all of the hair while penetrating the core of the hair and which, as its name indicates, is designed to last.

“Semi-permanent dyeing” denotes a type of dyeing, in particular without ammonia, that covers the surface without reaching the core of the hair, and which gradually washes out over the course of being shampooed.

The bleaching method according to the invention may comprise, following exposure to the acoustic waves, drying the hair using a hairdryer.

The bleaching method may be followed by dyeing, for example permanent or semi-permanent dyeing, with the same color as that used to color the hair that was treated in order to bleach said hair, or with a different color.

Throughout the following description, the invention is implemented with a fluid that is an aqueous medium. However, it may be implemented more generally with any fluid compatible with domestic use by a consumer or in a hair salon.

### BRIEF DESCRIPTION OF THE DRAWINGS

The invention will be able to be better understood upon reading the following detailed description of non-limiting exemplary implementations thereof, and upon examining the appended drawing, in which:

FIG. 1 schematically illustrates the use of one example of a treatment device according to the invention,

FIG. 2 is a schematic longitudinal sectional view of another example of a treatment device for implementing the invention,

FIG. 3 is a view, similar to FIG. 2, of one variant implementation of the invention,

FIG. 4 schematically and partially shows one variant embodiment of the device according to the invention,

FIG. 5 is a perspective view of the variant of FIG. 4,

FIG. 6a and FIG. 6b show details of the example of FIG. 4,

FIG. 7 schematically and partially shows one variant of a fluidic circuit according to the invention,

FIG. 8 is a block diagram illustrating one example of operation of the device of the invention,

FIG. 9 schematically and partially shows another variant of a fluidic circuit according to the invention,

FIG. 10 schematically and partially shows one variant embodiment,

FIG. 11a to FIG. 11e schematically and partially illustrate variants of transducers according to the invention,

FIG. 12a and FIG. 12b schematically and partially show examples of transducers having reliefs,

FIG. 13a to FIG. 13c schematically and partially show examples of such reliefs,

FIG. 14 schematically and partially shows one example of a vibrator according to the invention,

FIG. 15 partially and schematically illustrates another example of a treatment device according to the invention,

FIG. 16 partially and schematically shows the possibility of adjusting the resting distance between the transducer and the grating of the device of FIG. 1,

FIG. 17a, FIG. 17b, FIG. 17c, FIG. 17d, FIG. 17e and FIG. 17f partially and schematically show variant embodiments of the grating in examples of devices according to the invention,

FIG. 18a to FIG. 18e schematically and partially show spacer implementation variants,

FIG. 19a and FIG. 19b schematically and partially show other variant embodiments of the device according to the invention,

FIG. 20 schematically and partially shows another variant embodiment of the device according to the invention,

FIG. 21 partially and schematically shows one example of a modulated electrical signal exciting the one or more ultrasonic transducers,

FIG. 22 is a partial and schematic depiction of one example of an electrical circuit comprising the transducer,

FIG. 23 partially and schematically shows one example of a treatment device according to the invention,

FIG. 24 is a view, similar to FIG. 1, of one variant of a treatment device according to the invention,

FIG. 25 is a view, similar to FIG. 1, of one variant of a treatment device according to the invention,

FIG. 26 is a view, similar to FIG. 1, of one variant of a treatment device according to the invention,

FIG. 27 is a view, similar to FIG. 1, of one variant of a treatment device according to the invention,

FIG. 28 is a view, similar to FIG. 1, of one variant of a treatment device according to the invention,

FIG. 29 is a view, similar to FIG. 1, of one variant of a treatment device according to the invention,

FIG. 30 is a view, similar to FIG. 1, of one variant of a treatment device according to the invention,

FIG. 31 is a view, similar to FIG. 1, of one variant of a treatment device according to the invention,

FIG. 32 is a view, similar to FIG. 1, of one variant of a treatment device according to the invention,

FIG. 33 is a view, similar to FIG. 1, of one variant of a treatment device according to the invention,

FIG. 34 is a view, similar to FIG. 1, of one variant of a treatment device according to the invention,

FIG. 35 shows the color intensity within the hair fibers for three hair samples,

FIG. 36 shows the fluorescein staining level of the hair for the same three hair samples as in FIG. 14,

FIG. 37 shows dyed and untreated strands of hair, A and B, and dyed and treated strands of hair, C and D,

FIG. 38 shows the color intensity in the hair for five hair samples,

FIG. 39 shows the results of statistical analysis of the color intensity for the same five hair samples as in FIG. 16,

FIG. 40a, FIG. 40b, FIG. 40c, FIG. 40d, FIG. 40e, FIG. 41a, FIG. 41b, FIG. 41c, FIG. 41d, FIG. 41e, FIG. 42a, FIG. 42b, FIG. 42c, FIG. 42d, FIG. 42e, FIG. 43a, FIG. 43b, FIG. 43c, FIG. 43d, FIG. 43e, FIG. 44a, FIG. 44b, FIG. 44c, FIG. 44d, FIG. 44e, FIG. 45a, FIG. 45b, FIG. 45c and FIG. 45d show computed response surfaces illustrating examples of parameters giving satisfactory cleaning effectiveness according to the method of the invention, and

FIG. 45e shows an empty response surface illustrating one example of a combination of parameters not giving the desired cleaning effectiveness.

### DETAILED DESCRIPTION

The method according to the invention comprises generating bubbles within a cosmetic composition and bursting them through exposure to acoustic waves.

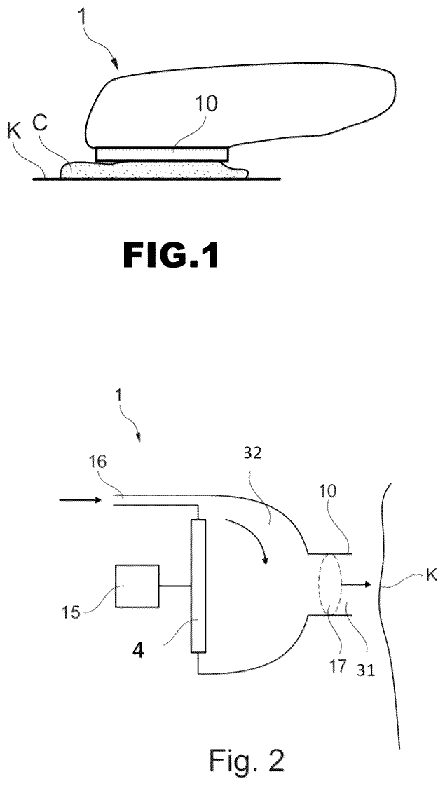

FIG. 1 illustrates a first exemplary implementation of the invention, in which a cosmetic composition C is present on the surface of the keratin materials K to be treated, and a treatment device 1 is brought into contact with the composition C in order to emit acoustic waves therein.

The composition C might not comprise bubbles before the acoustic waves are emitted. As a variant, the composition C already comprises bubbles, for example in the form of foam, and bubbles are also formed by the acoustic waves.

The keratin materials K are formed for example by the facial skin or the hair.

It is desired for example to clean the skin in order to remove traces of make-up more quickly and effectively.

The treatment device 1 comprises a handpiece that carries a sonotrode 4, in contact with the composition, and from which the acoustic waves are emitted.

The handpiece may be manipulated so as to form a slight space with the keratin materials K and avoid contact between the sonotrode 4 and said keratin materials.

As a variant, the handpiece is designed to maintain such a spacing, by virtue of one or more elements 49 intended to come into contact with the keratin materials and from which the sonotrode 4 is set back.

Under the effect of the acoustic waves, bubbles are generated in the composition C and then burst on themselves, which generates a shockwave, which proves to be effective for cleaning the skin.

In the example of FIG. 1, the composition C is for example applied from a pressurized container, which generates a foam, and then the handpiece carrying the sonotrode 4 is brought into contact therewith.

The composition may also be applied, as illustrated in FIG. 2, by the device 1 that generates the acoustic waves.

In this figure, the device 1 comprises a treatment head 10 designed to distribute the composition C over the area to be treated, for example via at least one opening 31.

The device 1 may comprise, as illustrated, a chamber 32 in which the composition C flows and at least one ultrasonic transducer 4 for emitting acoustic waves into the chamber 32. The transducer 4 is powered by a generator 15, which may or may not form part of the handpiece, being for example present within a base station to which the handpiece is connected by a cable.

The composition C may be supplied to the chamber 32 by a duct 16, and come for example from a composition tank 22.

In the example of FIG. 2, the handpiece is moved along the area to be treated and the composition that is delivered via the opening 31 is not recycled.

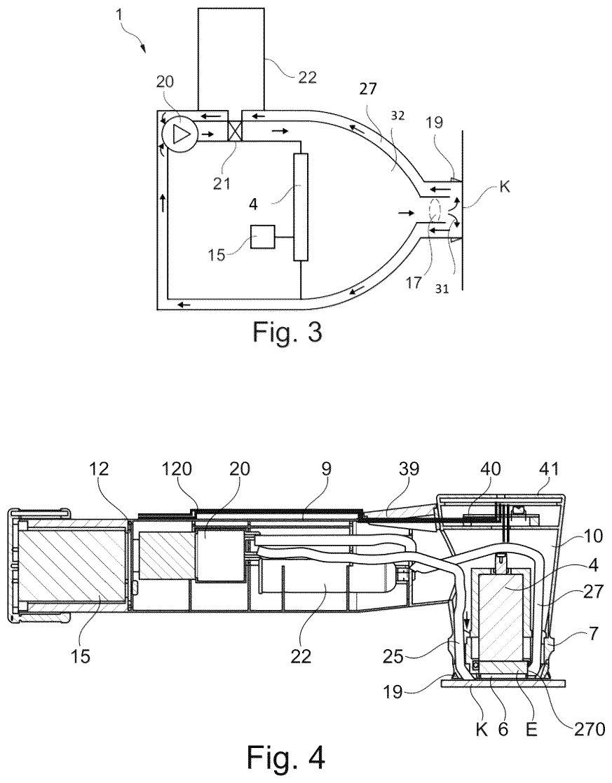

In the variant of FIG. 3, the composition is recycled.

In this example of FIG. 3, the device 1 used to implement the method according to the invention comprises at least one transducer 4 emitting acoustic waves into a chamber 32, as in the example of FIG. 2.

However, the composition C that is distributed via the opening 31 onto the area to be treated K is recovered via at least one duct 27 with a view to being recycled.

In the example under consideration, this duct 27 opens out around the opening 31 so as to recover the composition that has been in contact with the area to be treated.

The device 1 may comprise, where applicable, around the duct 27, a sealing element 19, such as a flexible lip, for containing the composition and facilitating its return via the duct 27. The lip may consist of sub-lips between which the liquid may be recovered.

The duct 27 communicates with a suctioning pump 20, for example an electric pump, which may, as illustrated, send the returned composition to a filter 21. Said filter may be designed to stop for example particles suspended in the composition, such as skin debris removed during the cleaning, for example.

The composition is returned to the chamber at the outlet of the filter 21.

The composition may come from a tank 22 that is shown schematically, for example carried by the handpiece.

This tank 22 makes it possible to fill the circuit in which the composition flows during operation of the device, and to compensate for any losses in composition in the event of some of said composition not being recycled.

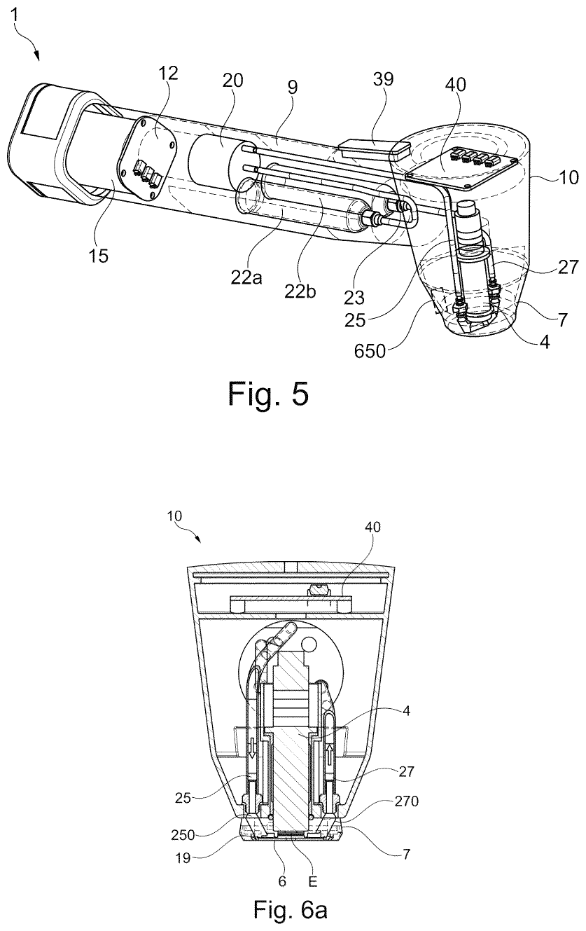

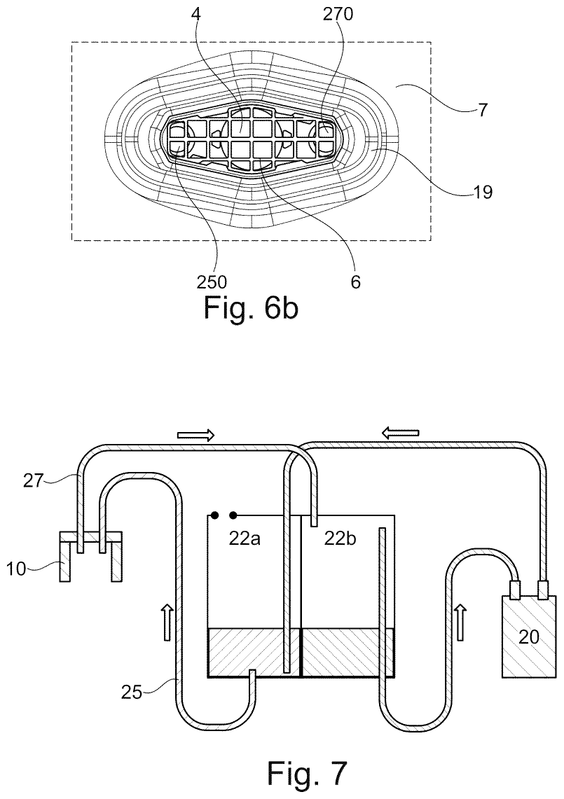

FIGS. 4 and 5, and corresponding detailed views in FIGS. 6a and 6b, show another example of a device according to the invention, comprising an ultrasonic transducer 4, two tanks 22a and 22b, a pump 20 and a fluidic circuit comprising ducts 23, 25 and 27 allowing the composition C to flow between the tanks 22a and 22b and the keratin materials K to be treated.

In the example under consideration, the device 1 is in the form of a handpiece comprising a handle 9 and a treatment head 10 comprising a spacer 7 in contact with the keratin materials K to be treated.

The device does not comprise a bubble generator other than the transducer 4, the bubbles being generated in the cavitation phenomenon by virtue of the acoustic waves.

The transducer 4 is arranged relative to the spacer 7 so as to form a space E between the emission surface for emitting acoustic waves and the surface of the keratin materials K to be treated.

The cavitation of the bubbles takes place in this space E, as illustrated in FIG. 6a.

The device 1 may comprise a grating 6 in contact with the keratin materials to be treated, for example fastened to the spacer 7, comprising for example regular openings arranged in rows and in columns, as shown in FIG. 6b.

The device 1 furthermore comprises an electronic circuit 12 connected, by a connector 120, to an electronic generator 40 powering the transducer, as illustrated in FIG. 4. This generator comprises for example an oscillator and a power stage.

The electronic circuit 12 may comprise a control unit able to communicate with a human-machine interface 41, which may comprise a screen, in particular an LED screen and/or control buttons, or even communicate via a wireless link with a terminal such as a mobile telephone.

The human-machine interface 41 makes it possible to adjust certain operating parameters of the device, for example the intensity with which the acoustic waves are emitted.

The electronic circuit 12 is able to drive the operation of the pump 20, of a battery 15 powering the device (in particular its load) and receive data from one or more sensors (not shown) such as a sensor for sensing contact of the device on the skin.

In the latter case, the electronic circuit 12 may start the pump 20 and emit acoustic waves only when the area to be treated is in contact with the grating 6, in particular in a way that allows the composition to be recycled.

The electronic circuit may also communicate with an optical detector 650, as shown highly schematically in FIG. 5, in order to detect the presence of a compound to be removed from the surface of the skin, and possibly determine the amount that is present.

As mentioned above, the system for the flow of the composition, comprising the fluidic circuit, the one or more tanks and the pump, may be arranged in various ways.

In the example shown in FIGS. 4 and 5, the device 1 comprises two separate tanks 22a and 22b. The tank 22a is for example a tank for composition to be distributed; it is connected to the pump 20, which may, as illustrated, send the composition C to the treatment area via a duct 25 opening out via an outlet 250.

The tank 22b recovers the composition that has been in contact with the area to be treated by way of a duct 27 that opens out onto an outlet 270, as illustrated in FIG. 6a.

The device 1 may comprise, where applicable, on the perimeter of the end of the treatment head, a sealing element 19, such as a flexible lip, for containing the composition and facilitating its return via the duct 27.

The tanks 22a and 22b may be at least partially transparent, thereby allowing the user to visually monitor the fill level and/or clogging level thereof, through where applicable a transparent window provided in the handle. Each of the tanks and/or the assembly formed by the one or more tanks and the fluidic circuit is preferably removable in order to be able to be cleaned or replaced easily by the user.

A filter may also be arranged in a duct connecting the tanks 22a and 22b.

The filter may also be carried by the tank 22b, for example so as to allow it to be replaced automatically when the composition is drained and the tank is replaced.

In the variant illustrated in FIG. 7, the tanks 22a and 22b are connected directly to the treatment area via ducts 25 and 27, respectively.

In this example, the pump 20 is connected to the tanks 22a and 22b without otherwise having composition flowing within it; the pump makes it possible to generate a positive air pressure in the tank 22a for composition to be distributed, and a negative pressure in the tank 22b for recovered composition, in order to maintain the flow in the ducts 25 and 27 between the treatment area and the tanks.

The pump 20 may also contribute to generating bubbles in the tank 22a, prior to the distribution of composition over the treatment area.

In one variant, the device may also comprise a single tank 22, which may or may not comprise an internal filter, this possibly being removable.

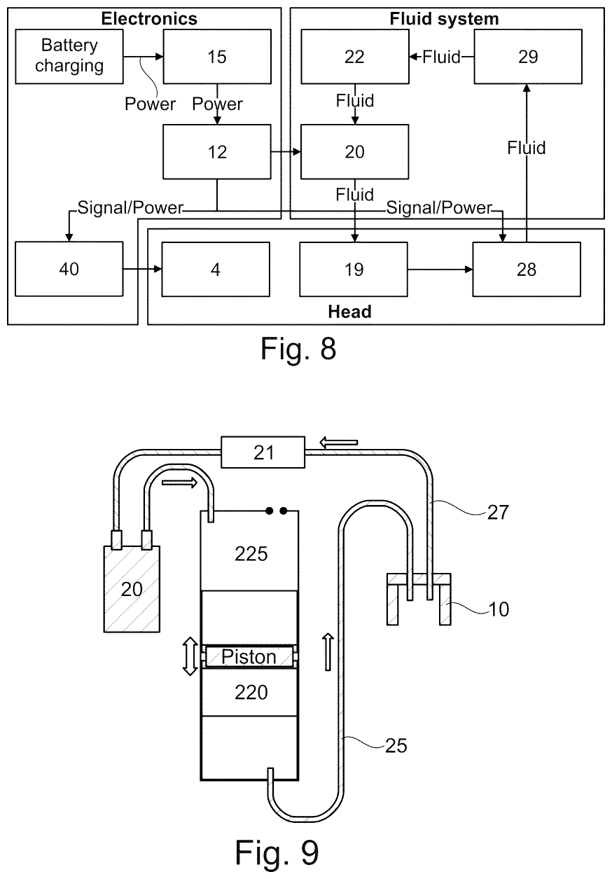

For example, the composition is recovered using a suction system 28 such as a suctioning pump, which may be arranged at various locations of the fluidic circuit, for example directly in the treatment head 7 where the composition is collected and kept by virtue of the flexible lip 19, as illustrated schematically in FIG. 8.

The device 1 may comprise, as illustrated in FIG. 8, a treatment and purification unit 29 for the composition, which allows the composition to be recycled with a view to being returned to the keratin materials to be treated, the composition then flowing at least partially in a closed circuit.

The treatment and purification unit 29 is for example a filtration system, shown schematically in FIG. 8, comprising one or more filters designed to stop particles suspended in the composition, such as skin debris removed during cleaning.

The filtration system may be arranged between the suction system 28 and the tank 22, as illustrated in FIG. 8.

In the variant illustrated in FIG. 9, the tank 22 comprises two compartments 220 and 225 separated by a piston 230. The compartment 220 contains for example the composition C to be distributed and is connected directly to the treatment head by a duct 25 that opens out on the treatment area.

The composition is recovered via a duct 27 that connects the treatment area to a pump 20, this returning the used composition to the second compartment 225 of the tank 22.

A filter 21 may be arranged in the duct 27 between the treatment area and the pump 20, as illustrated.

The flow is established by virtue of the pump 20 and the movement of the piston 230 within the tank 22. In this example, the compartments 220 and 225 of the tank 22 do not communicate.

The tank 22 is preferably partly or fully transparent so that the user is able to assess the position of the piston and the respective remaining levels of composition to be distributed and used liquid.

The device may furthermore comprise one or more control buttons 39, for example an on button or standby button, as illustrated in FIGS. 4 and 5.

The device is for example charged using a USB port (not shown) positioned directly on the body of the apparatus, or else by being placed on a base station provided for this purpose, and be charged inductively, or using any appropriate connector.

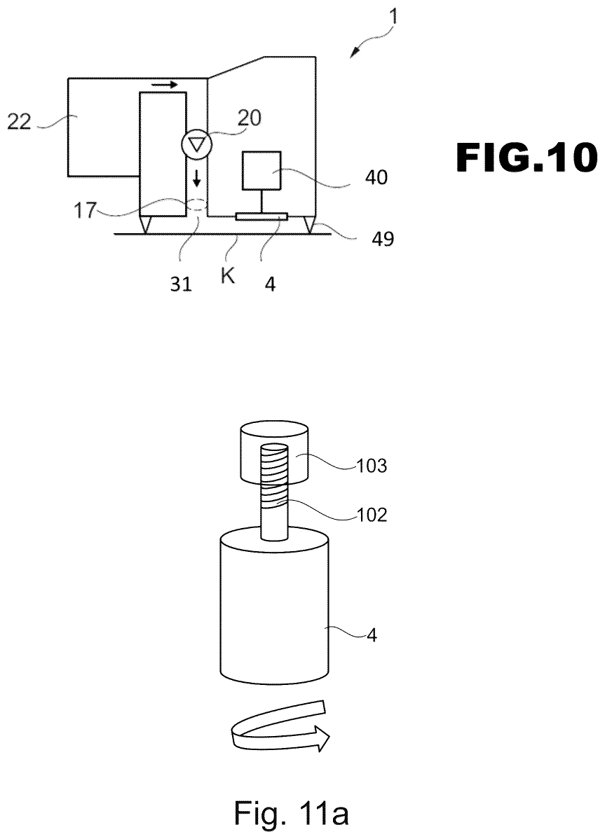

The battery 15 may also be removed and charged separately. The device may also comprise, as illustrated in FIG. 10, an outlet 31 for delivering the composition C and a transducer 4 arranged in a manner offset from the outlet 31. In this case, the composition deposited on the area to be treated K, after the device has been moved relative to the area to be treated, passes under the transducer 4, where it is exposed to the acoustic waves.

The device may comprise a possible additional bubble generator 17.

A spacing element 49, which may be a flexible lip, may serve to space the transducer 4 from the area to be treated in order to avoid direct contact with the skin, for example.

The transducer 4 or its sonotrode are preferably removable. It is thus possible to easily remove them from the device in order to clean them.

It is also possible to interchange various transducers and sonotrodes, in particular to provide sonotrodes of different sizes and/or transducers generating waves of varied frequency and acoustic intensity, depending on the desired use of the device.

To this end, the transducer or the sonotrode may comprise various fastening means for fastening to the handpiece.

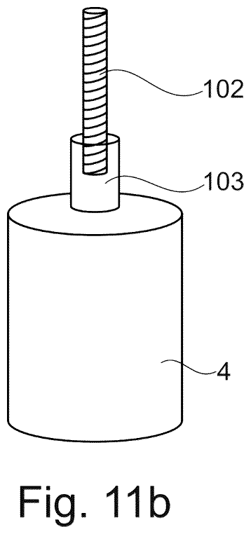

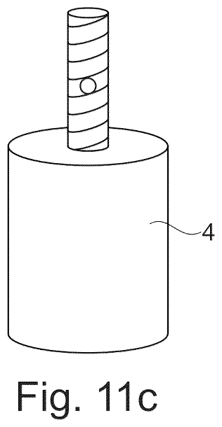

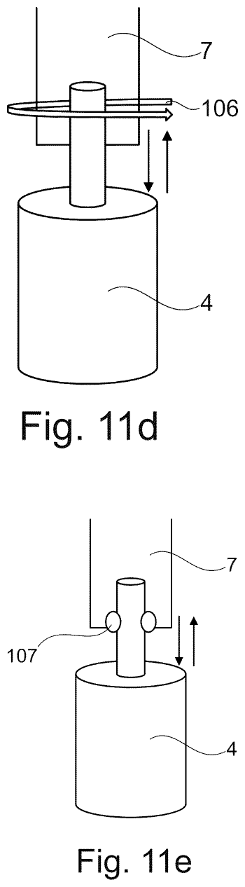

The fastening means are for example screwed fastening means, as illustrated in FIGS. 11a and 11b.

The transducer 4 may have a threaded rod 102 at one of its ends, as illustrated in FIG. 11a, which is able to be screwed into a nut 103 fastened to the device, in particular into the treatment head.

By contrast, the transducer 4 may comprise a nut 103 on its upper face and the threaded rod 102 may be fastened to the treatment head 7, as shown in FIG. 11b.

The removable transducer may also be fastened by snap fitting, as illustrated in FIG. 11c, or by clamping, in particular by way of hose clamps 106, as illustrated in FIG. 11d, or a seal 107, for example made of polymer or rubber, as illustrated in FIG. 11e.

The emission surface for emitting acoustic waves may have reliefs 420 where the bubbles may arise during cavitation, at various levels along the longitudinal axis X of the transducer.

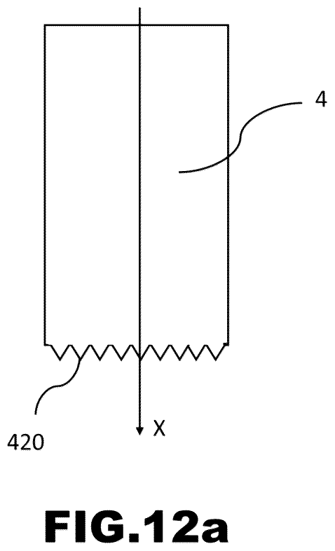

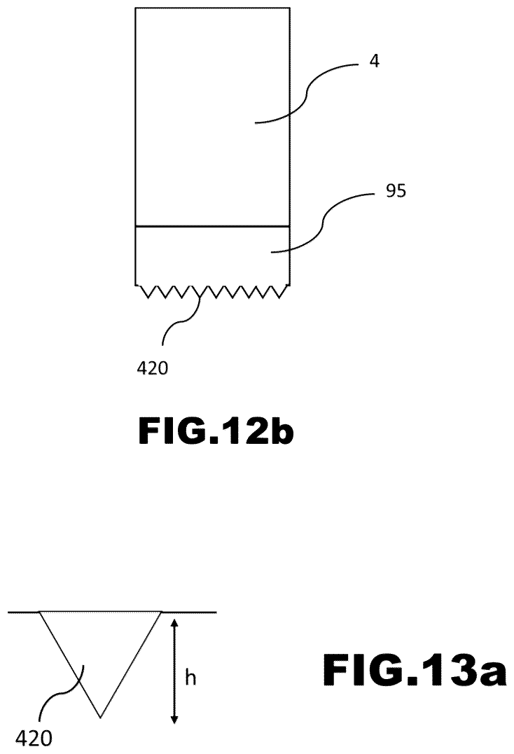

These reliefs 420 may be monolithic with the sonotrode, as illustrated in FIG. 12a, or may be formed on an added part 95 added to the sonotrode, as illustrated in FIG. 12b, which is for example fastened to the sonotrode by screwing.

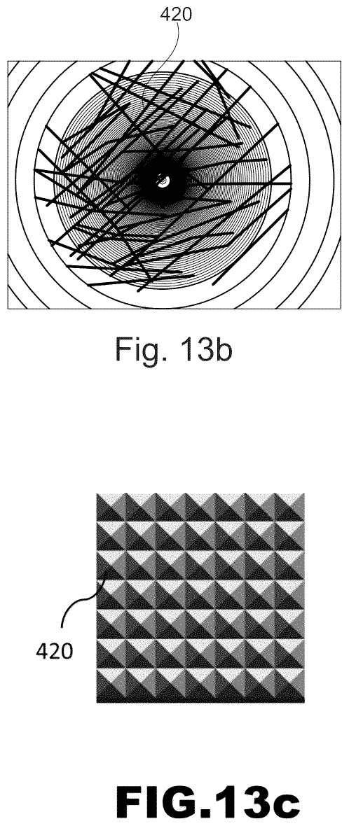

All or some of the reliefs 420 may have a random distribution over the emission surface and/or a random size, as illustrated in FIG. 13a.

As a variant, the reliefs may have a predefined shape, for example a pyramidal shape, as illustrated in FIG. 13b, and be arranged in a regular arrangement, for example in a regular array, as illustrated in FIG. 13c.

The reliefs 420 preferably have a height h, measured parallel to the longitudinal axis X of the sonotrode, of between 0.001 mm and 50 mm, better still 0.01 mm and 30 mm, better still between 0.1 mm and 1 mm.

The device may also comprise a vibrator 750, making it possible to subject at least part of the emission surface to additional vibrations of a frequency lower than that of the acoustic waves.

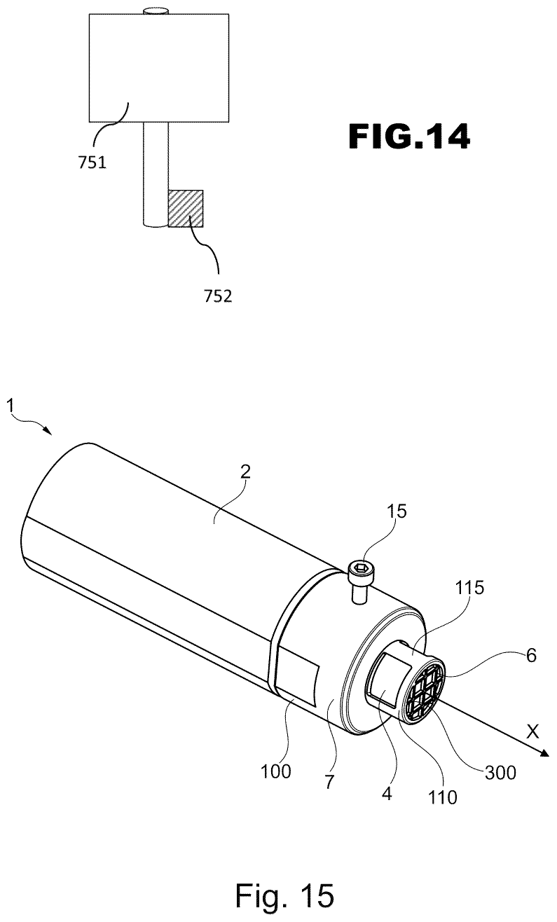

The vibrator 750 comprises for example a motor 751 driving an imbalance 752 in rotation, as illustrated in FIG. 14.

FIG. 15 shows another example of a device 1 according to the invention. The device 1 comprises a body 2 housing a transducer 4, a spacer 7 in the form of a nozzle added to the treatment head, and a grating 6 arranged in front of the transducer 4.

The transducer 4 comprises electroactive elements coupled to a sonotrode, typically made of metal, which defines an emission surface S via which the acoustic waves are emitted to the keratin materials to be treated.

The body 2 of the treatment device 1 may be in the form of a handpiece that is manipulated by the user so as to bring the grating 6 into contact with the area to be treated.

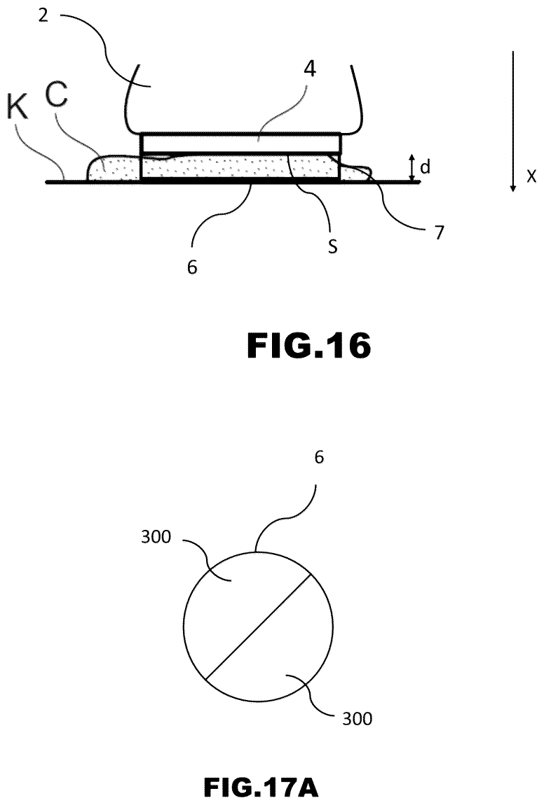

As illustrated in FIG. 16, a composition C containing bubbles B is present during use of the device 1 on the surface of the keratin materials K to be cleaned.

The composition C is for example in the form of foam or of another composition, applied to the keratin materials before bringing the device into contact therewith. The composition C may also be delivered by the device 1, or be partially present before the device 1 is brought into contact with the keratin materials and partially supplied by the device.

The keratin materials K are formed for example by the facial skin or by the hair.

The transducer 4 is in contact with the composition C.

Under the effect of the acoustic waves, the bubbles burst on themselves, which generates a shockwave, which proves to be effective for cleaning the keratin materials K. The acoustic waves may also contribute to forming the bubbles, where applicable.

The grating 6 may be produced in various ways.

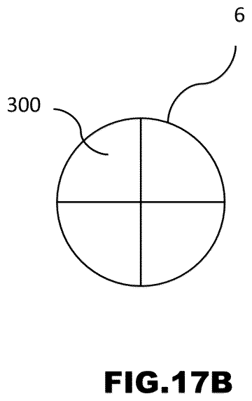

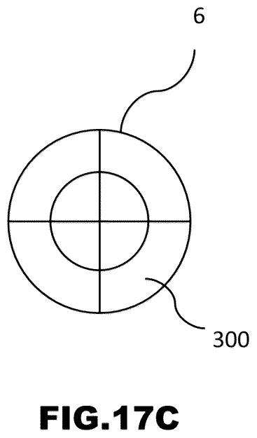

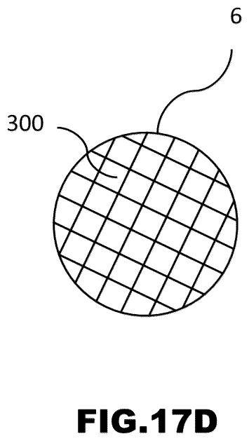

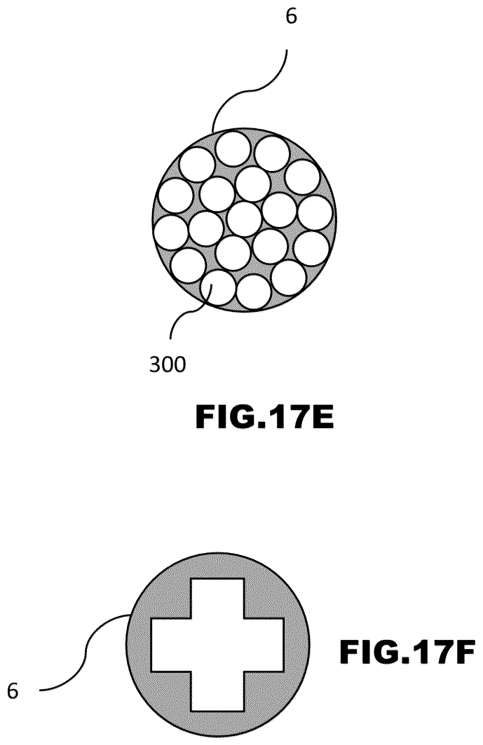

The grating 6 comprises at least one opening 300; it may comprise exactly one opening, as illustrated in FIG. 17f, or exactly two openings, as illustrated in FIG. 17a, or more.

The openings may be mostly, or all, of similar size, as illustrated in FIG. 17b, FIG. 17d or FIG. 17e. They may be of varied shape, for example square, round, or else a portion of a disk or ring, as illustrated in FIG. 17c.

The grating 6 preferably has a ratio of total surface area of the openings with respect to the contact surface with the keratin materials of greater than or equal to 0.5, thereby making it possible to benefit from relatively wide openings so as to allow the shockwave generated by the bursting of the bubbles to reach the keratin materials K over a suitable surface area.

The grating 6 may have a circular contour, as illustrated in FIGS. 17a to 17f, or a contour of another shape, for example polygonal.

The outer face of the grating 6 may be flat, and oriented for example perpendicular to the longitudinal axis X.

The grating 6 may be a single piece, formed by machining or injection molding, for example.

Its thickness may be chosen so as to give it the desired rigidity.

The grating may be made of metal, with a smooth or rough surface state on its face that comes into contact with the keratin materials. The grating is for example made of aluminum or aluminum alloy, or of stainless steel. The grating may also be formed by a rigid plastic or made of ceramic. The grating may also be composite, and comprise for example a frame embedded in a plastic matrix.

The grating may be made of a deformable and/or compressible material. The grating may thus be compressed during use thereof to a thickness that ensures a minimum distance between the emission surface and the surface of the materials to be treated.

A smooth surface state may facilitate movement over the skin in particular, while a rough surface state may exert a mechanical action that contributes to the cleaning of the skin.

Each opening in the grating may be delimited by a sharp edge, on the application face to the keratin materials. Such a sharp edge may promote cleaning, by scraping the keratin materials during the movement of the grating in contact therewith.

The device according to the invention may comprise, as illustrated in FIG. 15, a spacer 7 comprising multiple support legs 115 each extending axially over less than 360° about the axis X of the transducer, in front of the emission surface S.

The grating 6 may be carried, as in the example under consideration, by the spacer 7; in this case, the spacer may comprise a holding part 110 for the grating 6, the one or more support legs 115 acting as a rigid link between a fastening part 100 for fastening the spacer to the device and the holding part 110.

The device might not comprise a grating, but only a spacer 7 defining a contact surface with the keratin materials to be treated.

As a variant, the spacer 7 may be carried by the transducer 4 directly, with where applicable a damper between the two for limiting the transmission of vibrations from the transducer to the spacer.

The spacer 7 makes it possible to keep the transducer 4 at a distance d from the keratin materials, in order to avoid direct contact between the emission surface S and said keratin materials. When the device comprises a grating 6 carried by the spacer 7, the distance d is at least equal to the thickness of the grating 6.

The spacer 7 is preferably perforated laterally, these perforations being for example defined by the spaces between the support legs 115, as illustrated in FIG. 15, thereby making it possible to be able to observe the position of the transducer 4 and to facilitate the flow of the composition between the transducer 4 and the keratin materials.

In the example of FIG. 15, the spacer 7 comprises three support legs 115 arranged at 120° from one another, about the longitudinal axis X of the device. These support legs 115 form the link between the fastening part 100 and the holding part 110 for the grating, which are both of annular shape.

Other spacer shapes are possible.

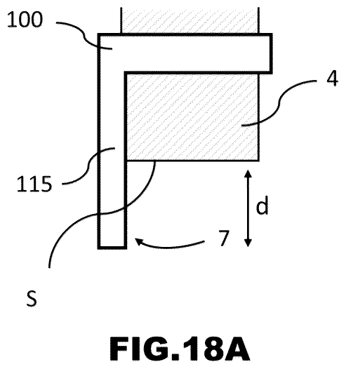

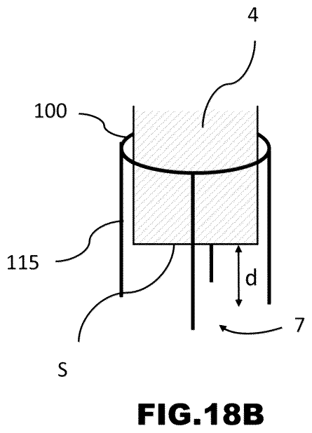

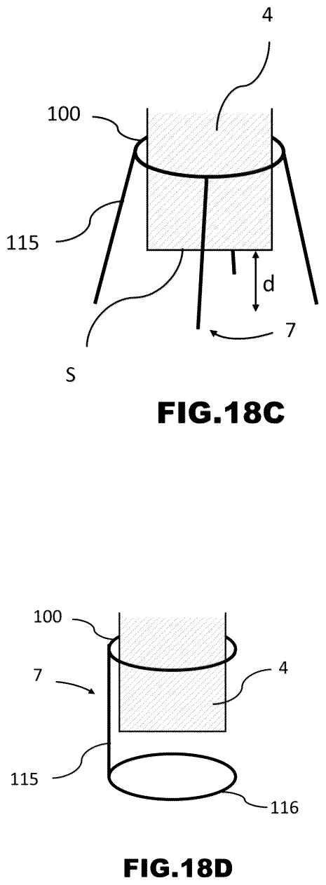

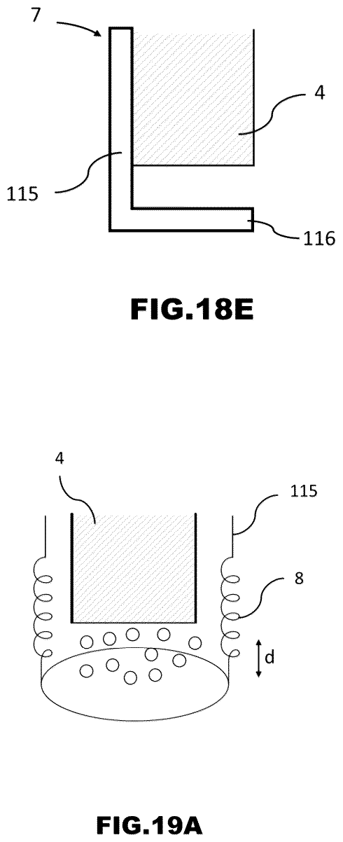

For example, the spacer 7 may comprise a single support leg 115, extending substantially perpendicular to the emission surface S of the transducer 4, as illustrated in FIG. 18a, and coming to bear directly against the keratin materials via its free end.

The spacer 7 may comprise, similarly, three or four rectilinear support legs 115 arranged at 120° or 90° from one another about the longitudinal axis X of the device. These legs may be parallel to the longitudinal axis X, as illustrated in FIG. 18b, or tilted obliquely toward the outside so as to improve the stability of the bearing of the device on the keratin materials, as illustrated in FIG. 18c.

The one or more support legs 115 may also connect to a distal part 116 extending transversely to the legs, and facing or around (in a front-on view) the emission surface S for emitting ultrasonic waves.

This distal part 116 is for example of annular shape, as illustrated in FIG. 18d, or else in the shape of a bar, as illustrated in FIG. 18e.

The one or more legs and/or the distal part may be made of a rigid material, for example metal or a rigid plastic, or of a semi-rigid or flexible material.

The spacer may be monolithic with the body of the device, or in the form of a removable nozzle.

The spacer 7, when it is in the form of a nozzle, may be fastened to the body 2 of the device by various means, for example by screwing using a screw 15, as illustrated in FIG. 15.

The axial position of the spacer may be adjustable along the longitudinal axis X of the device with respect to that of the transducer; for example, the spacer may be engaged more or less on the body of the device 2 and then the spacer may be fastened in the chosen position using the screw 15, as illustrated in FIG. 1.

This adjustable position makes it possible to precisely adjust the distance d at rest between the transducer 4 and the keratin materials, according to what is desired.

The nozzle 7 may also be extendable at the support legs 115. The legs 115 are for example each height-adjustable, the adjustment being made for example in order to adapt the position of the grating to the morphology of the keratin materials to be cleaned. This may make it possible to obliquely tilt the plane of the grating with respect to the axis X, for example.

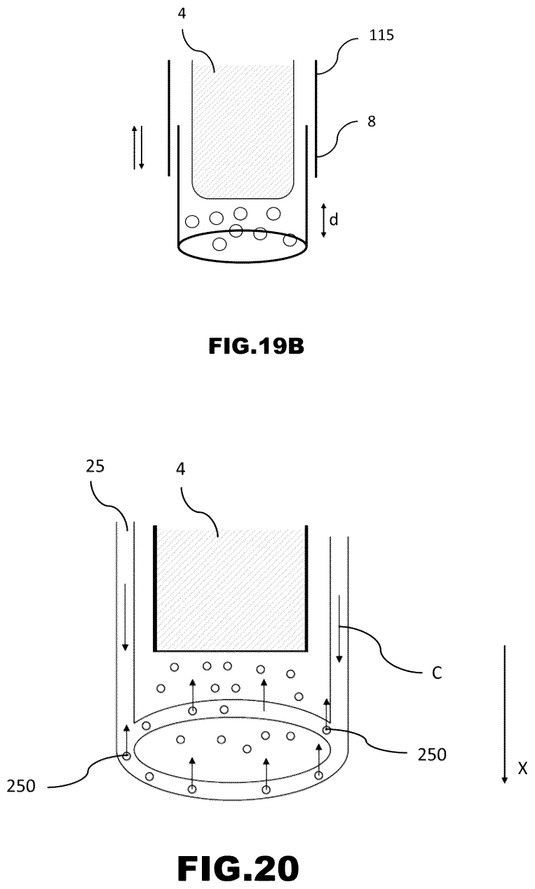

The one or more support legs 115 of the spacer 7 may also comprise an elastic return means 8, for example one or more springs, as illustrated highly schematically in FIG. 19a, or a telescopic link, as illustrated in FIG. 19b.

Such an elastic means 8 makes it possible to vary the spacing d between the transducer 4 and the keratin materials K on the basis of the pressure with which the spacer is pressed against the keratin materials K to be cleaned, and/or to maintain a certain application pressure when moving the device, and for example to adapt to any reliefs of said materials.

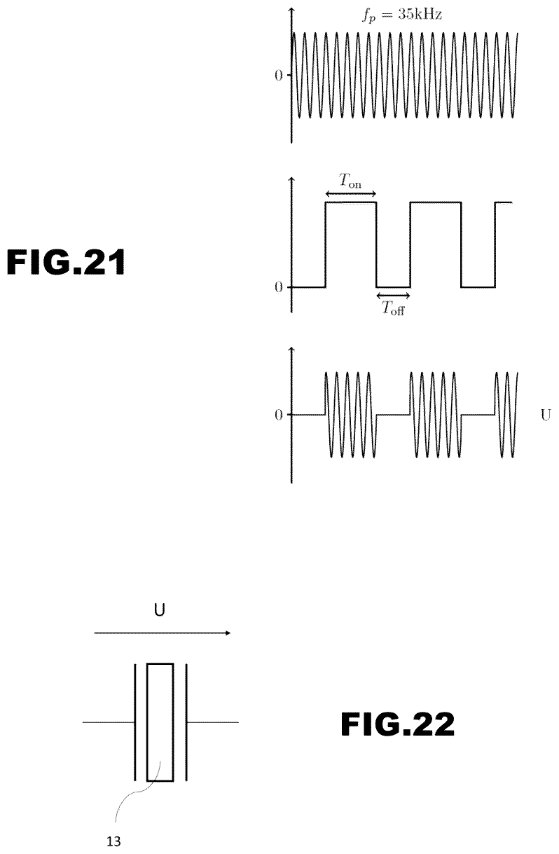

As illustrated in FIG. 20, the device 1 may comprise one or more supply ducts 25 passing through the spacer 7 from a composition tank 22, in particular by extending along the one or more support legs 115, and opening out via at least one outlet 250, located close to the area to be treated during use.

The one or more supply ducts 25 may extend in the grating and/or the support part 110 for the grating 6, when this is carried by a spacer 7. The device 1 comprises for example multiple outlets 250 located on the grating 6 or on the support part 110 for the grating 6 (not shown).

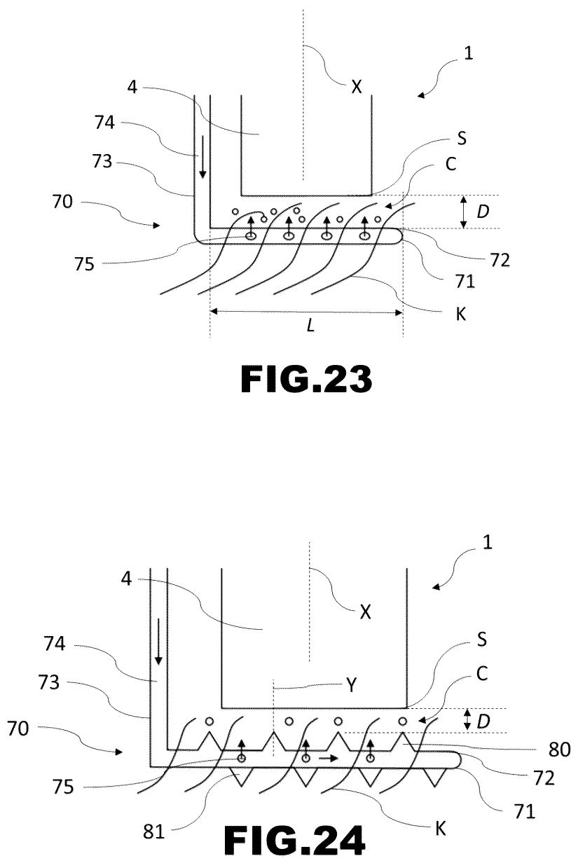

FIG. 23 illustrates a treatment device 1 according to the invention comprising a transducer 4, having an emission surface S for emitting acoustic waves into a cosmetic composition C, and a guiding member 70 for guiding a strand of hair K, allowing the hair K to be guided as it passes through the treatment area.

The treatment device 1 may be in the form of a handpiece, not shown, that operates autonomously or is connected to a base station.

The transducer 4 may be fastened removably or non-removably to the device.

The guiding member 70 may be made of one or more parts, these parts being able to be disassembled and/or assembled and/or replaced and/or modified by the user.

The transducer 4 is powered by an electrical generator, not illustrated in this figure, which may form part of the device 1, being for example integrated into the handpiece or connected thereto by a flexible cable.

The guiding member 70 may be mounted removably or non-removably on the rest of the device 1, and be mobile or immobile with respect thereto, for example between a use configuration in which the member 70 guides the hair K and a disengaged configuration in which it is spaced from the transducer 4 and allows the strand of hair K to be inserted. It may also be mounted fixedly on the device 1, forming a passage for inserting the strand of hair K to be treated.

The guiding member 70 may be arranged so as to define a space with the emission surface S through which the hair K passes during treatment, and extend at least partially facing the emission surface S as illustrated, having for example a guiding portion 71 defining a guiding surface 72 substantially parallel to the emission surface S.

The guiding surface 72 may be flat or have another shape tailored to the movement over the strand, for example a circular shape.

The guiding surface 72 may have a width L greater than or equal to that of the emission surface, for example have a width L of between 1 mm and 180 mm, the emission surface having for example a width of between 1 and 150 mm.

The guiding member 70 may comprise a support portion 73 that extends for example along the transducer 4 and is connected to the body of the device 1 at an end opposite the guiding portion 71 by any appropriate means.

The guiding member 70 may advantageously serve to supply the cosmetic composition C to the treatment area. To this end, the guiding member 70 may be passed through by at least one duct 74 that opens out, via one or more orifices 75, in the guiding portion 71 facing the emission surface S.

The duct 74 is connected to a fluidic circuit that ensures for example the flow of the composition by taking it from a tank.

The distance D between the emission surface S and the guiding surface 72 is for example between 0.1 mm and 30 mm.

This distance D may be fixed, or adjustable, by virtue for example of specific mounting of the guiding member 70 on the device 1, allowing the user to move the guiding member 70 relative to the transducer 4 as required.

The orifices 75 are for example spaced regularly along the guiding portion 71, as illustrated, but, as a variant, may be spaced differently.

The orifices 75 are for example circular, but may also be produced in another shape, for example in the shape of one or more slots.

To use the device 1 of FIG. 23, the user may engage a strand of hair K between the guiding portion 71 and the transducer 4, and move the device 1 along the strand of hair K, for example starting from the root of the hair to the tips.

During this movement, the composition C is delivered to the treatment area by the device 1, in particular via the distribution orifices 75.

The composition C present in the treatment area, in contact with the hair K, is subjected to the action of the acoustic waves emitted by the transducer 4.

Bubbles are generated by cavitation in the treatment area, and then burst, creating shockwaves that contribute to the cleaning and/or the bleaching of the hair K.

The composition C delivered to the treatment area may be drained away from the device or recycled.

The guiding member 70 may also be formed so as to comb the hair K upstream or downstream of the treatment area, or even in said treatment area. This may make it possible for example to have a strand of more homogeneous thicknesses facing the emission surface S, the strand engaged in the device being divided into smaller, easier-to-access strands by teeth and/or bristles and/or etchings and/or micro-reliefs.

FIG. 24 shows a variant of a guiding member 70 that makes it possible to carry out such combing and that differs in this respect from the guiding member 70 of FIG. 23 through the presence of at least one row of teeth 80, shown schematically.

These teeth 80 extend for example, as illustrated, with their longitudinal axis Y parallel to the longitudinal axis X of the transducer 4.

The teeth 80 may extend facing the emission surface S, as illustrated, or, as a variant, are offset relative to this surface so as to comb the hair K upstream or downstream thereof.

The guiding member 70 may comprise both teeth 80 and orifices 75 for distributing the composition C, as illustrated.

These orifices 75 open out for example between the teeth 80.

The guiding member 70 may comprise combing and/or guiding reliefs elsewhere than facing the emission surface S, for example on the opposite side, as illustrated. FIG. 24 thus shows a row of additional teeth 81 opposite the teeth 80.

The height of the teeth 80 and 81 of the combing member 70 of FIG. 24 is for example of the order of 0.5 mm to 20 mm.

Of course, the invention is not limited to a particular tooth shape.

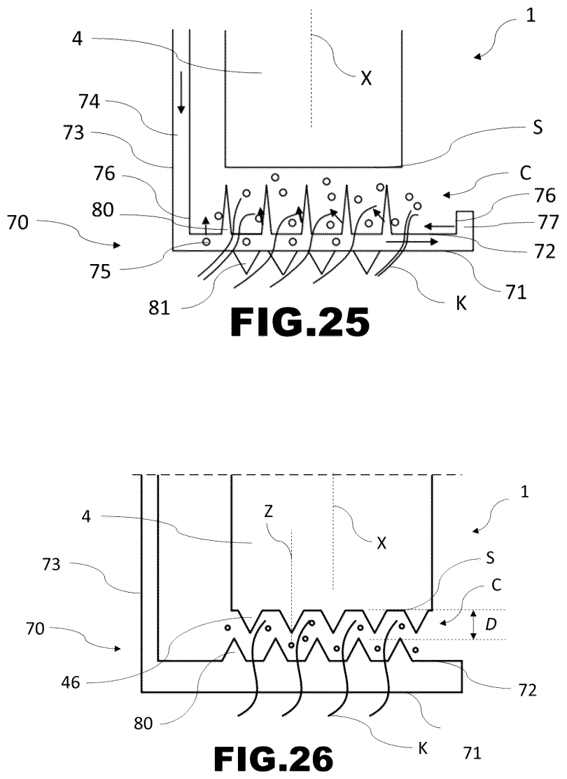

In the example of FIG. 25, the teeth 80 are hollow and in communication with the duct 74 so as to distribute the composition C via orifices formed in the teeth, not visible in this figure.

The guiding member 70, as illustrated, may retain distribution orifices 75 on the guiding portion 71 that carries the teeth 80 and 81. As a variant, the composition C is distributed only via the orifices provided on the teeth 80.

FIG. 25 also illustrates the possibility of the guiding member 70 having one or more lateral guiding surfaces 76, between which the strand of hair is held when it passes through the treatment area.

For example, the device comprises one lateral guiding surface 76 defined by the support portion 73 and another one defined by a return 77 that is directed inward.

Where applicable, as illustrated, the return 77 is hollow and communicates with the duct 74 so as to distribute the composition C via at least one orifice (not visible) opening out in the direction of the treatment area.

The emission surface S of the transducer 4 may also have teeth 46 and/or bristles 47 and/or etchings and/or micro-reliefs and/or spikes, in particular metal ones, so as to comb the hair K upstream or downstream of the treatment area, or even in said treatment area. The emission surface S of the transducer 4 may comprise the same type of reliefs as the guiding member 70 or, as a variant, different reliefs.

FIG. 26 schematically shows a variant device 1 in which the emission surface S comprises teeth 46 or other reliefs for contributing to the combing and the guiding of the strand of hair K.

These teeth 46 or other reliefs extend for example, as illustrated, with their longitudinal axis Z parallel to the longitudinal axis X of the transducer 4.

The teeth 46 or other reliefs may be arranged, as illustrated, so as to fit between the teeth 80 of the guiding member 70.

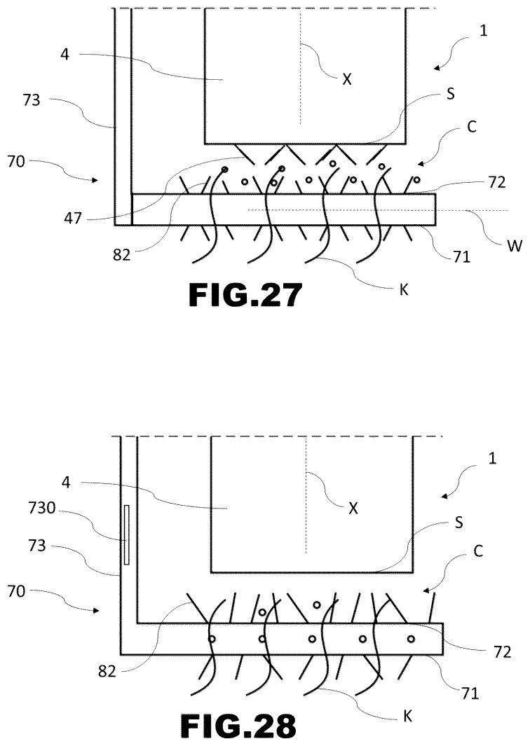

FIG. 27 schematically shows a variant device 1 that also makes it possible to carry out combing and that differs from the device 1 shown in FIG. 26 by the presence of bristles 47 on the emission surface S and bristles 82 on the guiding member 70, the bristles 47 and 82 possibly being natural and/or synthetic.

The emission surface S of the transducer may be defined by a removable and replaceable part, so as to offer various solutions for guiding and/or combing the hair K depending on the guiding member 70 that is used.

The guiding member 70 and the transducer 4 may be mounted on the device 1 so as to be mobile with respect to one another and/or with respect to the device 1; they are for example moved longitudinally, transversely, vibrate and/or rotate.

In particular, the guiding portion 71 of the guiding member 70 may be rotational about its longitudinal axis W, as illustrated in FIG. 27, being driven for example by a motor or free to rotate. The guiding portion 71 may rotate, facing the emission surface S, while the strand of hair K is passing between the guiding member 70 and the transducer 4, this making it possible for example to guide and/or to comb the strand of hair K as it passes through.

The transducer 4 of FIG. 28 may rotate about its longitudinal axis X. The guiding member 70 and the transducer 4 of FIG. 28 may also be moved longitudinally along the axis X so as to move the guiding surface 72 and emission surface S toward or away from one another, for example in order to adapt the treatment to the thickness of the strand of hair K. The guiding member 70 may in particular be returned to a rest position using an elastic return means 730, for example a spring, carried by the support portion 73.

The device 1 may comprise a spacer element 7, also called spacer, as mentioned above.

This spacer element 7 may make it possible to keep the hair K at a predefined distance from the emission surface S. This spacer element 7 may or may not form part of the guiding member 70.

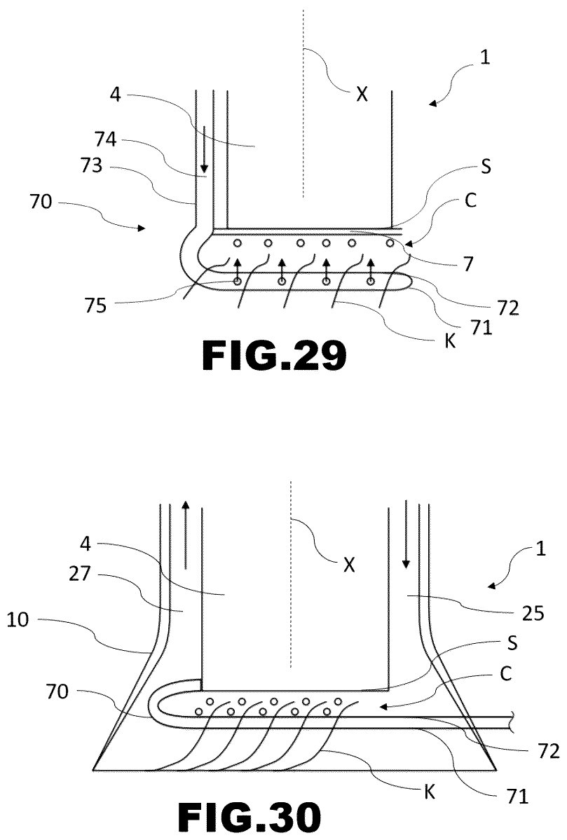

For example, the spacer element 7 is carried by the guiding member 70, being for example fastened to the support portion 73, as illustrated in FIG. 29.

The spacer element 7 is permeable to the acoustic waves emitted by the transducer 4, and defines, with the guiding portion 71, the treatment area through which the hair K passes in order to be treated.

The spacer element 7 is for example in the form of a grating, for example made of metal material or of plastic.

FIG. 29 illustrates the distribution of the composition C via orifices 75 of the guiding member, but, as a variant, the composition C may be supplied to the treatment area by any other means, or even already be present on the hair K inserted into the treatment area.

The variant device of FIG. 30 comprises a treatment head 10 that ensures a flow of the composition C between a supply duct 25 and a return duct 27.

The composition is thus able to flow in the treatment area between the ducts 25 and 27.

The flow of the composition is ensured for example by an electric or manual pump, not shown.

The treatment head 10 may comprise a sealing nozzle, not illustrated, which is applied to the hair around the treatment area in order to limit losses of composition during the treatment. This sealing nozzle may be applied to a bearing surface, not illustrated, which is lowered onto the treatment head 10 during use of the device 1, the treatment head 10 being for example carried by a first jaw of the device 1 and the bearing surface being carried by a second, opposing jaw, articulated on the first, in the manner of a straightening iron.

The guiding member 70 may extend at least partially within the treatment head 10 during use, as illustrated schematically in FIG. 30, in order to keep the hair K to be treated in the treatment area.

The hair K may in particular be kept, by the guiding portion 71 of the guiding member 70, at a distance from the emission surface S less than or equal to a predefined value.

The guiding member 70 may be carried by the abovementioned second jaw.

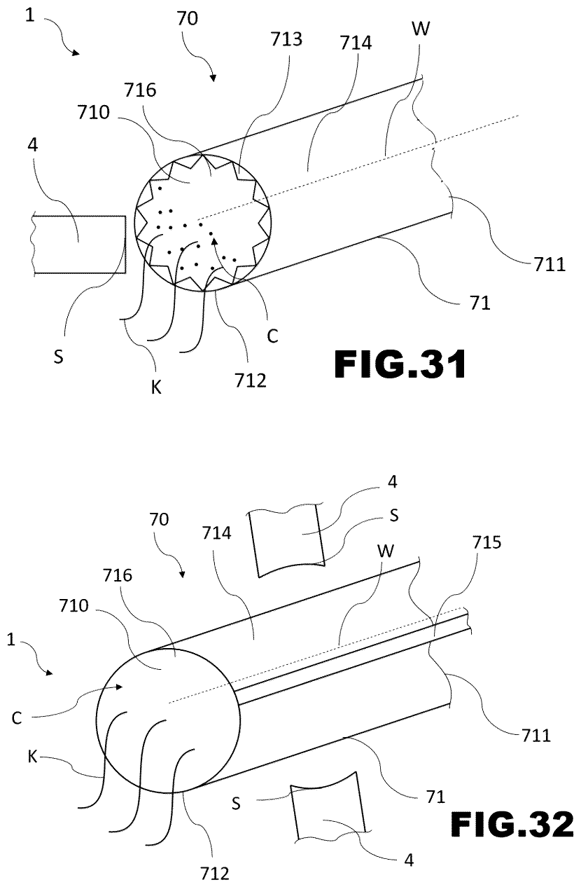

The guiding member 70 may, as illustrated in FIG. 31, comprise a hollow guiding portion 71, for example of tubular shape, defining a cavity 710 for guiding the hair K to be treated, which cavity is in particular open at two opposing ends 711 and 712.

The cavity 710 may have a constant or non-constant cross section.

In this example, the strand of hair K is inserted into the cavity 710 via the end 711 and exits it via the opposite end 712.

The inner surface 716 of the cavity 710 may have reliefs that contribute to combing and/or holding the hair. These reliefs may be in the form of ridges or teeth 713.

The guiding portion 71 may be deformable, and in particular may have its diameter varied under the action of an adjustment means of the device, for example using a clamping member such as a hose clamp, to adapt the device to the strand of hair and to the desired treatment.

The guiding portion 71 may be permeable to the acoustic waves. As a variant, it may form all or part of the transducer, in particular of the sonotrode.

The device may be configured such that the transducer 4 is able to move along the guiding portion 71, for example with its emission surface S facing the outer surface 714 of the guiding portion 71, as illustrated. The transducer 4 is moved for example in a motorized manner, the transducer 4 making for example round trips along the guiding portion or being moved manually by the user.

The transducer 4 may also rotate about the guiding portion 71, while preferably keeping its emission surface S facing the outer surface 714 of the guiding portion 71. The rotation of the transducer may be motorized or performed manually by the user, for example in order to adjust the side of the hair that is treated.

The guiding portion 71 may also be mobile, for example with a movement thereof along its longitudinal axis W, or with a rotation about its longitudinal axis W. The movement of the guiding portion 71 may or may not be motorized.

The device 1 may comprise, where applicable, multiple transducers 4, in particular two transducers 4, as illustrated in FIG. 32. These two transducers 4 may be diametrically opposing. They may be fixed with respect to one another and to the guiding portion 71, or mobile with respect thereto, and for example be moved or movable independently of one another.

The presence of multiple transducers makes it possible to increase the effectiveness of the device 1, for example by making it possible to simultaneously treat two opposing faces of the hair and/or to obtain a greater bubble density.

The emission surface S of the one or more transducers 4 may have a concave shape toward the guiding portion, so as for example to match the outer surface 714 of the guiding portion 71 as best possible, as illustrated in FIG. 32.

The guiding member may, as illustrated in this figure, have a slot 715 along the portion 71, between the two ends 711 and 712. This slot 715 may make it possible to insert the strand of hair K into the cavity 710 more easily.

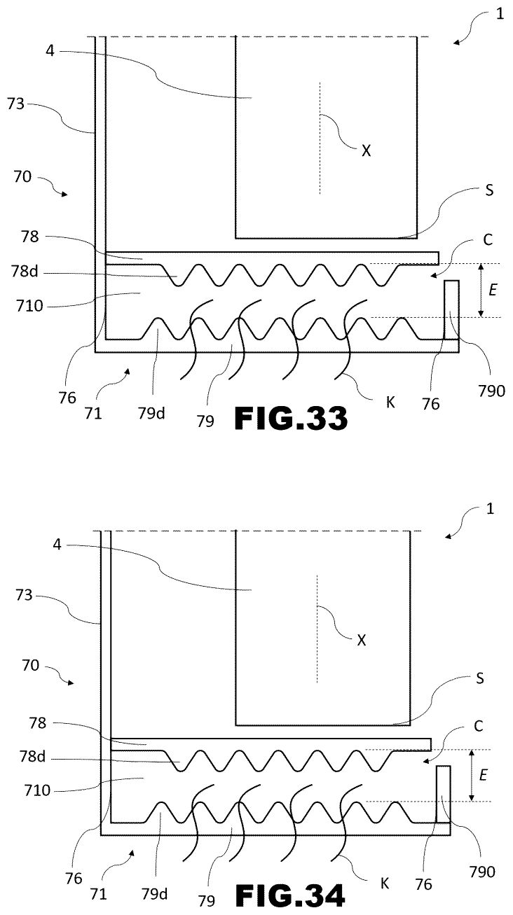

The cavity 710 may thus be defined by a guiding portion 71 comprising two parts 78 and 79 that are mobile with respect to one another, in particular in the form of jaws, as illustrated in FIG. 33, said parts being moved between a spaced configuration for inserting a strand of hair K between them and a close configuration for treating the hair. The jaws 78 and 79 may be carried by the support portion 73 for the guiding member 70, as illustrated. They may be fastened removably or non-removably to the support portion 73.

The distance E between the two jaws 78 and 79 may vary between 0 mm in the close configuration and 50 mm in the spaced configuration.

The two jaws 78 and 79 may be moved with respect to one another, along the axis X, in order to increase or reduce the distance E, for example in order to adapt the treatment to the thickness of the strand of hair K to be treated.

Each jaw 78 or 79 may have, on its surface facing the other jaw, teeth 78d and 79d and/or bristles or other reliefs, making it possible in particular to divide the strand of hair K in order to treat it better.

The jaw 78 that is proximal with respect to the transducer 4 is preferably permeable to the acoustic waves emitted thereby and may to this end comprise holes, for example produced in the form of a grating or else formed from a specific material permeable to the acoustic waves.

As illustrated in FIG. 33, the guiding member 70 may have two lateral guiding surfaces 76, one of which is defined by the support portion 73 and the other of which is defined by a return 790 of the lower jaw 79 that is directed inward.

The space between the two jaws 78 and 79, as a variant, is closed on the side opposite the support portion 73 by a closing portion 791, as shown in FIG. 34.

In the example of this figure, the support portion 73 and the closing part 791 comprise for example at least one elastic return member 500, for example one or more helical springs, allowing the jaws 78 and 79 to move toward one another and urging them to a rest position, for example the close configuration or the spaced configuration depending on the variant.

### EXAMPLES OF TREATING STRANDS OF HAIR

### Example 1

Confocal microscopy analysis is carried out on natural strands in order to study, using the passive diffusion of a fluorochrome in the fiber, whether the integrity of the cuticle is altered by the treatment device.

Three hair samples are prepared, namely:
sample 1: a strand of undyed and untreated natural hair serving as reference,
sample 2: a strand of the same undyed natural hair treated with two passes of the treatment device according to the invention, emitting ultrasound at a frequency of 33.4 kHz, in contact with a cosmetic composition C such as a foam containing soap-type surfactants (partially neutralized long-chain fatty acid),
sample 3: a strand of the same undyed natural hair treated with ten passes of the treatment device according to the invention, emitting ultrasound at a frequency of 33.4 kHz, in contact with the same composition C.

The three strands of hair are then stained for the microscopic study with a hydrophilic fluorochrome, specifically fluorescein.

Sections of the strands are then observed using laser scanning confocal microscopy in order to study the diffusion of the fluorescein within the hair.

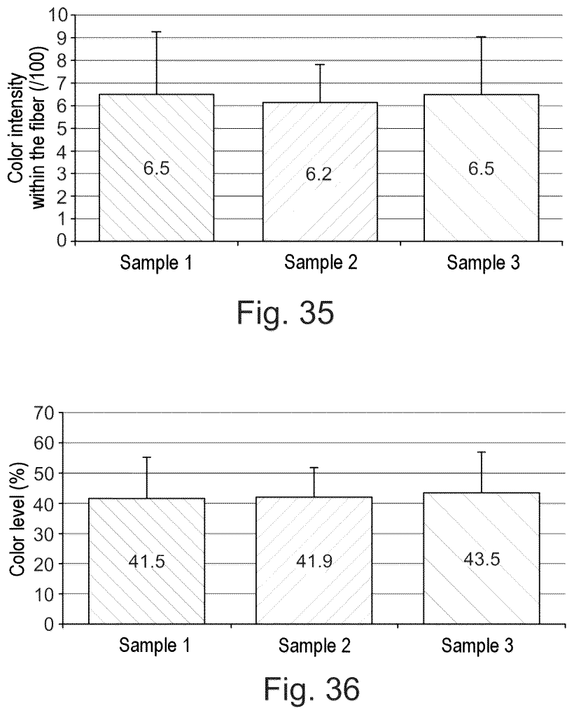

The images obtained are analyzed using software. A first analysis is carried out on the color intensity within the fiber; the results may be seen in FIG. 35.

A second analysis is carried out on the fluorescein color level. The results may be seen in FIG. 36.

It is observed that the staining intensity within the hair fiber is low, between 6.2% and 6.5%, and of the same order of magnitude for the hair serving as reference and the hair treated using ultrasound according to the invention. This signifies that the integrity of the cuticle of the treated hair has not been altered. Ultrasound does not damage the capillary sheath.

### Example 2

Transmitted light analysis is carried out on dyed strands in order to visualize the decrease in thickness and to quantify the dye intensity within the fiber of the treated hair.

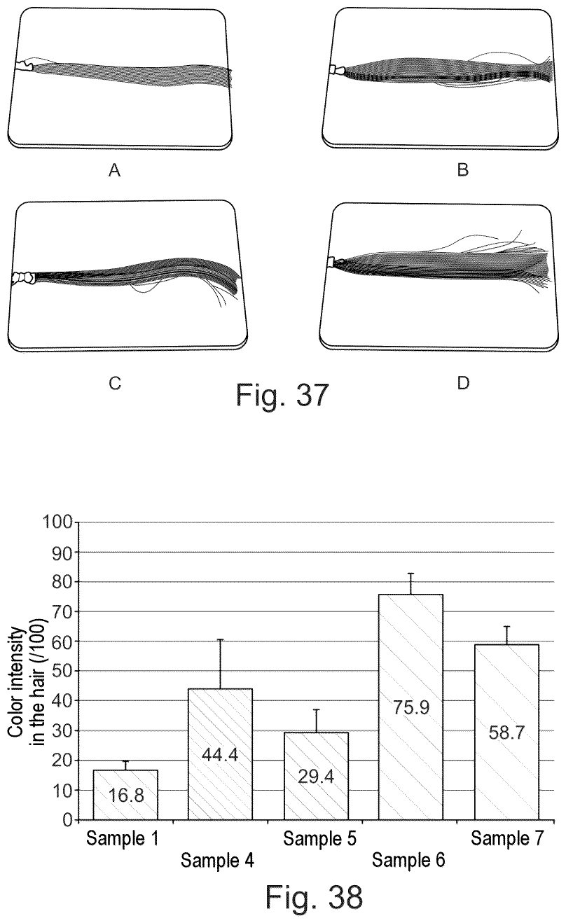

Five hair samples are prepared, namely:
sample 1: a strand of undyed and untreated natural hair serving as reference,
sample 4: a strand of the same hair dyed using the product “Colorista Washout L'Oreal Paris” and not treated, visible in FIG. 37A,
sample 5: a strand of the same hair dyed using the product “Colorista Washout L'Oreal Paris” and treated with ten passes of the treatment device according to the invention, emitting ultrasound at a frequency of 33.4 kHz, visible in FIG. 37 B,
sample 6: a strand of the same hair dyed using the dye “Majirouge 6.66 L'Oreal Pro” and not treated, visible in FIG. 37 C,
sample 7: a strand of the same hair dyed using the dye “Majirouge 6.66 L'Oreal Pro” and treated with ten passes of the treatment device according to the invention, emitting ultrasound at a frequency of 33.4 kHz, visible in FIG. 37 D.

Sections of the strands are observed using laser scanning confocal microscopy in transmitted light.

The following observations are made on the grayscale images obtained using confocal microscopy:
the undyed and untreated hair of the reference strand (sample no. 1) have a light gray uniform hue,
the hair dyed using the product “Colorista Washout L'Oreal Paris” and not treated (sample no. 4) mostly exhibits coloring on the surface with a more or less thick dark ring that is however mainly located on the periphery,
the hair dyed with the dye “Majirouge 6.66 L'Oreal Pro” and not treated (sample no. 6) exhibits far darker and deeper coloring since the hair is almost completely dyed,
the hair treated with ultrasound (samples no. 5 and 7) exhibits less intense coloring with respect to its respective dyed reference, thereby demonstrating the benefit of the treatment for bleaching dyed hair while minimizing the impact on the integrity of the hair.

The analysis is carried out by quantifying the average coloring intensity of the hair based on a grayscale from 0 to 100, with 0=white and 100=black. The results of the analysis may be seen in FIG. 38.

After 10 ultrasonic passes over dyed strands of hair, we observe a decrease in coloring of 34% for the product “Colorista Washout L'Oreal Paris” and of 23% for the dye “Majirouge 6.66 L'Oreal Pro”.

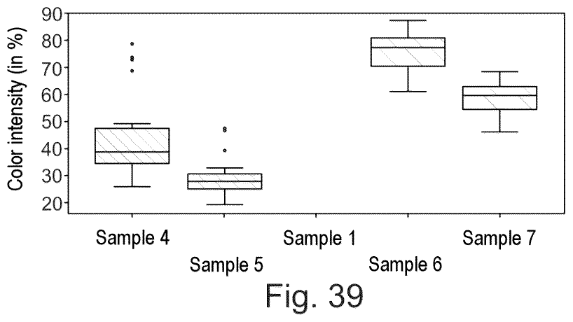

Statistical analysis of the coloring data was also carried out, reproduced in FIG. 39. This analysis confirms that the difference between the measured average values is statistically different within a confidence interval of 95%.

Of course, the invention is not limited to the examples that have just been described.

It is thus possible to give the guiding member even more shapes.

The device may be used in the following steps, which are given by way of example for a device comprising one or more removable tanks, a transducer or a removable sonotrode, and/or a treatment head possibly equipped with additional elements such as a spacer, grating, etc.:
The user selects the fluid, in particular the cosmetic composition, according to the treatment that they wish to perform and fills the tank, or inserts a pre-filled tank (for example a single-use tank) into the handpiece.
The user selects the type of sonotrode and the elements in contact with the keratin materials to be used according to the area that they wish to treat and fastens them to the rest of the device.
The user turns on the device using the on/off button and selects a program that may be viewed on a screen.
The starting of the program may be associated with an acoustic signal, a vibration and/or an indicator light.
The user applies the treatment head in contact with the keratin materials, thereby triggering, by virtue of a contact sensor, the starting of the pump and of the transducer.
The pump generates a vacuum for allowing a flexible lip arranged on the end of the treatment head to stick in contact with the keratin materials, and making it possible to create a closed space between the emission surface for emitting ultrasonic waves and the surface of the materials to be treated.
The fluid is set in flow in the space thus created. The transducer generates acoustic waves that lead to the cavitation of the bubbles in the treatment area.
The used fluid is recovered in a second tank, or a second compartment of the tank; it may be filtered and reintroduced into the fluidic circuit, thus making multiple passes through the treatment area.
The user views the soiling level of the recovered fluid. The user may empty the tank containing this fluid at the end of their treatment or earlier if necessary.
The device stops working as a safety precaution when the user removes the treatment head from the keratin materials, in the event of detection of an excessive temperature increase, or when it is detected that the device is static.
Once the treatment has ended, the user may replace the device on a base in order to recharge it, or remove the battery in order to recharge it separately.
The user may also launch a cleaning program for the device by filling the tank with a specific product and by placing the device on a dedicated surface for closing the fluidic circuit for cleaning.

### Examples of Treating Skin

Samples of artificial skin (from Bioskin) are prepared, having applied to them a stubborn foundation over a thickness of around 6 μm+/−20%.

Drying is carried out for 20 min minimum at room temperature. As a variant, drying may be carried out for 15 min at room temperature and drying may be completed for 2 min using a hairdryer.

The composition is for example one of the compositions C1 to C5 mentioned below.

The acoustic waves are generated by an ultrasonic transducer 13 excited by a sinusoidal electrical signal U that has a frequency of around 34 kHz, is pulse-width modulated, as illustrated in FIG. 21, and delivered by a generator G.

The amplitude of the voltage U may be adjusted by the user during the tests by acting on the generator.

The pulses are characterized by a pulse duration Tₒₙ and a duty cycle

$$α = \frac{{T}_{on}}{{T}_{on} + {T}_{off}} ,$$

where T_{off} is the duration of the “low” or “passive” state over one period.

A sonotrode 4 as illustrated in FIG. 1 is used for example to emit the acoustic waves, which sonotrode is moved slowly in contact with the composition, without touching the film of foundation.

The sonotrode comprises for example a ceramic piezoelectric transducer system.

The nominal frequency of the transducer is for example around 34 kHz.

The sonotrode is for example made of titanium.

The transducer comprises for example ceramics separated by an insulator and mounted on one another by clamping.

The diameter of the sonotrode is for example around 1.8 cm, that is to say a surface area of around 2.5 cm², and the ceramics of the transducer have for example a diameter slightly smaller than that of the sonotrode.

The sonotrode is for example contained within a casing, with its front end projecting a few centimeters beyond the casing, preferably less than 5 cm.

Tests are carried out by varying various parameters, namely:
the total concentration of surfactant(s) (%) in the composition,
the peak acoustic intensity I (in W/cm²), which depends directly on the activation voltage U
the pulse duration Tₒₙ
the duty cycle, Ton/Toff
the duration for which the surface to be cleaned is subjected to the acoustic waves,
the distance between the emission surface and the surface to be cleaned.

The acoustic intensity I_{SATA} on the surface to be cleaned, expressed in W/cm², is calculated on the basis of the selected parameters. It is given by:

$${I}_{SATA} = I \cdot \frac{{T}_{on}}{{T}_{off}}$$

The acoustic intensity I_{SATA} may also be expressed as a function of the average acoustic pressure pₘ applied to the treated area:

$${I}_{SATA} = \frac{{p}_{m}^{2}}{ρ c}$$

Where ρ is the bulk density of the composition (in kg·m-3), c is the speed of sound in the composition (in m·s-1), pₘ is the average acoustic pressure (in Pa, or equivalently in N·m-2 or kg·m-1·s-2), and I_{SATA} in W·m-2.

The effectiveness of the combination of parameters for each test is evaluated on the basis of a make-up removal percentage (% make-up removal) calculated based on gradients measured on a colorimetric scale (delta E).

It is considered that the cleaning has a satisfactory effect for a delta E greater than or equal to 15, and a make-up removal percentage greater than 65%.

TABLE 1

| Surfactant | Intensity I | | Duty cycle | Duration | Isata | % make-up | |
|---|---|---|---|---|---|---|---|
| (%) | (W/cm²) | Tₒₙ | Tₒₙ/T_{off} | (s) | (W/cm²) | removal | Delta E |
| 0.5 | 6.8 | 0.05 | 58.33 | 3 | 3.96 | 66.6 | 16.15 |
| 0.5 | 7.2 | 0.02 | 66.66 | 4 | 4.78 | 81.9 | 18.54 |
| 0.5 | 6.2 | 0.08 | 66.66 | 4 | 4.13 | 86.1 | 16.97 |
| 0.8 | 6.8 | 0.04 | 56 | 3 | 3.66 | 86.3 | 18 |
| 1 | 6.8 | 0.05 | 50 | 3 | 3.40 | 72.5 | 15.07 |
| 1 | 6.8 | 0.05 | 58.33 | 3 | 3.96 | 74.9 | 16.27 |
| 1 | 6.8 | 0.05 | 58.33 | 2 | 3.96 | 77.8 | 15.92 |
| 1 | 6.8 | 0.05 | 58.33 | 3 | 3.96 | 78.7 | 18.15 |
| 1 | 6.8 | 0.05 | 58.33 | 3 | 3.96 | 79.3 | 17.29 |
| 1 | 6.8 | 0.05 | 62 | 3 | 4.21 | 81 | 17.51 |
| 1 | 6.8 | 0.05 | 58.33 | 3 | 3.96 | 84.5 | 16.72 |
| 1 | 7.0 | 0.05 | 58.33 | 3 | 4.18 | 88.4 | 17.8 |
| 1 | 6.8 | 0.05 | 58.33 | 4 | 3.96 | 90.4 | 19.98 |
| 1 | 6.8 | 0.02 | 58.33 | 3 | 3.96 | 91.5 | 18.09 |
| 1.2 | 6.8 | 0.04 | 56 | 3 | 3.80 | 75.6 | 16.21 |
| 1.5 | 6.2 | 0.08 | 50 | 4 | 3.10 | 79.8 | 19.36 |
| 1.5 | 6.2 | 0.02 | 66.66 | 4 | 4.13 | 89.7 | 18.46 |

For the combinations of parameters summarized in the above table, good removal of the foundation is observed in the regions where the sonotrode passes while active.

Great difficulty is also observed in terms of removing the make-up simply by passing a sponge or brush over it, thereby highlighting the cleaning effect obtained in the invention.

### Response Surfaces

It is possible to expand the values of parameters obtained experimentally above and demonstrating good make-up removal effectiveness to ranges of parameters using an optimization plan that takes into account the various interactions between all of the parameters.

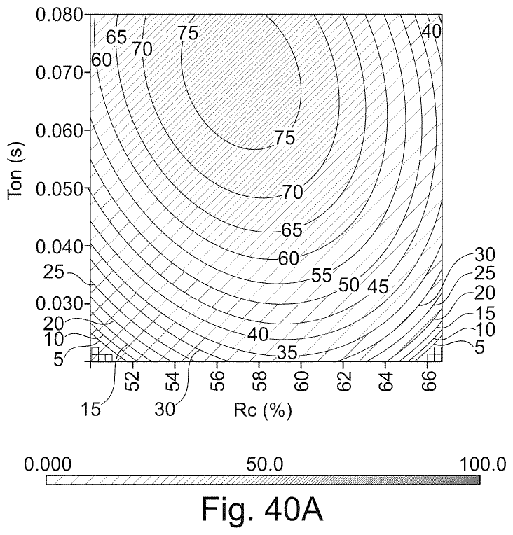

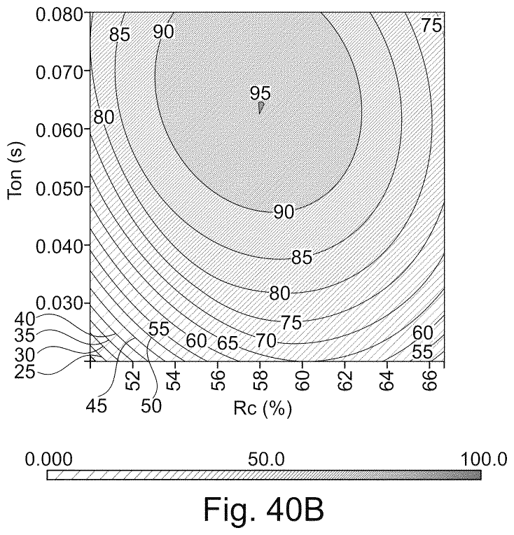

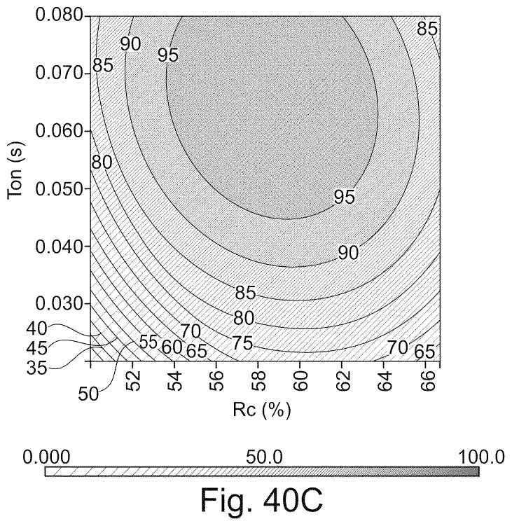

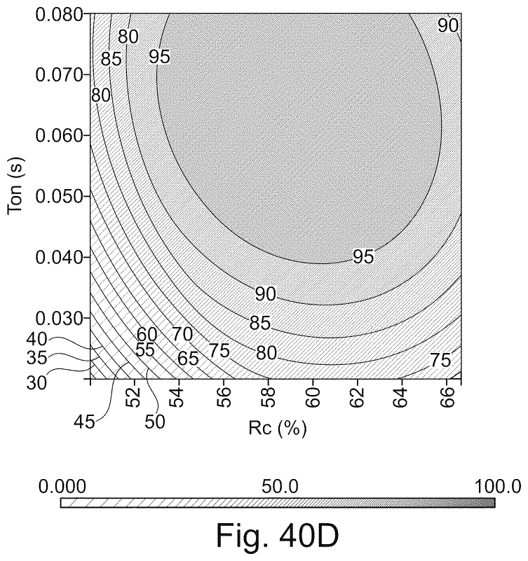

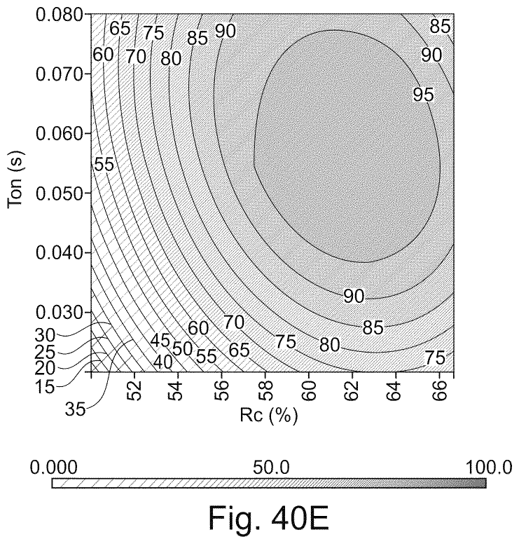

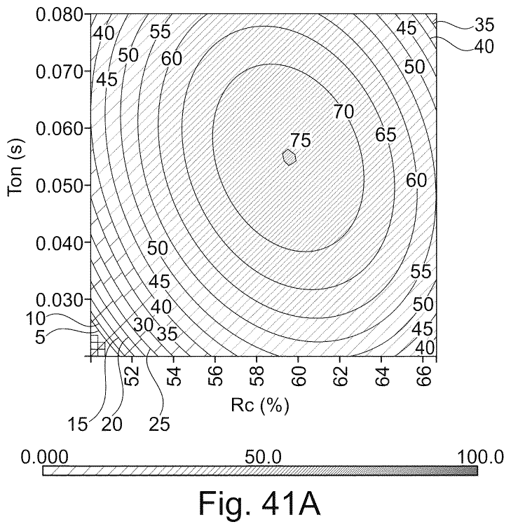

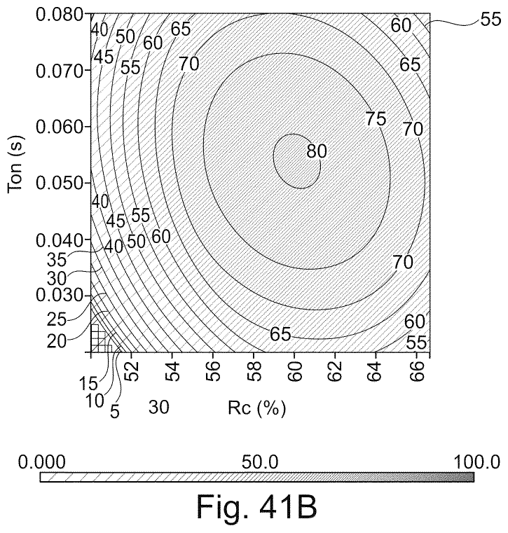

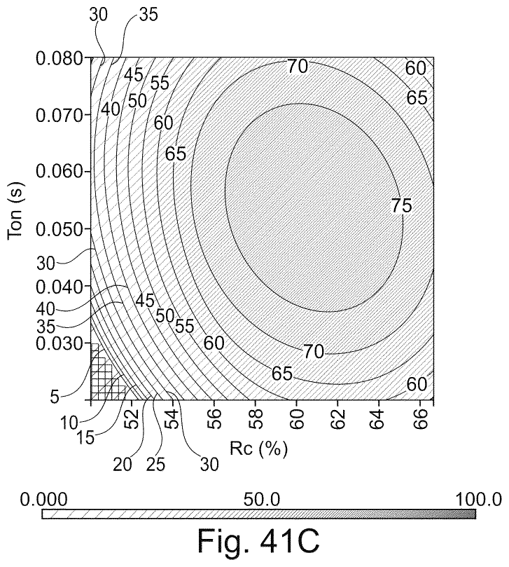

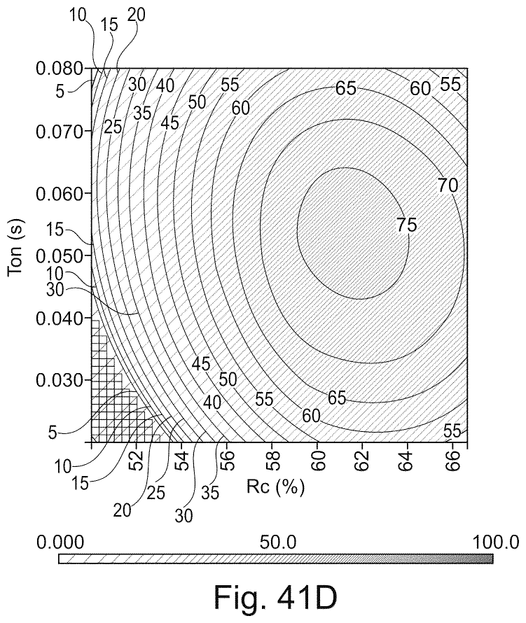

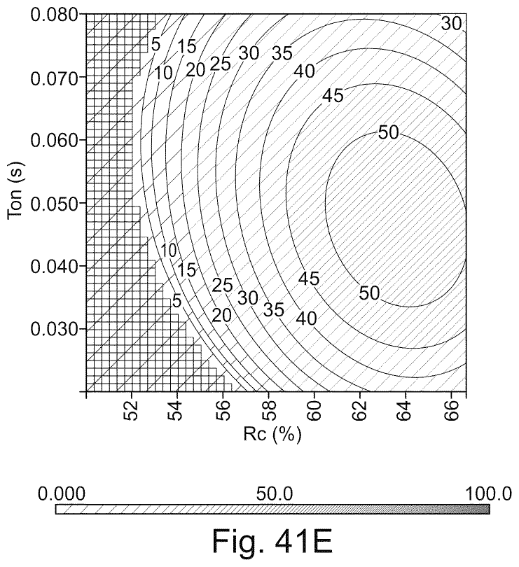

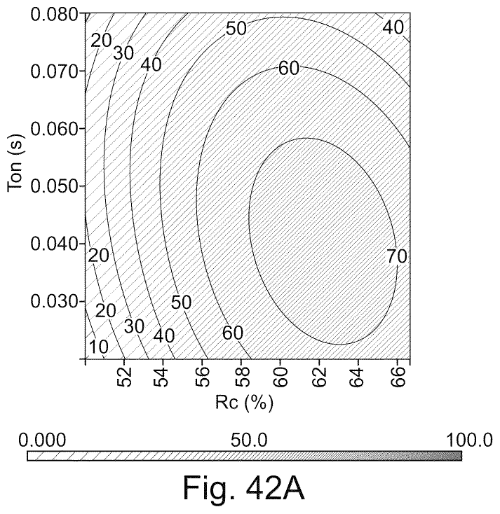

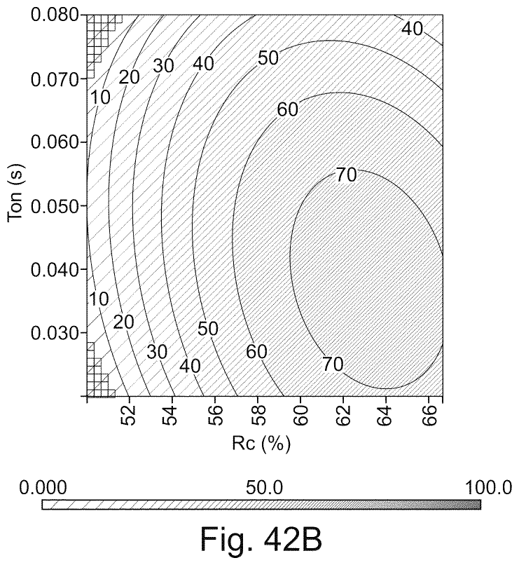

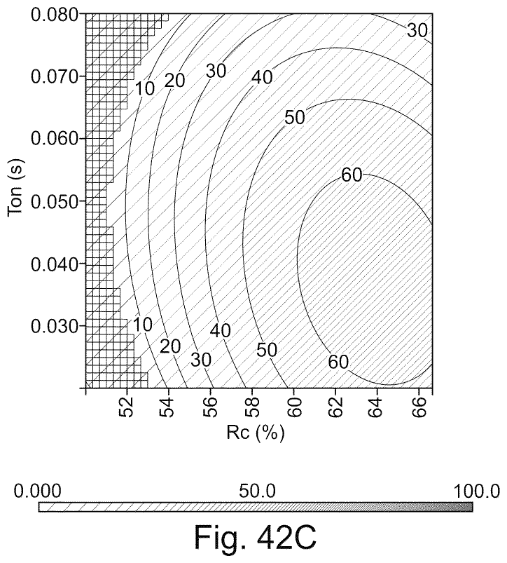

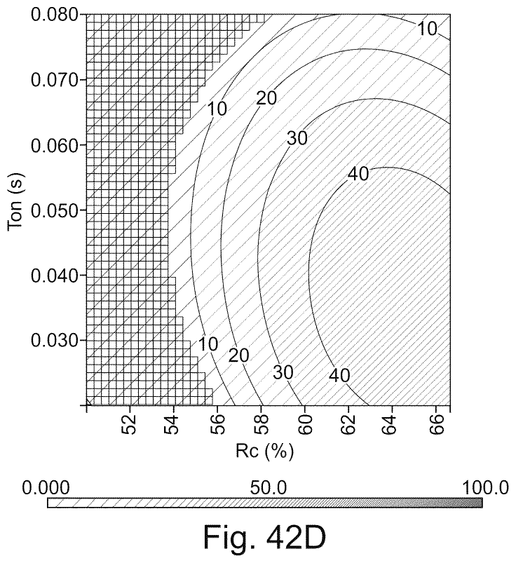

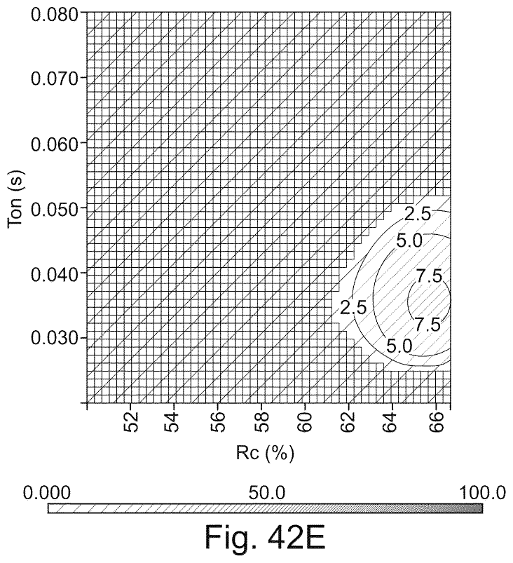

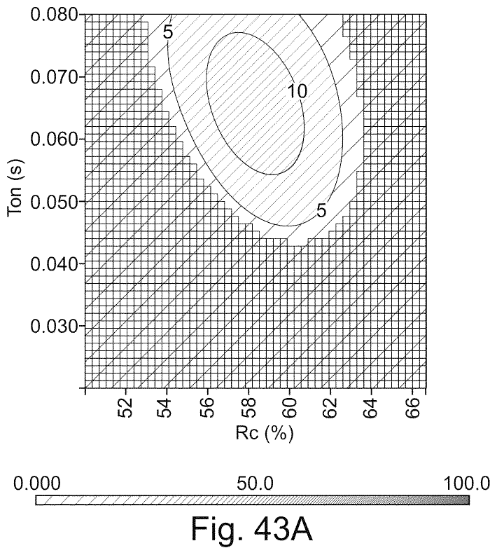

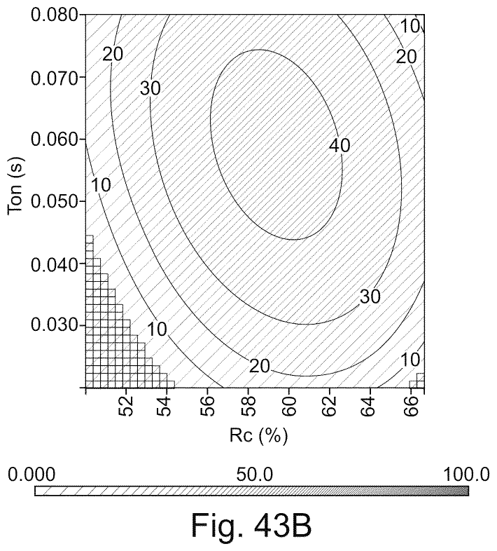

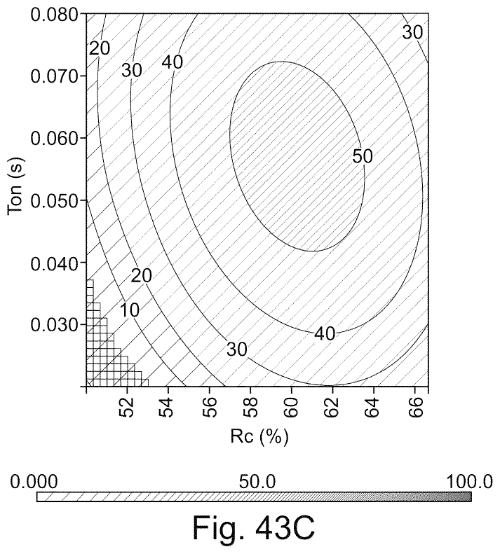

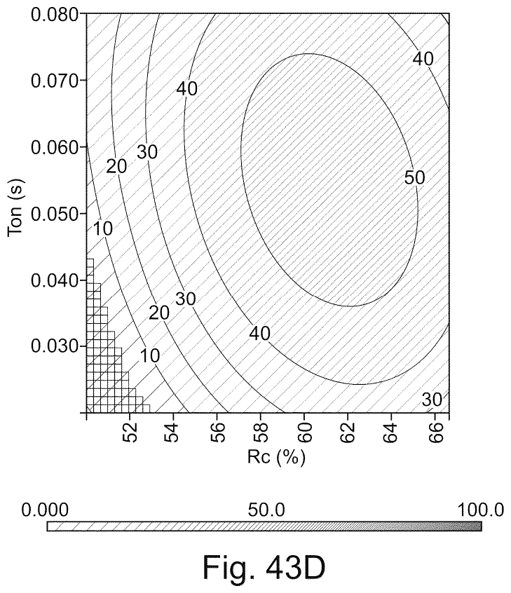

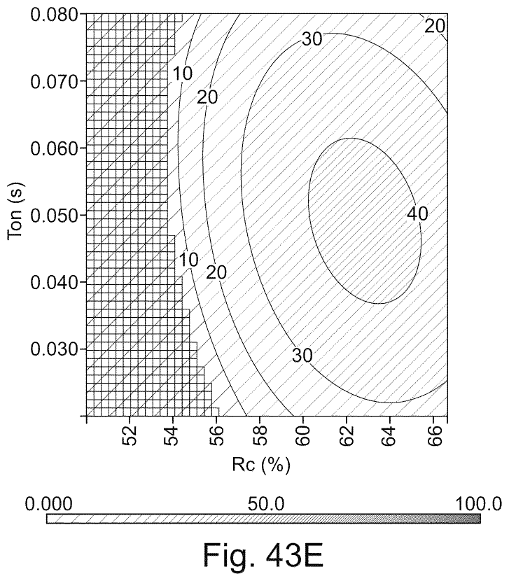

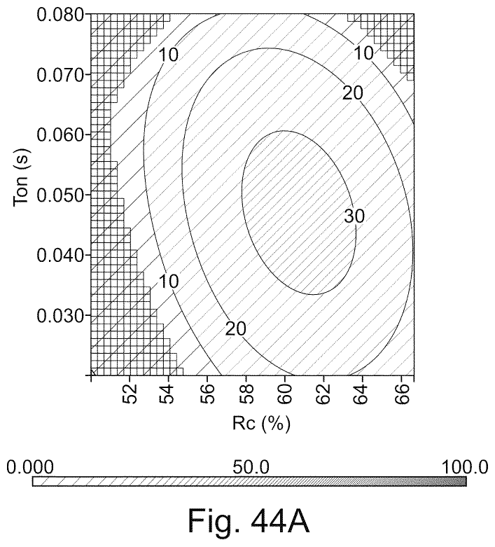

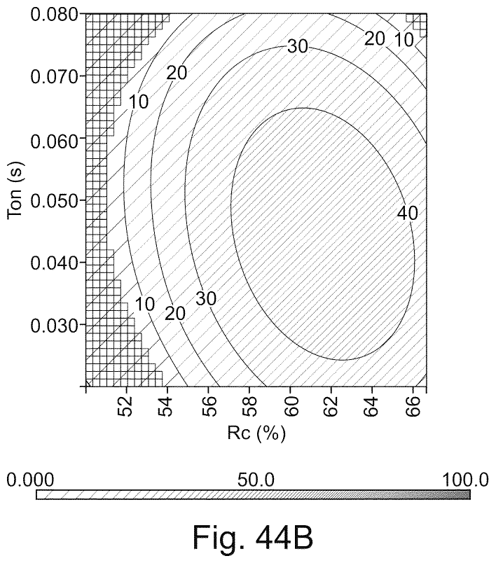

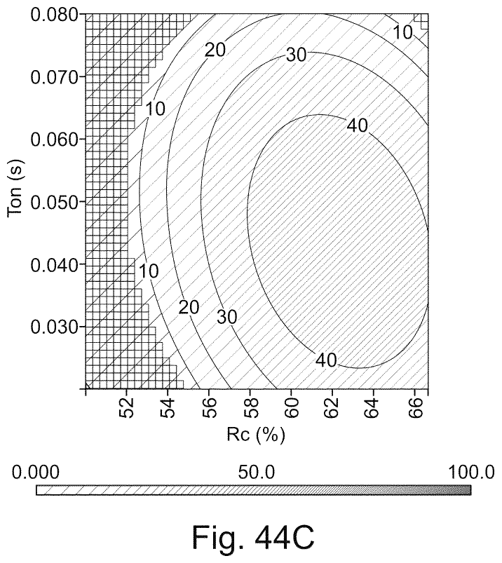

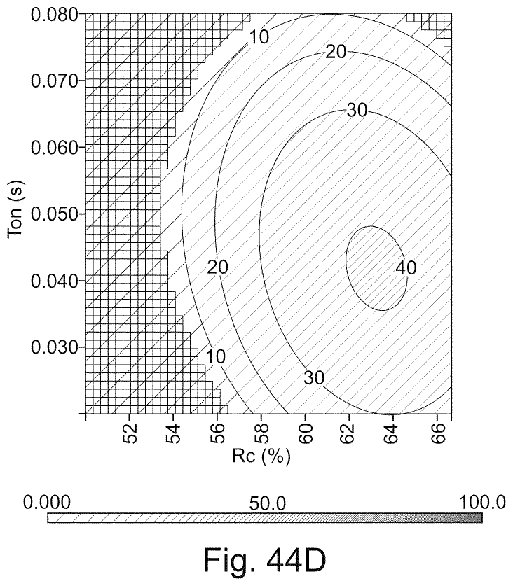

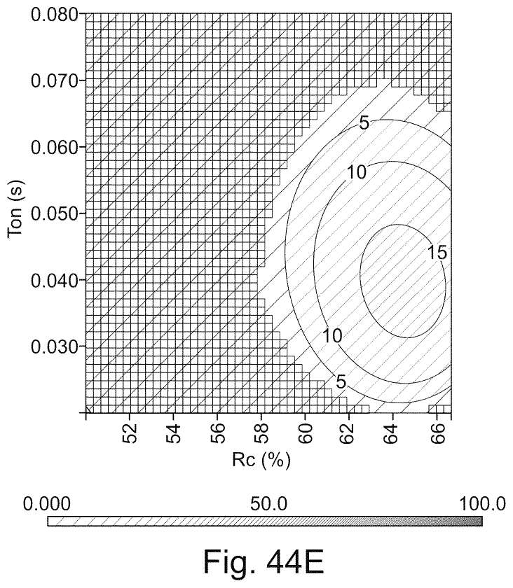

This thus gives response surfaces, as illustrated in FIGS. 40 to 45, illustrating ranges of values of various parameters for which satisfactory make-up removal effectiveness is found.

The make-up removal effectiveness is considered to be satisfactory in accordance with the same criteria as stated above, that is to say for a delta E greater than or equal to 15 and a make-up removal percentage greater than 65%.

In the optimization plan, three parameters are fixed, namely:
the total concentration of surfactant(s) (%) in the composition,
the peak acoustic intensity I (in W/cm²), and
the duration for which the surface to be cleaned is subjected to the acoustic waves.

Ranges of optimum values are determined for the other two parameters, namely the pulse duration Tₒₙ and the duty cycle Tₒₙ/T_{off}.

The ranges of optimum values are defined as those meeting the abovementioned make-up removal criteria. On the illustrated response curves, these correspond to the areas located above the isobar 0.

These ranges of values are summarized in the table below, with reference to each of the figures illustrating the corresponding response surface.

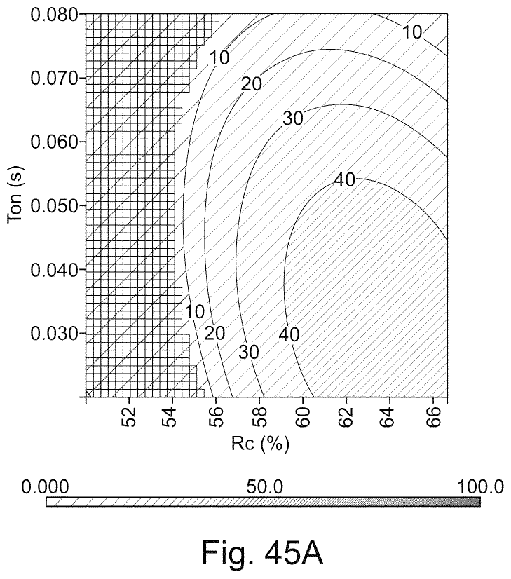

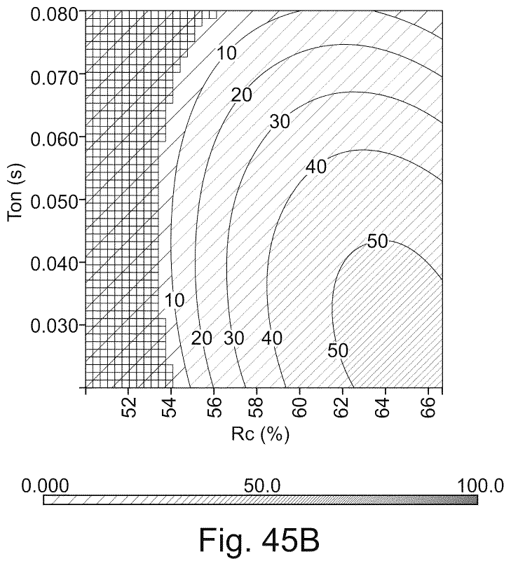

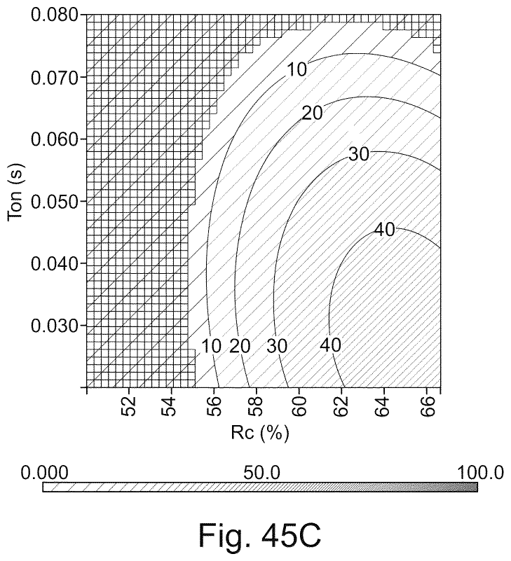

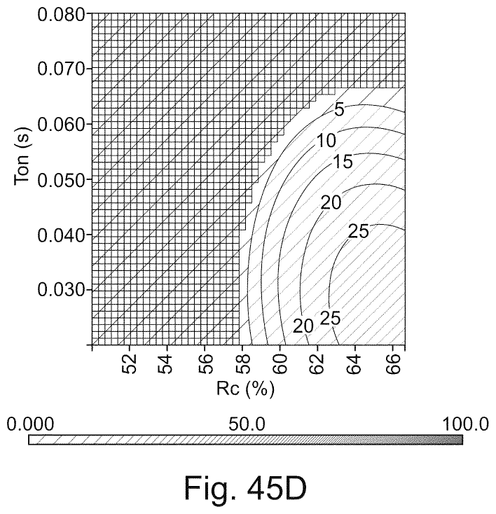

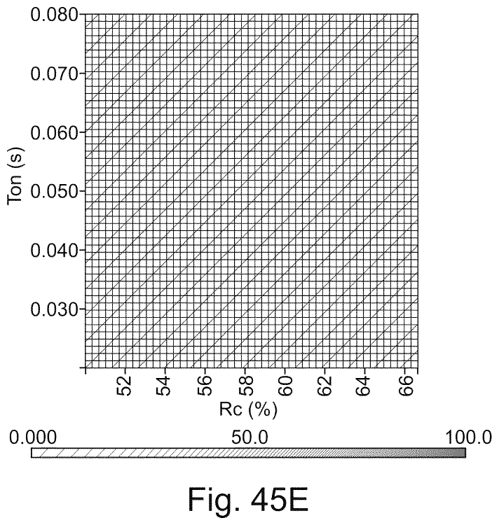

For some parameter values, as shown for example on the response surface of FIG. 45e, the response surface shows that there is no value of pulse duration and duty cycle Tₒₙ/T_{off} that meets the abovementioned make-up removal effectiveness criteria.

For other parameter values, as shown for example in FIGS. 40b to 40e, the entire domain under consideration for the optimization meets the make-up removal effectiveness criteria.

TABLE 2

| | I | Surfactant | | | Response |
|---|---|---|---|---|---|
| Duration (s) | (W/cm²) | (%) | Tₒₙ (s) | Duty cycle (%) | surface |
| 4 | 6.2 | 0.5 | [0.025; 0.080] | [50; 66.66] | FIG. 40a |
| | | | [0.020; 0.080] | [52; 65] | |
| | | [0.8; 1.5] | [0.020; 0.080] | [50; 66.66] | FIG. 40b |
| | | | [0.020; 0.080] | [50; 66.66] | FIG. 40c |
| | | | [0.020; 0.080] | [50; 66.66] | FIG. 40d |
| | | | [0.020; 0.080] | [50; 66.66] | FIG. 40e |
| 4 | 6.8 | 0.5 | [0.027; 0.080] | [50; 66.66] | FIG. 41a |
| | | | [0.020; 0.080] | [52; 66.66] | |
| | | 0.8 | [0.030; 0.080] | [50; 66.66] | FIG. 41b |
| | | | [0.020; 0.080] | [52; 66.66] | |
| | | 1.0 | [0.035; 0.080] | [50; 66.66] | FIG. 41c |
| | | | [0.020; 0.080] | [52.5; 66.66] | |
| | | 1.2 | [0.020; 0.080] | [54; 66.66] | FIG. 41d |
| | | | [0.035; 0.080] | [51.5; 66.66] | |
| | | 1.5 | [0.020; 0.080] | [57.5; 66.66] | FIG. 41e |
| | | | [0.038; 0.080] | [54; 66.66] | |
| 4 | 7.2 | 0.5 | [0.020; 0.080] | [50; 66.66] | FIG. 42a |
| | | 0.8 | [0.020; 0.080] | [50; 66.66] | FIG. 42b |
| | | | [0.030; 0.070] | [52; 66.66] | |
| | | 1.0 | [0.020; 0.080] | [50; 66.66] | FIG. 42c |
| | | | [0.030; 0.070] | [52.5; 66.66] | |
| | | 1.2 | [0.020; 0.080] | [54; 66.66] | FIG. 42d |
| | | | [0.025; 0.070] | [51.5; 66.66] | |
| | | 1.5 | [0.028; 0.045] | [57.5; 66.66] | FIG. 42e |
| 3 | 6.2 | 0.5 | [0.070; 0.080] | [54; 60.5] | FIG. 43a |
| | | | [0.055; 0.0775] | [56; 62] | |
| | | 0.8 | [0.020; 0.080] | [56; 66.66] | FIG. 43b |
| | | | [0.040; 0.080] | [51; 66.66] | |
| | | 1.0 | [0.020; 0.080] | [54; 66.66] | FIG. 43c |
| | | | [0.035; 0.080] | [51.5; 66.66] | |
| | | 1.2 | [0.020; 0.080] | [54; 66.66] | FIG. 43d |
| | | | [0.040; 0.080] | [51.5; 66.66] | |
| | | 1.5 | [0.020; 0.080] | [57; 66.66] | FIG. 43e |
| | | | [0.035; 0.080] | [55; 66.66] | |
| 3 | 6.8 | 0.5 | [0.020; 0.065] | [56; 66.66] | FIG. 44a |
| | | | [0.035; 0.075] | [53.5; 64] | |
| | | 0.8 | [0.020; 0.080] | [55; 64.5] | FIG. 44b |
| | | | [0.030; 0.072] | [53; 66.66] | |
| | | 1.0 | [0.032; 0.070] | [54; 66.66] | FIG. 44c |
| | | | [0.020; 0.080] | [56; 64.5] | |
| | | 1.2 | [0.032; 0.070] | [55.5; 66.66] | FIG. 44d |
| | | | [0.020; 0.080] | [58; 64] | |
| | | 1.5 | [0.025; 0.063] | [61.5; 66.66] | FIG. 44e |
| | | | [0.030; 0.058] | [60; 66.66] | |
| 3 | 7.2 | 0.5 | [0.020; 0.080] | [57; 65] | FIG. 45a |
| | | | [0.030; 0.065] | [55; 66.66] | |
| | | 0.8 | [0.020; 0.080] | [58; 66.66] | FIG. 45b |
| | | | [0.020; 0.068] | [55; 66.66] | |
| | | 1.0 | [0.020; 0.0725] | [58.5; 66.66] | FIG. 45c |
| | | | [0.020; 0.0575] | [56; 66.66] | |
| | | 1.2 | [0.020; 0.060] | [61; 66.66] | FIG. 45d |
| | | | [0.020; 0.046] | [58.5; 66.66] | |
| | | 1.5 | — | — | FIG. 45e |
| | | | — | — | |

1. A method for cleansing human keratin materials,
comprising the step consisting in subjecting a surface of said keratin materials to a cosmetic composition containing at least one surfactant, and to acoustic waves emitted by at least one ultrasonic transducer excited by a pulsed or non-pulsed electrical signal, having at least one frequency component of between 20 kHz and 100 kHz, with a duty cycle Ton/Toff, when the signal is pulsed, of between 20 and 100%, with an acoustic intensity Isata on said surface of at least 0.1 W/cm² and with a pulse duration Ton, when the signal is pulsed, of between 0.01 and 1 s, so as to generate, within the cosmetic composition, bubbles in the vicinity of the surface of said materials to be cleansed and to cause them to burst.

2. The method according to claim 1, the acoustic intensity Isata being between 0.1 and 10 W/cm².

3. according to claim 1, said frequency being between 30 and 100 kHz.

4. according to claim 1, the electrical signal being pulsed, said pulse duration Ton being between 0.01 s and 0.1 s.

5. according to claim 1, the electrical signal being pulsed, said duty cycle being between 20% and 100%.

6. according to claim 1, the composition having a total concentration of surfactant(s) of between 0.01% and 20% by weight with respect to the total mass of the composition.

7. The method according to claim 1, the distance between the transducer and said surface being between 1 and 30 mm.

8. The method according to claim 1, said surface being subjected to the acoustic waves for a duration of between 0.2 and 10 s.

9. The method according to claim 1, wherein an activation voltage is delivered to the transducer making it possible to obtain a peak acoustic intensity I which is greater than or equal to 5 W/cm2.

10. The method according claim 1, with I between 5 and 10 W/cm2.

11. The method according to claim 1, said surface being subjected to the acoustic waves for a duration which is equal to 4 s+/−0.4 s, the peak acoustic intensity being equal to 6.2 W/cm²+/−0.6 W/cm², and:
the composition having a total concentration of surfactant(s) of between 0.8 and 1.5%, the pulse duration Ton being between 0.020 and 0.080 s and the duty cycle being between 50 and 67%, or
the composition having a total concentration of surfactant(s) which is equal to 0.5%+/−0.1%, and
the pulse duration Ton being between 0.025 and 0.080 s and the duty cycle being between 50 and 67%, or
the pulse duration Ton being between 0.020 and 0.080 s and the duty cycle being between 52 and 65%.

12. The method according to claim 1, said surface being subjected to the acoustic waves for a duration which is equal to 4 s+/−0.4 s, the peak acoustic intensity being equal to 6.8 W/cm²+/−0.7 W/cm², and:
the composition having a total concentration of surfactant(s) which is equal to 0.5%+/−0.1%, and
the pulse duration Ton being between 0.027 and 0.080 s and the duty cycle being between 50 and 67%, or
the pulse duration Ton being between 0.020 and 0.080 s and the duty cycle being between 52 and 67%,
or
the composition having a total concentration of surfactant(s) which is equal to 0.8%+/−0.1%, and
the pulse duration Ton being between 0.030 and 0.080 s and the duty cycle being between 50 and 67%, or
the pulse duration Ton being between 0.020 and 0.080 s and the duty cycle being between 52 and 67%,
or
the composition having a total concentration of surfactant(s) which is equal to 1.0%+/−0.1%, and
the pulse duration Ton being between 0.035 and 0.080 s and the duty cycle being between 50 and 67%, or
the pulse duration Ton being between 0.020 and 0.080 s and the duty cycle being between 52.5 and 67%,
or
the composition having a total concentration of surfactant(s) which is equal to 1.2%+/−0.1%, and
the pulse duration Ton being between 0.035 and 0.080 s and the duty cycle being between 51.5 and 67%, or
the pulse duration Ton being between 0.020 and 0.080 s and the duty cycle being between 54 and 67%,
or
the composition having a total concentration of surfactant(s) which is equal to 1.5%+/−0.1%, and
the pulse duration Ton being between 0.038 and 0.080 s and the duty cycle being between 54 and 67%, or
the pulse duration Ton being between 0.020 and 0.080 s and the duty cycle being between 57.5 and 67%.

13. The method according to claim 1, said surface being subjected to the acoustic waves for a duration which is equal to 4 s+/−0.4 s, the peak acoustic intensity being equal to 7.2 W/cm²+/−0.7 W/cm², and:
the composition having a total concentration of surfactant(s) which is equal to 0.5%+/−0.1%, the pulse duration Ton being between 0.020 and 0.080 s and the duty cycle being between 50 and 67%,
or
the composition having a total concentration of surfactant(s) which is equal to 0.8%+/−0.1%, and
the pulse duration Ton being between 0.030 and 0.070 s and the duty cycle being between 51 and 67%, or
the pulse duration Ton being between 0.020 and 0.080 s and the duty cycle being between 52.5 and 67%,
or
the composition having a total concentration of surfactant(s) which is equal to 1.0%+/−0.1%, and
the pulse duration Ton being between 0.030 and 0.070 s and the duty cycle being between 52.5 and 67%, or
the pulse duration Ton being between 0.020 and 0.080 s and the duty cycle being between 55 and 67%,
or
the composition having a total concentration of surfactant(s) which is equal to 1.2%+/−0.1%, and
the pulse duration Ton being between 0.025 and 0.070 s and the duty cycle being between 56 and 67%, or
the pulse duration Ton being between 0.020 and 0.080 s and the duty cycle being between 59 and 67%,
or
the composition having a total concentration of surfactant(s) which is equal to 1.5%+/−0.1%, the pulse duration Ton being between 0.028 and 0.045 s and the duty cycle being between 59 and 63%.

14. The method according to claim 1, said surface being subjected to the acoustic waves for a duration which is equal to 3 s+/−0.3 s, the peak acoustic intensity being equal to 6.2 W/cm²+/−0.6 W/cm², and:
the composition having a total concentration of surfactant(s) which is equal to 0.5%+/−0.1%, and
the pulse duration Ton being between 0.070 and 0.080 s and the duty cycle being between 54 and 60.5%, or
the pulse duration Ton being between 0.055 and 0.078 s and the duty cycle being between 56 and 62%,
or
the composition having a total concentration of surfactant(s) which is equal to 0.8%+/−0.1%, and
the pulse duration Ton being between 0.040 and 0.080 s and the duty cycle being between 51 and 67%, or
the pulse duration Ton being between 0.020 and 0.080 s and the duty cycle being between 56 and 67%,
or
the composition having a total concentration of surfactant(s) which is equal to 1.0%+/−0.1%, and
the pulse duration Ton being between 0.035 and 0.080 s and the duty cycle being between 51.5 and 67%, or
the pulse duration Ton being between 0.020 and 0.080 s and the duty cycle being between 54 and 67%,
or
the composition having a total concentration of surfactant(s) which is equal to 1.2%+/−0.1%, and
the pulse duration Ton being between 0.040 and 0.080 s and the duty cycle being between 51.5 and 67%, or
the pulse duration Ton being between 0.020 and 0.080 s and the duty cycle being between 54 and 67%,
or
the composition having a total concentration of surfactant(s) which is equal to 1.5%+/−0.1%, and
the pulse duration Ton being between 0.035 and 0.080 s and the duty cycle being between 55 and 67%, or
the pulse duration Ton being between 0.020 and 0.080 s and the duty cycle being between 57 and 67%.

15. The method according to claim 1, said surface being subjected to the acoustic waves for a duration which is equal to 3 s+/−0.3 s, the peak acoustic intensity being equal to 6.8 W/cm²+/−0.7 W/cm², and:
the composition having a total concentration of surfactant(s) which is equal to 0.5%+/−0.1%, and
the pulse duration Ton being between 0.020 and 0.065 s and the duty cycle being between 56 and 67%, or
the pulse duration Ton being between 0.035 and 0.075 s and the duty cycle being between 53.5 and 64%,
or
the composition having a total concentration of surfactant(s) which is equal to 0.8%+/−0.1%, and
the pulse duration Ton being between 0.030 and 0.072 s and the duty cycle being between 53 and 67%, or
the pulse duration Ton being between 0.020 and 0.080 s and the duty cycle being between 55 and 64.5%,
or
the composition having a total concentration of surfactant(s) which is equal to 1.0%+/−0.1%, and
the pulse duration Ton being between 0.032 and 0.070 s and the duty cycle being between 54 and 67%, or
the pulse duration Ton being between 0.020 and 0.080 s and the duty cycle being between 56 and 64.5%,
or
the composition having a total concentration of surfactant(s) which is equal to 1.2%+/−0.1%, and
the pulse duration Ton being between 0.032 and 0.070 s and the duty cycle being between 55.5 and 67%, or
the pulse duration Ton being between 0.020 and 0.080 s and the duty cycle being between 58 and 64%,
or
the composition having a total concentration of surfactant(s) which is equal to 1.5%+/−0.1%, and
the pulse duration Ton being between 0.025 and 0.063 s and the duty cycle being between 61.5 and 67%, or
the pulse duration Ton being between 0.030 and 0.058 s and the duty cycle being between 60 and 67%.

16. The method according to claim 1, said surface being subjected to the acoustic waves for a duration which is equal to 3 s+/−0.3 s, the peak acoustic intensity being equal to 7.2 W/cm²+/−0.7 W/cm², and:
the composition having a total concentration of surfactant(s) which is equal to 0.5%+/−0.1%, and
the pulse duration Ton being between 0.020 and 0.080 s and the duty cycle being between 57 and 65%, or
the pulse duration Ton being between 0.030 and 0.065 s and the duty cycle being between 55 and 67%,
or
the composition having a total concentration of surfactant(s) which is equal to 0.8%+/−0.1%, and
the pulse duration Ton being between 0.020 and 0.080 s and the duty cycle being between 58 and 67%, or
the pulse duration Ton being between 0.020 and 0.068 s and the duty cycle being between 55 and 67%,
or
the composition having a total concentration of surfactant(s) which is equal to 1.0%+/−0.1%, and
the pulse duration Ton being between 0.020 and 0.0725 s and the duty cycle being between 58.5 and 67%, or
the pulse duration Ton being between 0.020 and 0.0575 s and the duty cycle being between 56 and 67%,
or
the composition having a total concentration of surfactant(s) which is equal to 1.2%+/−0.1%, and
the pulse duration Ton being between 0.020 and 0.060 s and the duty cycle being between 61 and 67%, or
the pulse duration Ton being between 0.020 and 0.046 s and the duty cycle being between 58.5 and 67%.

17. A device for treating human keratin materials the device comprising at least one ultrasonic transducer designed to emit acoustic waves into a fluid present in the vicinity of said materials to be cleansed, the transducer being supplied with power by a current generator delivering a pulsed or non-pulsed electrical signal, this electrical signal having:
at least one frequency component of between 20 kHz and 100 kHz,
a duty cycle Ton/Toff, when the signal is pulsed, of between 20 and 100%, and/or,
a pulse duration Ton, when the signal is pulsed, of between 0.01 and 1 s,
the acoustic intensity Isata on the surface of said keratin materials being at least 0.1 W/cm².
